(19) United States
(12) Patent Application Publication
(10) Pub. No.: US 2011/0212080 A1
(43) Pub. Date: Sep. 1, 2011

(54) UREA DERIVATIVES AS ANTIBACTERIAL AGENTS

(76) Inventors: Umar Faruk Mansoor, Farmingham, MA (US); Panduranga Adulla Reddy, Walpole, MA (US); M. Arshad Siddiqui, Newton, MA (US)

(21) Appl. No.: 13/057,505

(22) PCT Filed: Jul. 28, 2009
(86) PCT No.: PCT/US2009/051898

§ 371 Date: May 17, 2011

Related U.S. Application Data
(60) Provisional application No. 61/085,982, filed on Aug. 4, 2008.

(51) Int. Cl. A61K 39/395; C07D 241/04; A61K 31/495; C07D 403/10; A61K 31/496; C07D 413/10; A61K 31/5377; C07D 211/10; A61K 31/451; C07D 401/14; C07D 413/14; A61K 31/497; A61P 31/00

(52) U.S. Cl. 424/130.1; 544/390; 514/255.01; 544/357; 514/252.11; 544/121; 514/235.8; 546/245; 514/315; 544/371; 514/254.05; 544/360; 514/253.01

(57) ABSTRACT

This invention relates to compounds of the Formula (I): or a pharmaceutically acceptable salt, solvate, ester or isomer thereof, which is useful for the treatment of diseases or conditions mediated by LpxC.

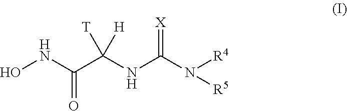

### FIELD OF THE INVENTION

This invention rates generally to heterocycles that can inhibit UDP-3-O-(R-3-hydroxymyristoyl)-N-acetylglucosamine deacetylase (LpxC), and as a result have antimicrobial activity.

### BACKGROUND OF THE INVENTION

Lipid A is the hydrophobic anchor of lipopolysaccharide (LPS) and faints the major lipid component of the outer monolayer of the outer membrane of gram-negative bacteria. Lipid A is required for bacterial growth and inhibition of its biosynthesis is lethal to the bacteria. Furthermore, blocking Lipid A biosynthesis increases the sensitivity of bacteria to other antibiotics.

One of the key enzymes of bacterial lipid A biosynthesis is LpxC. LpxC catalyzes the removal of the N-acetyl group of UDP-3-O-(R-3-hydroxymyristoyl)-N-acetylglucosamine. The LpxC enzyme is essential in gram negative bacteria for the biosynthesis of Lipid A, and it is notably absent from mammalian genomes. Since LpxC is essential for Lipid A biosynthesis and inhibition of Lipid A biosynthesis is lethal to bacteria, inhibitors of LpxC have utility as antibiotics. In addition, the absence of LpxC from mammalian genomes reduces potential toxicity of LpxC inhibitors in mammals. Accordingly, LpxC is an attractive target for antibacterial drug discovery.

U.S. Pat. No. 5,925,659 teaches that certain heterocyclic hydroxamate compounds, in particular oxazoline compounds, have the ability to inhibit LpxC.

WO2004/00744 refers to N-Hydroxyamide derivatives having LpxC inhibitory activity and thus possessing antibacterial activity.

WO2004/062601 also refers to small molecule inhibitors of LpxC.

WO2007/064732 refers to N-Hydroxyamide derivatives having LpxC inhibitor activity and thus possessing antibacterial activity.

WO2008/027466 also refers to small molecule inhibitors of LpxC.

WO2001/144178 urea derivatives having metalloenzyme (peptide deformylase) inhibitory activity and thus possessing antimicrobial and antibiotic activity.

There is a need in the art for small molecule inhibitors of LpxC as potential antibacterial agents.

### SUMMARY OF THE INVENTION

In its many embodiments, the present invention provides a novel class of compounds as inhibitors of LpxC, methods of preparing such compounds, pharmaceutical compositions comprising one or more such compounds, methods of preparing pharmaceutical formulations comprising one or more such compounds, and methods of treatment, prevention, inhibition or amelioration of one or more diseases associated with LpxC, using such compounds or pharmaceutical compositions.

In one embodiment, the present application discloses a compound, or pharmaceutically acceptable salt, solvate, or ester of said compound, said compound having the general structure shown in formula (I):

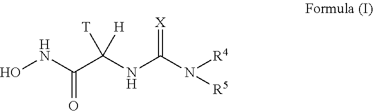

, wherein:

(i) T is selected from the group consisting of H, alkyl, alkenyl and alkynyl, wherein said alkyl, alkenyl and alkynyl can be unsubstituted or optionally independently substituted with one or more moieties selected from the group consisting of aryl, aralkyl, heteroaryl, heteroaralkyl, cycloalkyl, cycloalkylalkyl, cycloalkenyl, cycloalkenylalkyl, heterocyclyl, heterocyclenyl, heterocycloalkylalkyl, heterocyclenylalkyl, —OH, alkoxyl, —O-alkenyl, —O-alkynyl, hydroxyalkyl, hydroxyalkenyl, —O-aryl, —O-aralkyl, —SH, —S-alkyl, —S-alkenyl, —S-alkynyl, —S-aryl, —S-aralkyl, —NR¹R², -alkyl-NR¹R² and -alkenyl-NR¹R²,

wherein R¹ and R² are independently selected from the group consisting H, alkyl, alkenyl, alkynyl, aryl, cycloalkyl, cycloalkenyl, aralkyl, cycloalkylalkyl, cycloalkenylalkyl, heteroaryl, heteroaralkyl, or

R¹ and R² together with the N atom to which each is attached form heterocyclyl, heterocyclenyl, or heteroaryl; or

T and H together with the C atom to each is attached form spirocycloalkyl or spiroheterocyclyl, wherein each of said spirocycloalkyl and spiroheterocyclyl can be unsubstituted or optionally independently substituted with one or more moieties selected from the group consisting of alkyl, alkenyl, alkenyl, aryl, cycloalkenyl, cycloalkyl, aralkyl, aralkenyl, cycloalkenylalkyl, cycloalkylalkyl, halo and haloalkyl;

(ii) X is O, S or NH;

(iii) R⁴ and R⁵ are independently selected from the group consisting of hydrogen or (C₁-C₆)alkyl, wherein said (C₁-C₆)alkyl is substituted with aryl wherein said aryl can be unsubstituted or optionally independently substituted with one or more moieties selected from the group consisting of alkynyl, halo, aryl, heteroaryl, heterocyclenyl or heterocyclyl, wherein said alkynyl, heteroaryl, heterocyclenyl or heterocyclyl can be unsubstituted or substituted with an additional aryl; or

R⁴ and R⁵ together with the N atom to which each is attached, form a heterocyclyl or heterocyclenyl, wherein each of said heterocyclyl and heterocyclenyl is substituted with A; or

R⁴ and R⁵ together with the N atom to which each is attached form a heterocyclic structure represented by the structure:

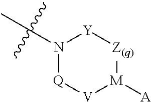

wherein Y, Q, Z, or V are each independently selected from the group consisting of C(O), C(S), C(NH), S(O), S(O)₂ and C(R⁶R⁷), wherein q is 0-1, wherein each of R⁶ and R⁷ are independently selected from the group consisting of H, alkyl, and alkenyl, wherein each of said alkyl or alkenyl can be unsubstituted or optionally independently substituted with one or more moieties selected from the group consisting of H, alkyl, alkenyl, alkynyl, aryl, cycloalkenyl, cycloalkyl, aralkyl, aralkenyl, cycloalkenylalkyl, cycloalkylalkyl, halo and haloalkyl;

further wherein M is N or CR, wherein R is H, halo, alkyl, alkenyl, alkynyl, aryl, aralkyl, cycloalkenyl, cycloalkyl, heteroaryl, heterocyclenyl, heterocyclyl, OH, —O-alkyl, —O-alkenyl, —O-alkynyl, —O-aryl, —O-aralkyl, —O-cycloalkenyl, —O-cycloalkyl, —O-heteroaryl, —O-heterocyclenyl, —O-heterocyclyl, —SH, —S-alkyl, —S-alkenyl, —S-alkynyl, —S-aralkyl, —S-cycloalkenyl, —S-cycloalkyl, —S-heteroaryl, —S-heterocyclenyl, or —S-heterocyclyl;

A is selected from the group consisting of, -aryl-alkynyl-aryl, -aryl-C(O)aralkyl, -aryl, -biaryl, alkynyl-aryl, -aryl-heteroaryl and -aryl-alkynyl-heteroaryl, wherein said, -aryl-alkynyl-aryl, —C(O)aralkyl, -aryl, -biaryl, -alkynyl-aryl, -aryl-heteroaryl and -aryl-alkynyl-heteroaryl can be unsubstituted or optionally independently substituted with one or more moieties selected from the group consisting of halo, haloalkyl, —N(R¹)(R²), haloalkoxyl, -alkyl-CN, hydroxyalkyl, —OH, heterocyclyl, heterocyclenyl, alkyl, alkenyl, dialkylaminoalkoxyl and heterocyclylalkoxyl.

The compounds of Formulae (I) are useful as inhibitors and may be useful the treatment and prevention of diseases associated with LpxC.

### DETAILED DESCRIPTION OF THE INVENTION

In its several embodiments, the present invention provides a novel class of inhibitors of LpxC, pharmaceutical compositions containing one or more of the compounds, methods of preparing pharmaceutical formulations comprising one or more such compounds, and methods of treatment, prevention or amelioration of microbial infections.

In one embodiment, the present invention provides compounds which are represented by structural Formulae (I) above or a pharmaceutically acceptable salt, solvate, ester or isomer thereof, wherein the various moieties are as described above.

In another embodiment, in formula (I), wherein T is hydroxyalkyl.

In another embodiment, in formula (I), wherein X is O.

In another embodiment, in formula (I), wherein each of said R⁴ and R⁵ is independently hydrogen or (C₁-C₆)alkyl, wherein said (C₁-C₆)alkyl is substituted with aryl, wherein said aryl can be unsubstituted or optionally independently substituted with alkynyl, halo or heteroaryl, wherein said alkynyl is substituted with an additional aryl.

In another embodiment, in formula (I), wherein said (C₁-C₆)alkyl can be straight chain alkyl or branched alkyl.

In another embodiment, in formula (I) wherein said alkyl is methyl, ethyl, or branched ethyl.

In another embodiment, in formula (I), wherein said alkynyl ethynyl.

In another embodiment, in formula (I), wherein said halo is bromo, chloro or fluro.

In another embodiment, in formula (I), wherein said heteroaryl is N-pyrazole.

In another embodiment, in formula (I), wherein said R⁴ and R⁵ together with the N atom to which each is attached form heterocyclyl, substituted with A.

In another embodiment, in formula (I), wherein said heterocyclyl is piperazinyl, piperidinyl or pyrollidinyl.

In another embodiment, in formula (I), wherein A is phenyl-ethynyl-phenyl, ethynyl-phenyl, phenyl-C(O)-benzyl, phenyl, biphenyl, phenyl-heteroaryl, phenyl-heteroaryl-heterocyclyl or phenyl-ethynyl-heteroaryl,

wherein said phenyl can be unsubstituted or optionally independently substituted with one or more moieties selected from the group consisting of chloro, bromo, —NH₂, dialkylamino, haloalkyl, haloalkoxyl and cyanoalkyl,

wherein said biphenyl can be unsubstituted or optionally independently substituted with one or more moieties selected from the group consisting of propyl, heterocyclyl, dimethylaminoethoxyl, and heterocyclylalkoxyl;

wherein said heteroaryl is selected from the group consisting of pyrimidinyl, pyridinyl, thiophenyl, thiazolyl, pyrazinyl and pyrazolyl, further wherein said heteroaryl can be unsubstituted or optionally independently substituted with one or more moieties selected from the group consisting of halo, alkyl, —NH₂ and heterocyclyl; and

wherein said heterocyclyl is selected from the group consisting of morpholinyl, piperazinyl, piperidinyl and pyrrolidinyl.

In another embodiment, in formula (I), wherein said heteroaryl is selected from the group consisting of 5-pyrimidinyl, 4-pyridinyl, 3-thiophenyl, 5-thiazolyl, 2-pyrazinyl and 5-pyrazolyl.

In another embodiment, in formula (I), wherein said heterocyclyl is selected from the group consisting of 4-morpholinyl, piperazinyl, piperidinyl and pyrrolidinyl.

In another embodiment, in formula (I), wherein said biphenyl is substituted with 4-morpholinylethoxyl.

In another embodiment, a compound of formula (II):

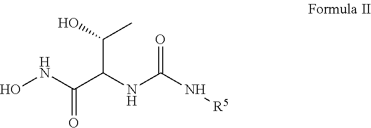

or pharmaceutically acceptable salt, solvate or ester thereof, wherein:
R⁵ is (C₁-C₂)alkyl, wherein said (C₂-C₂)alkyl is substituted with phenyl, herein said phenyl can be unsubstituted or optionally independently substituted with ethynyl, bromo or pyrazolyl, wherein said ethynyl is substituted with an additional aryl.

In another embodiment, a compound of formula (III):

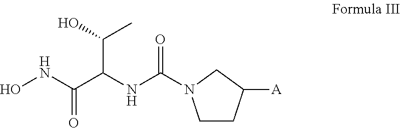

or pharmaceutically acceptable salt, solvate or ester thereof,
wherein A is phenyl-ethynyl-phenyl.

In another embodiment, a compound of formula (IVA):

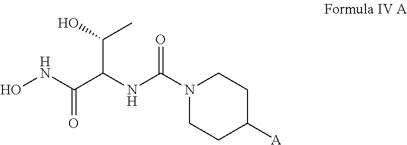

or pharmaceutically acceptable salt, solvate or ester thereof,
wherein A is phenyl, wherein said phenyl can be unsubstituted or optionally independently substituted with one or moieties selected from the group consisting of chloro, bromo, propyl, phenyl-ethynyl-phenyl, phenyl-C(O)benzyl, ethynyl-phenyl, biphenyl, wherein said biphenyl can be unsubstituted or substituted with propyl.

In another embodiment, a compound of formula (IVB):

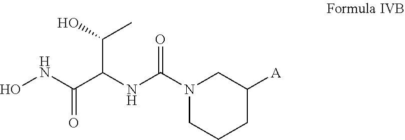

or pharmaceutically acceptable salt, solvate or ester thereof, A is phenyl substituted with chloro.

In another embodiment, a compound of formula (VA):

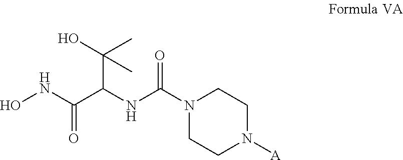

or pharmaceutically acceptable salt, solvate or ester thereof, wherein A is phenyl-ethynyl-phenyl or biphenyl, wherein said phenyl-ethynyl-phenyl or biphenyl can be unsubstituted car optionally independently substituted with one or more moieties selected from the group consisting of fluro, chloro, —NH₂, —N(Me)₂, —N(Et)₂, CF₃, —OCF₃, —CH₂—CN, —CH₂OH, morpholinylmethyl, —OH, piperazinyl, morpholinyl, dimethylaminoethoxyl, and morpholinylethoxyl.

In another embodiment, a compound of formula (VB):

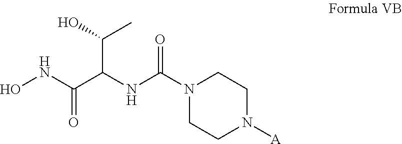

or pharmaceutically acceptable salt, solvate or ester thereof,
wherein A is phenyl-ethynyl or phenyl, wherein said phenyl of said phenyl-ethynyl or phenyl is substituted with heteroaryl, wherein said heteroaryl is selected from the group consisting of

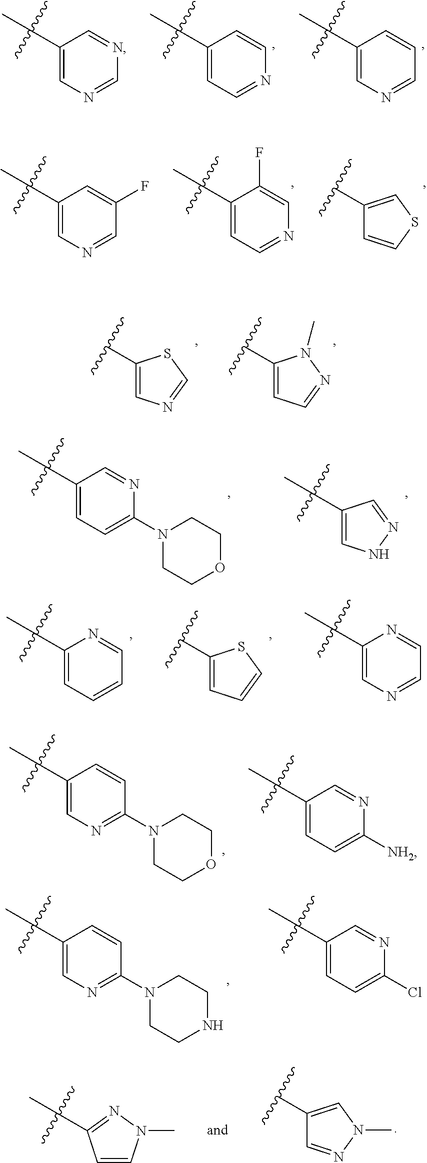

In another embodiment, a compound of formula (VI):

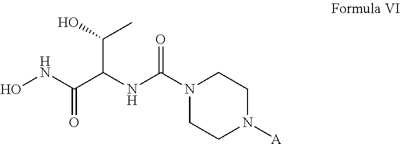

or pharmaceutically acceptable salt, solvate or ester thereof,
wherein A is biphenyl or phenyl-ethynyl-phenyl, wherein each of said biphenyl and phenyl-ethynyl-phenyl can be unsubstituted or substituted with piperazinyl.

In another embodiment, a compound of formula (VII):

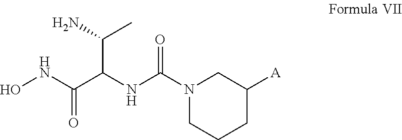

or phramaceutically acceptable salt, solvate or ester thereof,
wherein A is phenyl-ethynyl-phenyl or biphenyl, wherein each of said phenyl-ethynyl-phenyl and biphenyl can be unsubstituted or optionally independently substituted with morpholinyl or piperazinyl.

In another embodiment, a compound of formula (VIII):

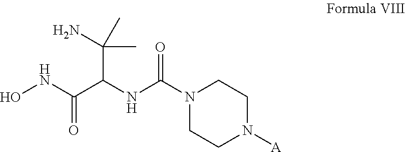

or pharmaceutically acceptable salt, solvate or ester thereof,
wherein A is biphenyl.

In another embodiment, a compound of formula (IX):

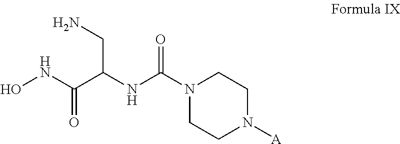

or pharmaceutically acceptable salt, solvate or ester thereof,
wherein A is phenyl-ethynyl-phenyl.

In another embodiment, the compounds of formula (I) selected from the group consisting of:

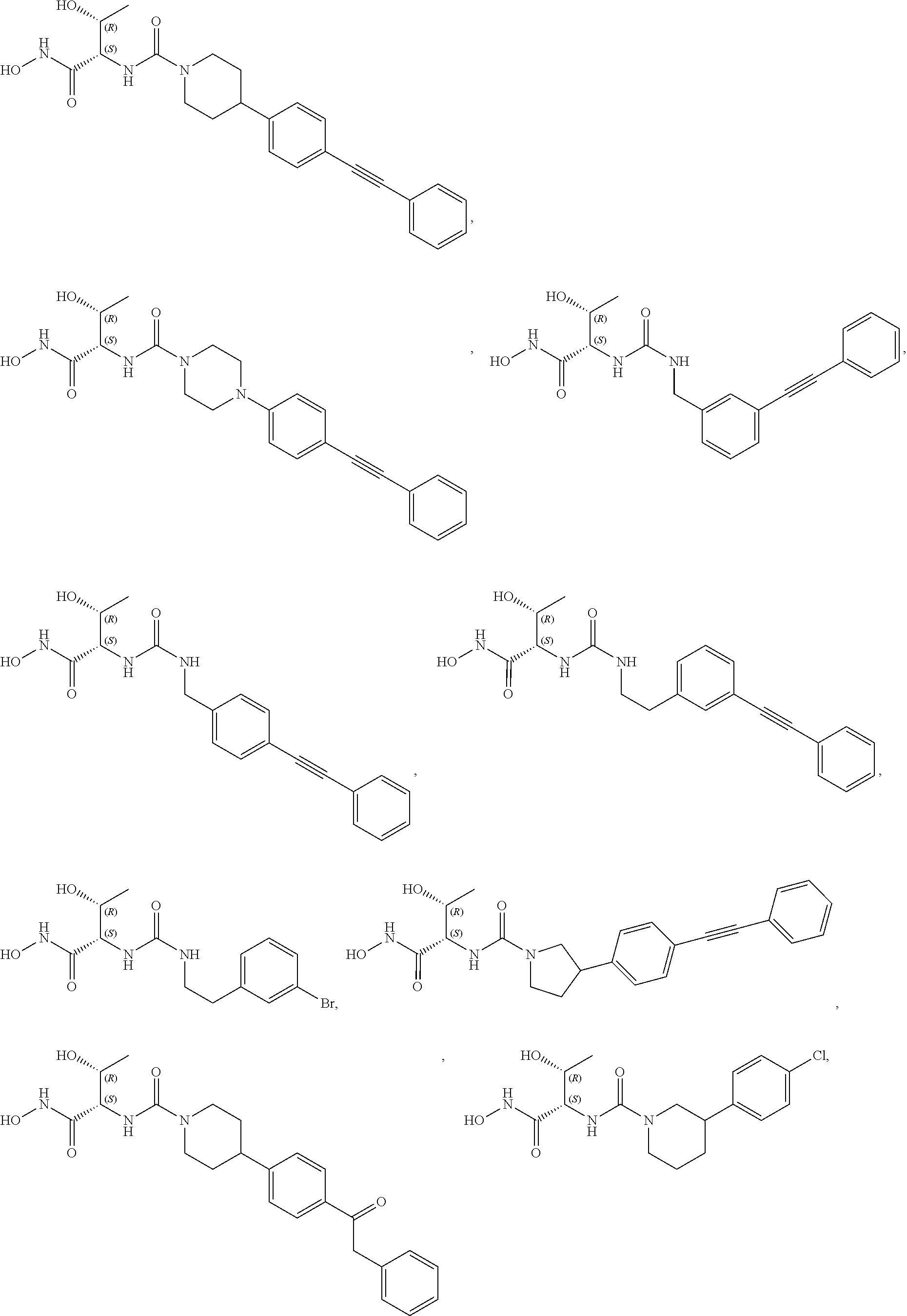

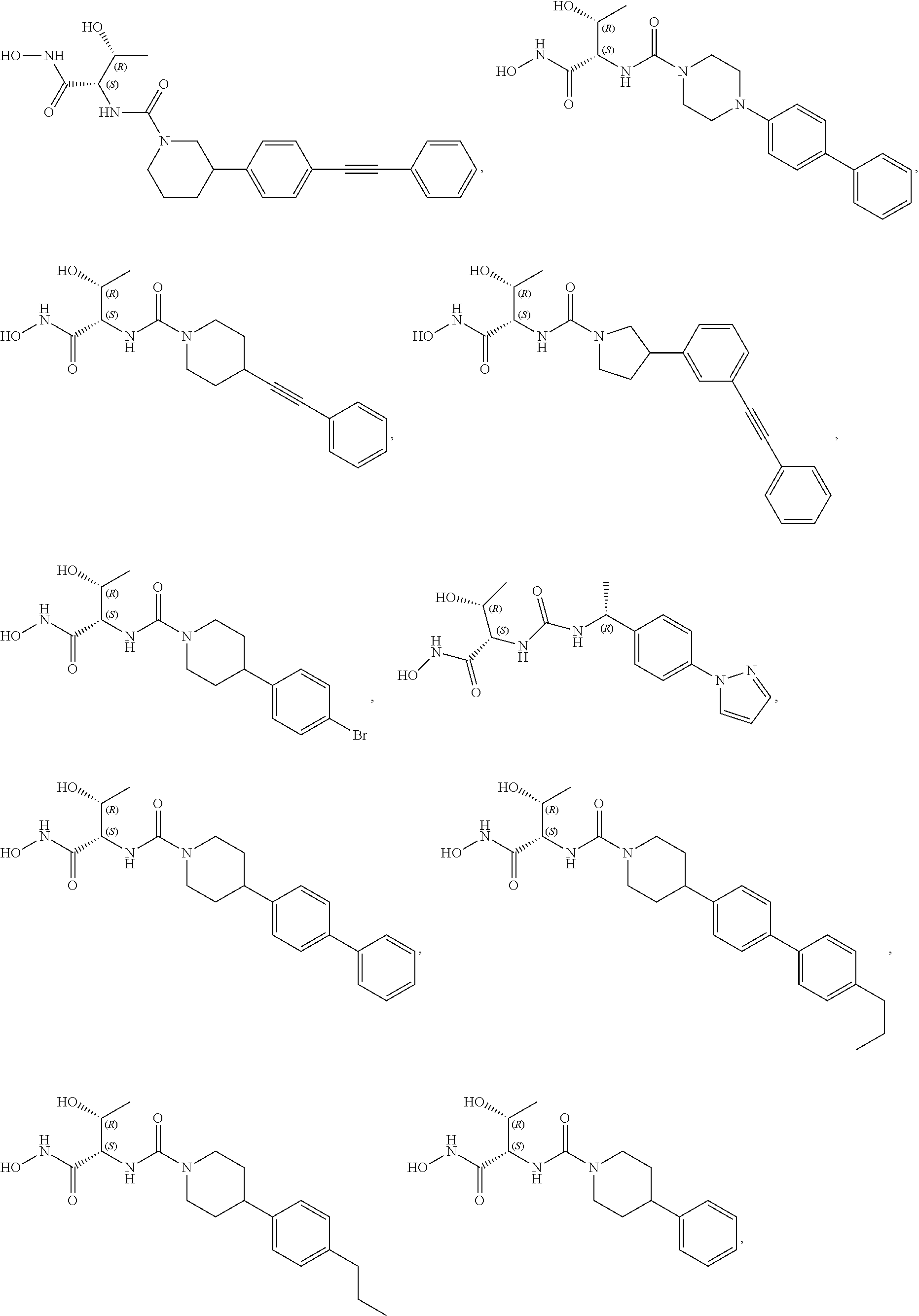

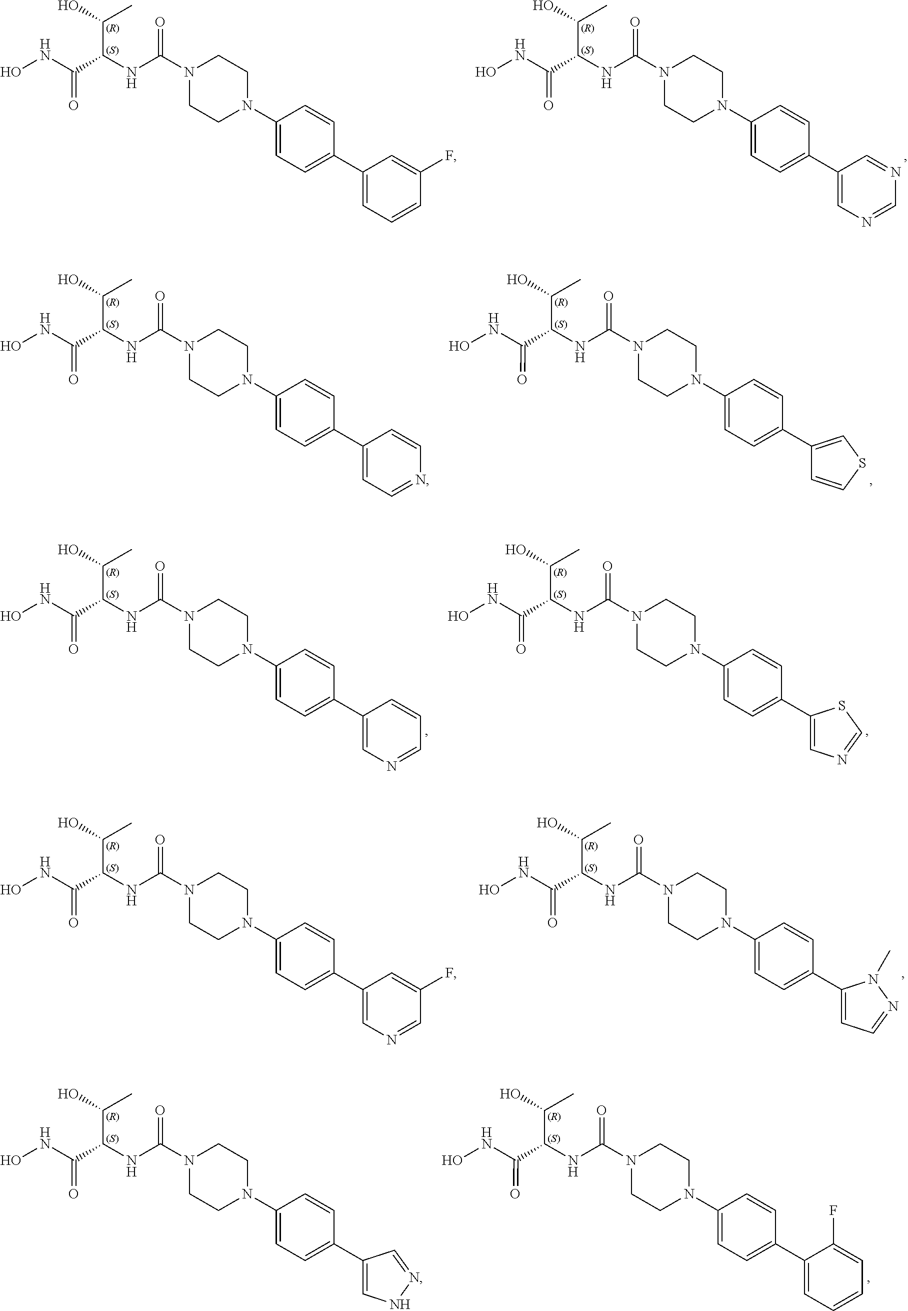

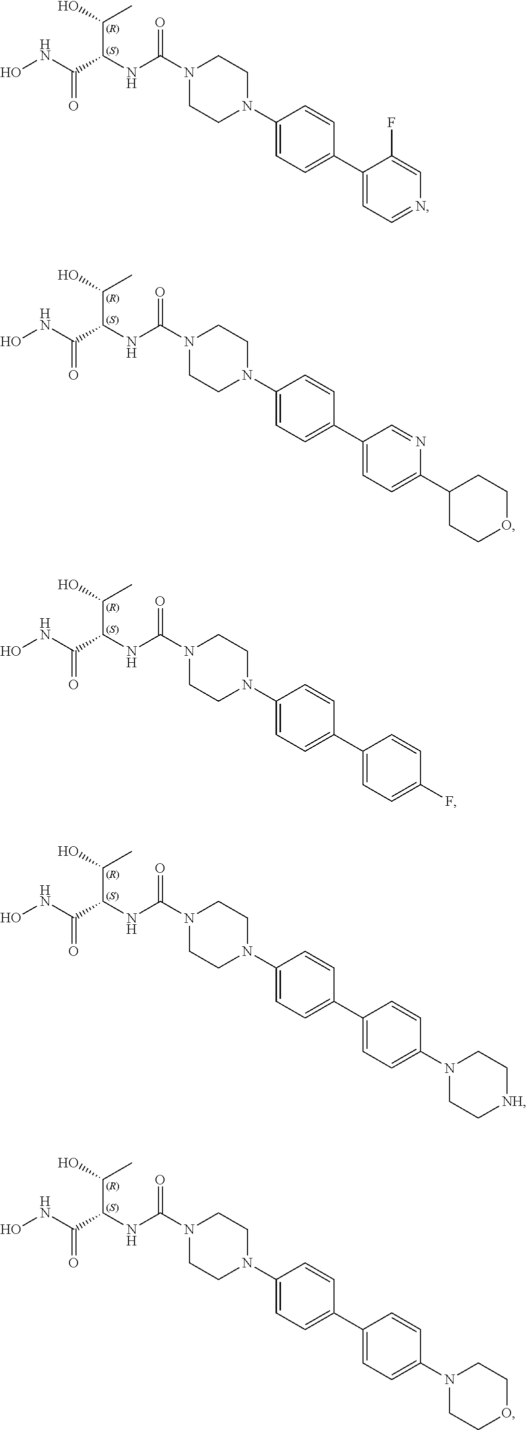

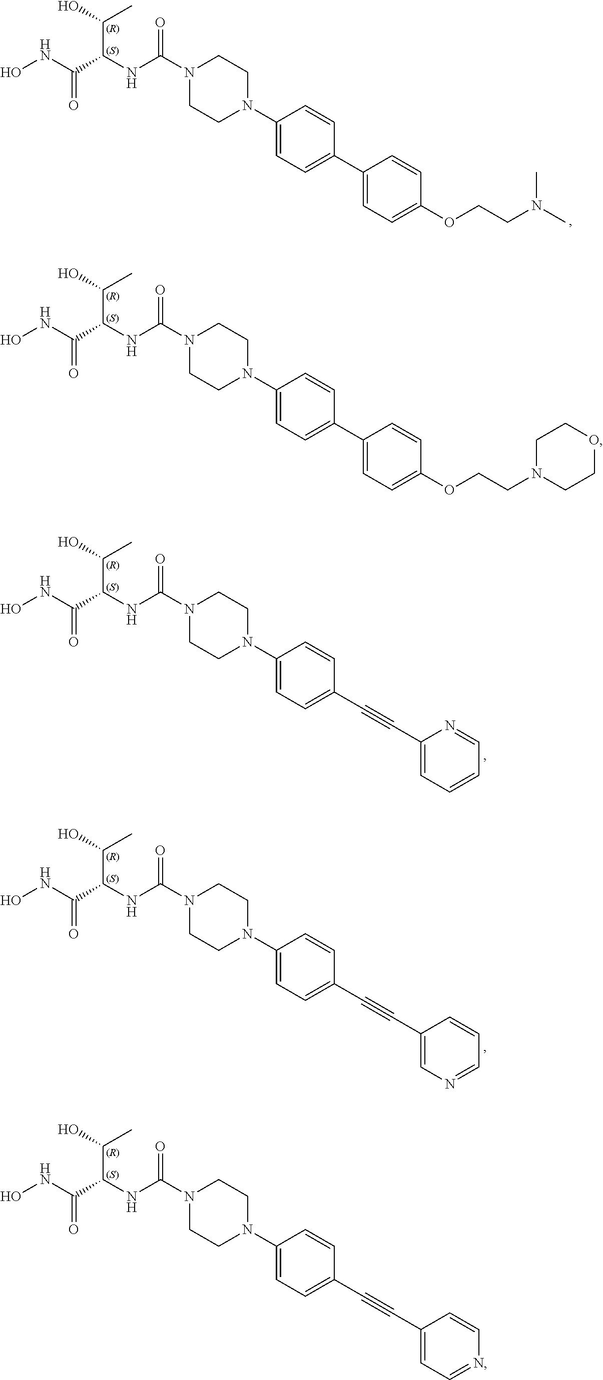

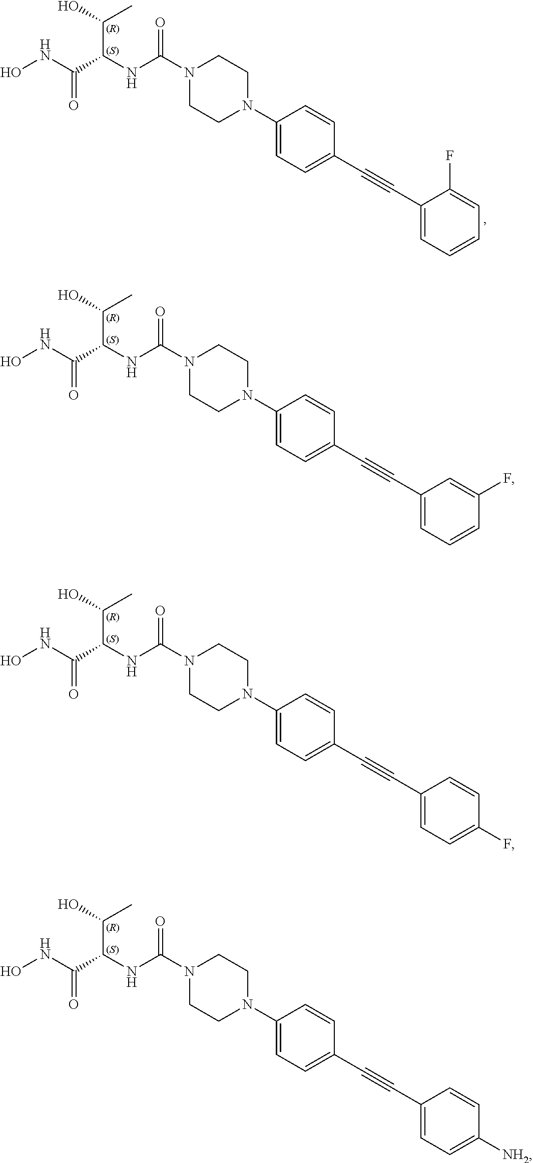

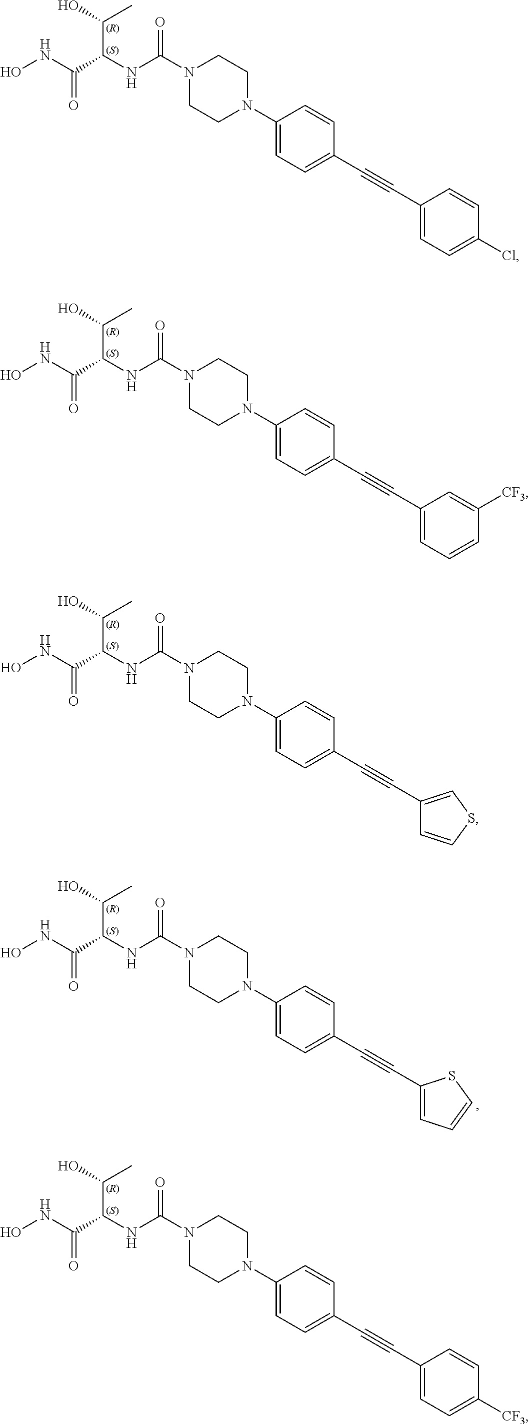

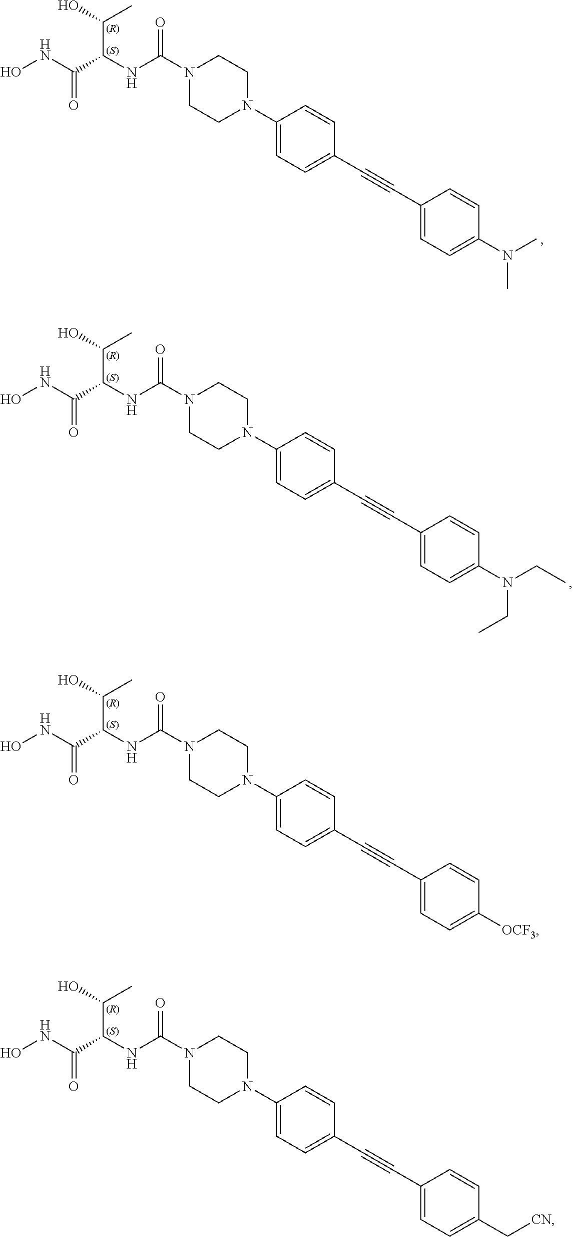

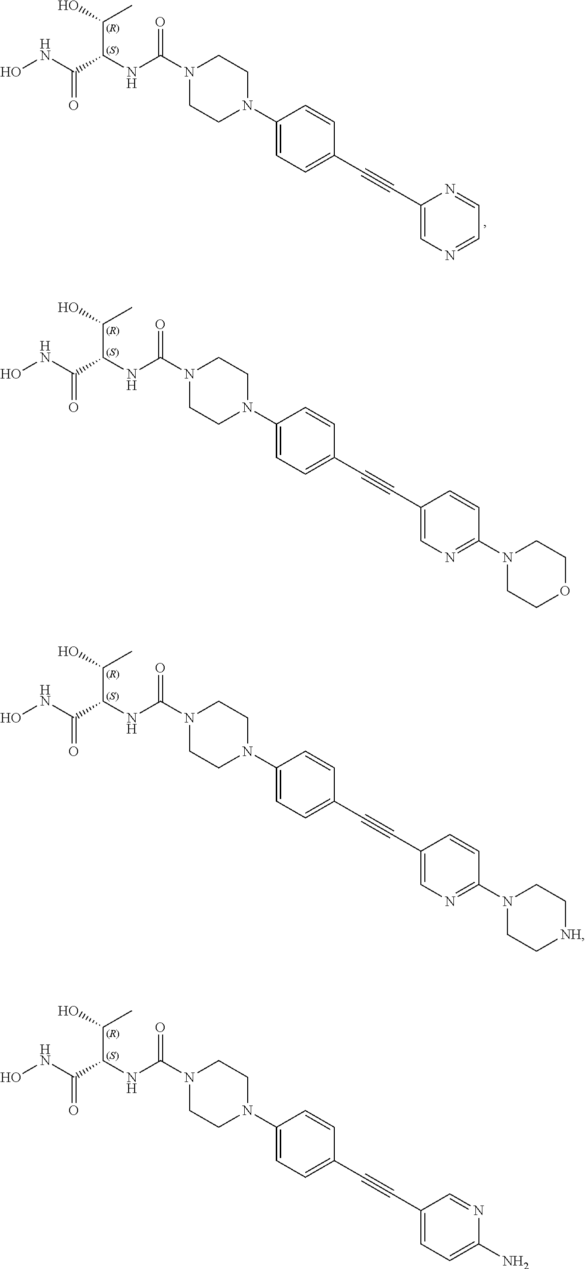

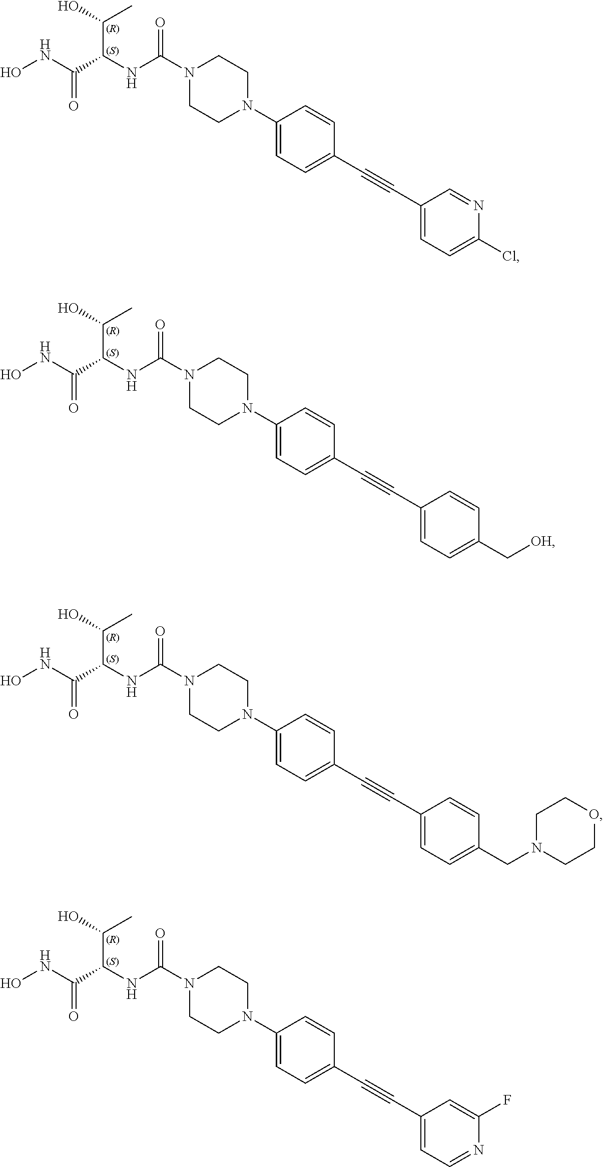

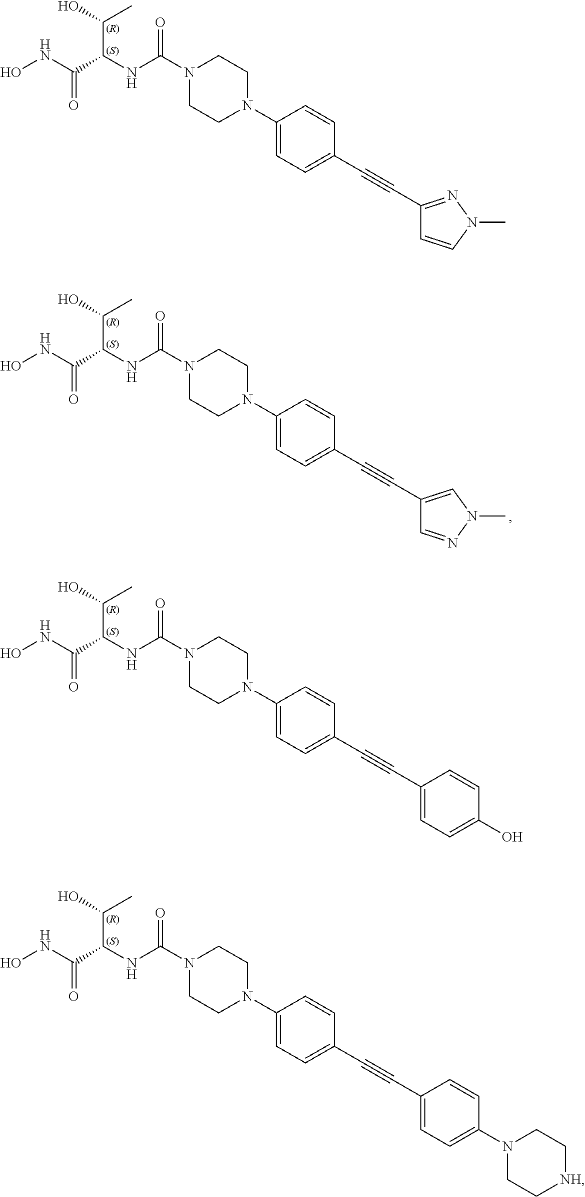

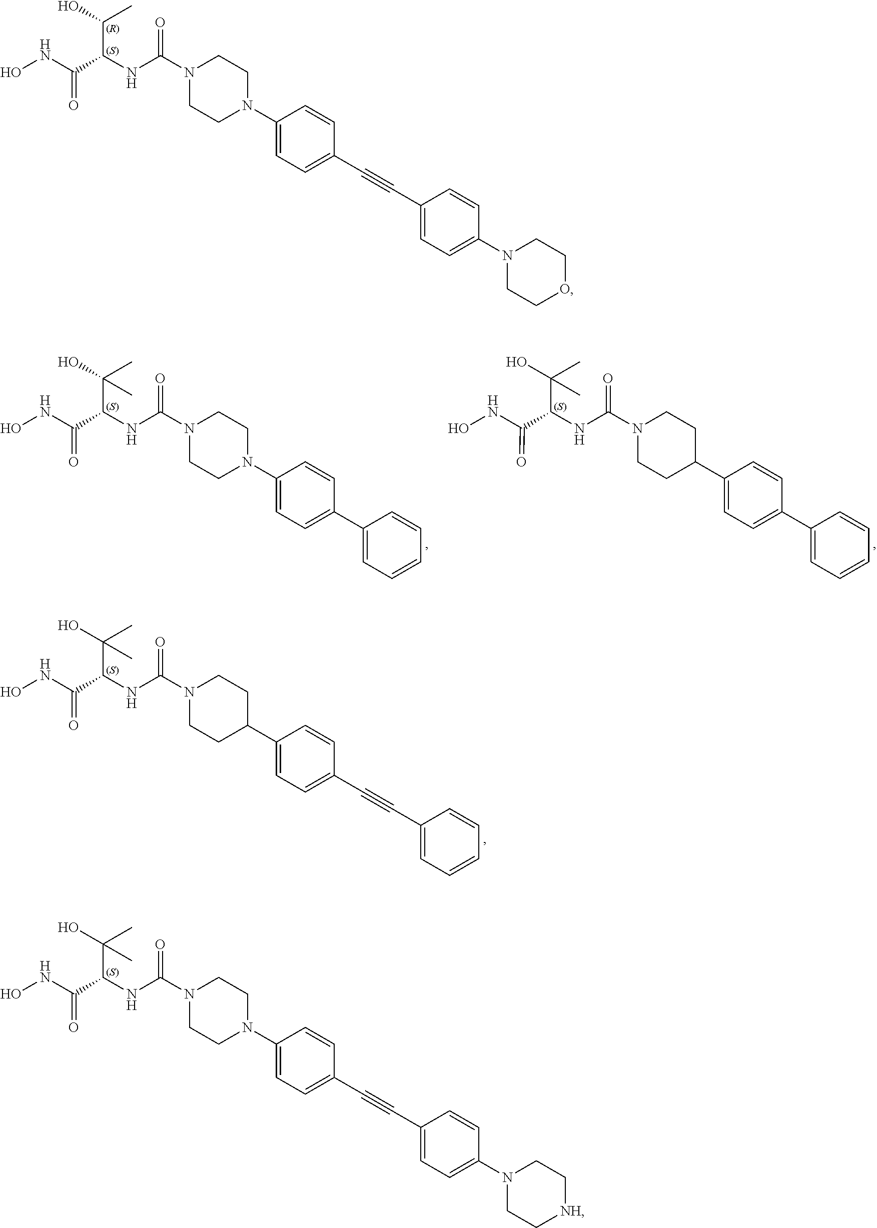

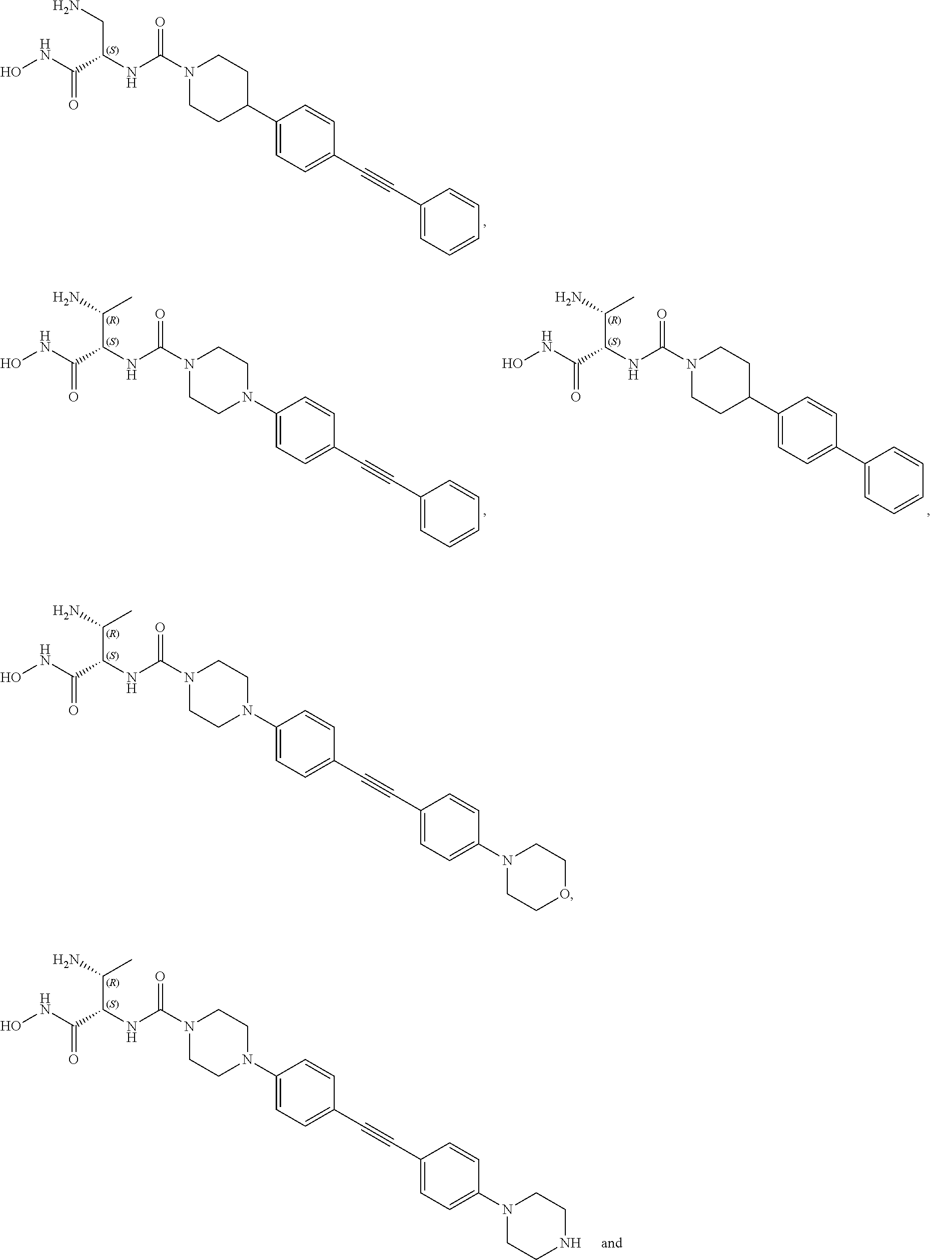

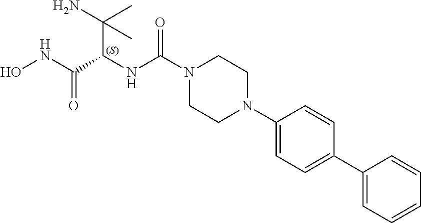

or a pharmaceutically acceptable salt, solvate or ester thereof.

As used above, and throughout this disclosure, the following terms, unless otherwise indicated, shall be understood to have the following meanings:

“Patient//subject” includes both human and animals.

“Mammal” means humans and other mammalian animals.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as mehtyl, ethyl or propyl, are attached to a linear alkyl chain. “Lower alkyl” means a group having about 1 to about 6 carbon atoms in the chain which may be straight or branched. The term “substituted alkyl” means that the alkyl group may be substituted by one or more substituents which may be the same or different, each substituent being independently selected from the group consisting of halo, alkyl, aryl, cycloalkyl, cyano, hydroxy, alkoxy, alkylthio, amino, —NH(alkyl), —NH(cycloalkyl), —N(alkyl)₂, carboxy and —C(O)O-alkyl. Non-limiting examples of suitable alkyl groups include methyl, ethyl, n-propyl, isopropyl and t-butyl. The term “Fluoroalkyl” means an alkyl group in which alkyl is as previously described wherein one or more hydrogens are replaced with fluorine atoms.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. Non-limiting examples of suitable alkenyl groups include ethenyl, propenyl, n-butenyl, 3-methylbut-2-enyl, n-pentenyl, octenyl and decenyl.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. The term “substituted alkynyl” means that the alkynyl group may be substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable muiticyclic cycloalkyls include 1-decalinyl, norbornyl, adamantyl and the like, as well as partially saturated species such as, for example, indanyl, tetrahydronaphthyl and the like.

“Cycloalkenyl” means a non-aromatic mono or multicyclic ring system comprising about 3 to about 10 carbon atoms, preferably about to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Haloalkoxy” means an alkyl as defined above wherein one or more hydrogen atoms on the alkyl is replaced by a halo group defined above. Non-limiting examples include trifluoromethyl, 2,2,2-trifluoroethyl, 2-chloropropyl and alike.

“Haloalkoxy” means an alkoxy group as defined below wherein one or more hydrogen atoms on the alkoxy is replaced by a halo/halogen group defined above. Non-limiting examples include trifluoromethoxy (CF₃O—), difluoromethoxy (CHF₂O—), 2,2,2-trifluoroethoxy (CF₃CH₂O—), 2-chloropropoxy (CH₃CH(Cl)CH₂O—) and alike.

“Halogen” or “halo” means fluorine, chlorine, bromine, or iodine. Preferred are fluorine, chlorine and bromine.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —C(═N—CN)—NH₂, —C(═NH)—NH₂, —C(═NH)—NH(alkyl), Y₁Y₂N—, Y₁Y₂N-alkyl-, Y₁Y₂NC(O)—, Y₁Y₂NSO₂- and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

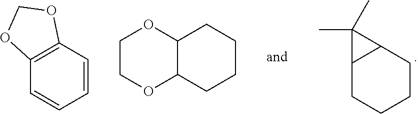

“Heterocyclyl” means a non-aromatic saturated monocyclic or multicyclic ring system comparing about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like.

“Heterocyclenyl” means a partially unsaturated monocyclic or partially unsaturated multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heterocyclenyls contain about 5 to about 6 ring atoms and 1-3 double bonds. Preferred heterocyclenyls also contain at least one —C═N as part of the ring. The “heterocyclenyl” can be optionally substituted by one or more “ring system substituents” which may be the same or different, and are as defined herein. The prefix aza, oxa or thia before the heterocyclenyl root name means that at least a nitrogen, oxygen or sulfur atom respectively, is present as a ring atom. The nitrogen or sulfur atom of the heteroaryl can be optionally oxidized to the corresponding N-oxide, S-oxide or S,S-dioxide. Non-limiting examples of suitable heterocyclenyls include dihydroimidazole, dihydrooxazole, dihydrooxadiazole, dihydrothiazole, and the like.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

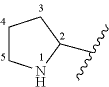

there is no —OH attached directly to carbons marked 2 and 5.

It should also be noted that tautomeric forms such as, for example, the moieties:

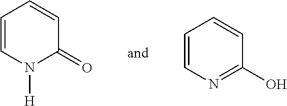

are considered equivalent in certain embodiments of this invention.

“Alkynylalkyl” means an alkynyl-alkyl- group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Aryloxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “isolated” or “in isolated form” for a compound refers to the physical state of said compound after being isolated from a synthetic process or natural source or combination thereof. The term “purified” or “in purified form” for a compound refers to the physical state of said compound after being obtained from a purification process or processes described herein or well known to the skilled artisan, in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form in to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula (I), its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. The term “prodrug”, as employed herein, denotes a compound that is a drug precursor which, upon administration to a subject, undergoes chemical conversion by metabolic or chemical processes to yield a compound of Formula I or a salt and/or solvate thereof. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

“Effective amount” or “therapeutically effective amount” is meant to describe an amount of compound or a composition of the present invention effective in inhibiting the CDK(s) and thus producing the desired therapeutic, ameliorative, inhibitory or preventative effect.

The compounds of Formula I can form salts which are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCR S. Berge et of, Journal of Pharmaceutical Sciences (1977)66(1)1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Compounds of Formula I, and salts, solvates and prodrugs thereof, may exist in their tautomeric form (for example, as an amide or imino ether). All such tautomeric forms are contemplated herein as part of the present invention.

All stereoisomers (for example, geometric isomers, optical isomers and the like) of the present compounds (including those of the salts, solvates and prodrugs of the compounds as well as the salts and solvates of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” “prodrug” and the like, is intended to equally apply to the salt, solvate and prodrug of enantiomers, stereoisomers, rotamers, tautomers, positional isomers, racemates or prodrugs of the inventive compounds.

Polymorphic forms of the compounds of Formula I, and of the salts, solvates and prodrugs of the compounds of Formula I, are intended to be included in the present invention.

The compounds according to the invention have pharmacological properties; in particular, the compounds of Formula I are inhibitors of LpxC.

In one aspect, the invention provides a pharmaceutical composition comprising as an active ingredient at least one compound of formula (I).

In another aspect, the invention provides a pharmaceutical composition of formula (I) additionally comprising at least one pharmaceutically acceptable carrier.

In another aspect, the invention provides a method of treating disorders associated with LpxC, said method comprising administering to a patient in need of such treatment a pharmaceutical composition which comprises a therapeutically effective amount of at least one compound of formula (I).

In another aspect, the invention provides a use of a compound of formula (I) for the manufacture of a medicament to treat disorders associated with LpxC.

The compounds of formula I have antibacterial activity and can be useful in the treatment of a microbial infection, including gram negative and gram positive infections.

In another aspect, the invention provides a method of preparing a pharmaceutical composition for treating the disorders associated with LpxC, said method comprising bringing into intimate contact at least one compound of formula I and at least one pharmaceutically acceptable carrier.

In another aspect, the invention provides a pharmaceutical composition for treating disorders associated with LpxC, in a subject comprising, administering to the subject in need of such treatment a therapeutically effective amount of a compound of formula I or a pharmaceutically acceptable salt, solvate, ester or isomer thereof.

In another aspect, the invention provides a compound of formula I in purified form.

In another aspect, the invention provides a method of treating a condition or disease mediated by LpxC (such as a microbial infection), in a subject comprising: administering to the subject in need of such treatment a therapeutically effective amount of at least one compound of formula I or a pharmaceutical acceptable salt, solvate or isomer thereof.

In another aspect, the invention provides a method for the treatment of a microbial infection in a mammal, comprising administering to said mammal a therapeutically effective amount of a compound of formula I or a pharmaceutically acceptable salt, solvate or ester thereof. In one embodiment, the microbe causing the infection is a bacteria, in another embodiment it is a fungus. In one embodiment, the microbial infection is a gram negative infection; in another embodiment, it is a warn negative infection.

In another aspect, the invention provides a method for the treatment of a microbial infection in a mammal, comprising administering to said mammal a therapeutically effective amount of a compound of formula I in combination with one or more additional antibacterial or antifungal agent. In one embodiment, said additional antibacterial agent is active against gram negative bacteria. In another embodiment, said additional antibacterial agent is active against gram positive bacteria.

In one embodiment, the compounds of Formula (I) can be administered to a subject to treat gram negative bacterial infections. They may also be given along with other antibiotics, such as the macrolides, e.g., erythromycin, rifampicin and azithromycin, to achieve or enhance the gram negative antibacterial activity, or with other non-macrolide antibiotics to achieve or enhance the spectrum or potency of the particular antibacterial agent against gram negative organisms.

Likewise, the compounds of formula I can be used with other agents which are in and of themselves useful in conjunction with antibacterial agents. For example, bacterial cell wall permeabilizing agents can be included. Representative examples of such compounds include EDTA, polymixin B nonapeptide, poly-L-lysine and neomycin. Other permeability enhancing agents known to those skilled in the art can be included herein as well.

In another embodiment, the bacterial infection treatable by the compounds of the present invention is caused by at least one organism selected from the group consisting of Acinetobacter baumannii, Acinetobacter calcoaceticus, Acinetobacter haemolyticus, Acinetobacter hydrophila, Actinobacillus actinomycetemcomitans, Aeromonas hydrophila, Alcaligenes xylosoxidans, Bacteroides distasonis, Bacteroides fragilis, Bacteroides melaninogenicus, Bacteroides ovatus, Bacteroides thetaiotaomicron, Bacteroides vulgatus, Bartonella henselae, Bordetella pertussis, Branhamella catarrhalis, Brucella melitensis, Brucella abortus, Brucella canis, Burkholderia cepacia, Burkholderia mallei, Burkholderia pseudomallei, Campylobacter coli, Campylobacter fetus, Campylobacter jejuni, Citrobacter diversus, Citrobacter freundii, Citrobacter koseri, Coxiella burnetli, Edwarsiella tarda, Ehrilichia chaefeenis, Eikenella corrondens, Enterobacter aerogenes, Enterobacter agglomerans, Enterobacter cloacae, Escherichia coli, Flavobacterium menigosepticum, Francisella tularensis, Fusobacterium spp., Haemophilus ducreyi, Haemophilus influenzae, Haemophilus parainfluenzae, Helicobacter pylori, Kingella kingae, Klebsiella oxytoca, Klebsiella ozaenae, Klebsiella pneumoniae, Klebsiella rhinoscleromatis, Legionella pneumophila, Moraxella catarrhalis, Morganella morganii, Neisseria gonorrhoeae, Neisseria meningitides, Pasteurella multocida, Plesiomonas shigelloides, Porphyromonas asaccharolytica, Porphyromonas gingivalis, Prevotella bivia, Prevotella buccae, Prevotella corporis, Prevotella endodontalis, Prevotella intermedia, Prevotella melaninogenica, Prevotella oralis, Proteus mirabilis, Proteus myxofaciens, Proteus penner, Proteus vulgaris, Providencia alcalifaciens, Providencia rettgeri, Providencia stuarfii, Pseudomonas aeruginosa, Pseudomonas fluorescens, Ricketsia prowozekii, Salmonella enterica, Serratia marcescens, Shigella boydii, Shigella dysenteriae, Shigella flexneri, Shigella sonnei, Stenotrophomonas maltophilia, Streptobacillus moniliformis, Vibrio alginolyticus, Vibrio cholerae, Vibrio parahaemolyticus, Vibrio vuluificus, Yersinia enterocolitica, Yersinia pestis, and Yersinia pseudotuberculosis.

In another embodiment, the bacterial infection is caused by at least one organism selected from the group consisting of Acinetobacter baumannii, Acinetobacter spp., Aeromonas hydrophila, Bacteroides fragilis, Bacteroides spp., Bordetella pertussis, Campylobacter jejuni, Campylobacter spp., Citrobacter freundii, Citrobacter spp., Enterobacter cloacae, Enterobacter spp., Escherichia coli, Fusobacterium spp., Haemophilus influenzae, Haemophilus parainfluenzae, Helicobacter pylori, Klebsiella pneumoniae, Klebsiella spp., Legionella pneumophila, Moraxella catarrhalis, Morganella morganii, Neisseria gonorrhoeae, Neisseria meningitides, Pasteurella multocida, Prevotella spp., Proteus mirabilis, Proteus spp., Providencia stuartii, Pseudomonas aeruginosa, Pseudomonas spp., Salmonella enterica, Salmonella typhi, Serratia marcescens, Shigella spp., Stenotrophomonas maltophilia, Vibrio cholerae, Vibrio spp., and Yersinia spp.

The standard LpxC assay consists of 0.2 nM LpxC enzyme, 1.0 μM UDP-3-O-(R-3-hydroxymyristoyl)-N-acetylglucosamine, and test compound, in assay buffer and 2% DMSO. Assay buffer is comprised of 25 mM HEPES, pH 7.3, 150 mM NaCl, 2.0 mM DTT, and 0.01% BSA. The enzyme reaction is carried out in a 96-well assay plate, in a final volume of 102 μL. Solutions of rest compounds are prepared in 100% DMSO. Reaction additions, in order, are (1) 2.0 μL compound solution, (2) 80 μL of assay buffer, (3) 10 μL of 10 μM UDP-3-O-(R-3-hydroxymyristoyl)-N-acetylgucosamine (in assay buffer) and, (4) 10 μL of LpxC enzyme (20 nM in assay buffer) to initiate the reaction. In positive control reactions, addition (1) has 2.0 μL of 100% DMSO (without compound); these reactions are used as the total signal (TSB) value. Reactions are incubated at room temperature for 60 minutes when 10 μL of 1 N HCl is added to stop the reaction. The plate is shaken by hand for 10 seconds to ensure complete quenching. Assay plates are sealed with foil tape, and stored at −80° C. for 24-48 hr prior to analysis.

The concentrations of substrate and product in the reaction mixtures are determined with BioTrove's proprietary RapidFire™ high-throughput mass spectrometry (HTMS). Assay mixtures are partially purified with reverse phase chromatography, where they are washed with water containing 5 mM ammonium formate and eluted onto the mass spectrometer in 80% acetonitrile, 20% water, and 5 mM ammonium formate. The mass spectrometry peak areas of the substrate and product are measured to determine the concentration of these analytes. The assay signal is the percentage of substrate that is converted to product. Percent inhibition, % I, in test samples is determined from the following equation:

$$\% I = 100 * \frac{( T S B - SampleSignal )}{( T S B )} .$$

Using this method, the following E.coli IC₅₀ (nM) data were obtained for selected Compounds of Formula (I):

Compounds 32, 33, 38, 42, 50, 51, 62, 62, 66, 69, 71, 82, 84-90, 92-97, 103, 105-109, 111-114, 124-126, 141-143, and 151 had an IC₅₀ value of less than about 50 nM.

Compounds 35, 39, 41, 43, 44, 52, 57, 59, 61, 68, 70, 72, 83, 102, 104, 110,127, 132 and 144 had an IC₅₀ value between 50 and 500 nM.

Compounds 34, 36, 37, 45, 53, 64, 65 and 73 had an IC₅₀ value between 500 and 5,000 nM.

Compounds 40, 46 and 58 had an IC₅₀ value between 5,000 and 10,000 nM.

Compounds 60, 63, 67 and 91 had an IC₅₀ value greater than 10,000 nM.

The pharmaceutical compositions containing the active ingredient may be in a form suitable for oral use, for example, as tablets, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for controlled release.

Formulations for oral use may also be presented as hard gelatin capsules wherein the active ingredients is mixed with an inert solid diluent, for example, calcium carbonate, calcium phosphate or kaolin, or a soft gelatin capsules where in the active ingredient is mixed with water or an oil medium, for example peanut oil, liquid paraffin or olive oil.

Aqueous suspensions contain the active material in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example, ethyl or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example, arachis oil, olive oil, sesame oil or coconut oil, or in mineral oil such as liquid paraffin. The oily suspensions may contain a thickening agent, for example, beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, e.g., sweetening, flavoring and coloring agents, may also be present.

The pharmaceutical compositions of the invention may also be in the form of an oil-in-water emulsion. The oily phase may be a vegetable oil, e.g., olive oil or arachis oil, or a mineral oil, e.g., liquid paraffin or mixtures of these. Suitable emulsifying agents may be naturally-occurring phosphatides, e.g., soy beans, lecithin, and esters or partial esters derived from fatty acids and hexitol anhydrides, for example, sorbitan monooleate, and condensation products of the said partial esters with ethylene oxide, e.g., polyoxyethylene sorbitan monooleate. The emulsions may also contain sweetening and flavoring agents.

Syrups and elixirs may be formulated with sweetening agents, for example, glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, e.g., as a solution in 1,3-butane diol. Among the acceptable vehicles and solvents that may be employed are water, Ringer's solution and isotonic sodium chloride solution. In addition, sterile fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of the invention may also be administered in the form of suppositories for rectal administration of the drug. The compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compound of The invention are employed. (For purposes of this application, topical application shall include mouthwashes and gargles.)

The compounds for the present invention can be administered in the intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

The dosage regimen utilizing the compounds of the present invention is selected in accordance with a variety of factors including type, species, weight, sex and medical condition of the patient; the severity of the condition to be treated; the route of administration; the renal and hepatic function of the patient; and the particular compound thereof employed. A physician or veterinarian of ordinary skill can readily determine and prescribe the effective amount of the drug required to prevent, counter, arrest or reverse the progress of the condition. Optimal precision in achieving concentration of drug within the range that yields efficacy without toxicity requires a regimen based on the kinetics of the drug's availability to target sites. This involves a consideration of the distribution, equilibrium, and elimination of a drug. Preferably, doses of the compound of Formula 1 useful in the method of the present invention range from 0.01 to 1000 mg per day. More preferably, dosages range from 0.1 to 1000 mg/day. Most preferably, dosages range from 0.1 to 500 mg/day. For oral administration, the compositions are preferably provided in the form of tablets containing 0.01 to 1000 milligrams of the active ingredient, particularly 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. An effective amount of the drug is ordinarily supplied at a dosage level of from about 0.0002 mg/kg to about 50 mg/kg of body weight per day. The range is more particularly from about 0.001 mg/kg to 1 mg/kg of body weight per day.

Advantageously, the active agent of the present invention may be administered in a single daily dose, or the total daily dosage may be administered in dividend doses of two, the or four time daily.

The amount of active ingredient that may be combined with the carrier materials to produce single dosage form will vary depending upon the host treated and the particular mode of administration.

It will be understood, however, that the specific dose level for any particular patient will depend upon a variety of factors including the age, body weight, general health, sex, diet, time of administration, route or administration, rate of excretion, drug combination and the severity of the particular disease undergoing therapy.

The compounds of the invention may be produced by processes known to those skilled in the art and as shown in the following reaction schemes and in the preparations and examples described below.

### EXAMPLES

The following abbreviations are used in the procedures and schemes:
Anh. Anhydrous
Aq Aqueous
BOC text-Butoxycarbonyl
BSA Bovine Serum Albumin
° C. degrees Celsius
DCM Dichloromethane
DIEA Diisopropylethylamine
DMF Dimethylformamide
DMSO Dimethylsulphoxide
DTT Dithiothreitol
EtOAc Ethyl acetate
g grams
h. hours
¹H proton
HEPES 4-(2-hydroxyethyl)-1-piperazineethanesulfonic acid
HPLC High pressure liquid chromatography
LC-MS Liquid Chromatography-Mass Spectrometry
M Molar
MeCN Acetonitrile
MeOH Methanol
min Minutes
mg Milligrams
MHz Megahertz
ml Milliliter
MS Mass Spectroscopy
RT Room temperature
TFA Trifluoroacetic acid
THF Tetrahydrofuran
TLC Thin layer chromatography
t_{R} Retention time
UV Ultraviolet
X-Phos dicyclohexyl-[2[2,4,6-tri(propan-2-yl)phenyl]phenyl]phosphane

NMR spectra were acquired on a Mercuryplus 400 MHz NMR Spectrometer (Varian), using CDCl3 or DMSO-d6 as solvents. LC-MS data was obtained using an Agilent 1100 Series LC/MSD (quadrupole, API-ES (Atmospheric Pressure Interface Electrospray)) with a capillary voltage set to 3500 V and running in positive mode. Reported analytical HPLC (LC/MS) retention times were obtained using a C18 (150×4.6 mm) reverse-phase column eluting with a 5 or 10 minute gradient of 0.1% trifluoroacetic acid in water to 95:5 acetonitrile:water at a flow rate of 3 mL/min.

Purification via reverse phase chromatography was accomplished using a C18 reverse phase column with a gradient of 0.1% trifluoroacetic acid in water to 95:5 acetonitrile:water at a flow rate of 20 mL/min. Samples were collected using a UV (Gilson, 254 nm) or mass spectra (Agilent 1100 Series LC/MSD model SL) signal.

Normal phase silica gel chromatography on a Biotage instrument was accomplished using a Quad UV System (P/N 07052) utilizing KP-SIL 32-63 um columns, 60 Å with flash cartridges 12+M or 25+M.

The compounds of formula (I) may be produced by processes known to those skilled in the art and as shown in the following reaction schemes and in the preparations and examples described below. These preparations and examples should not be construed to limit the scope of the disclosure. Alternate mechanistic pathways and analogous structures may be apparent to those skilled art. All kinds of isomeric forms of the compounds are considered to be within the scope of this invention.

### Example 1

### Example 1A

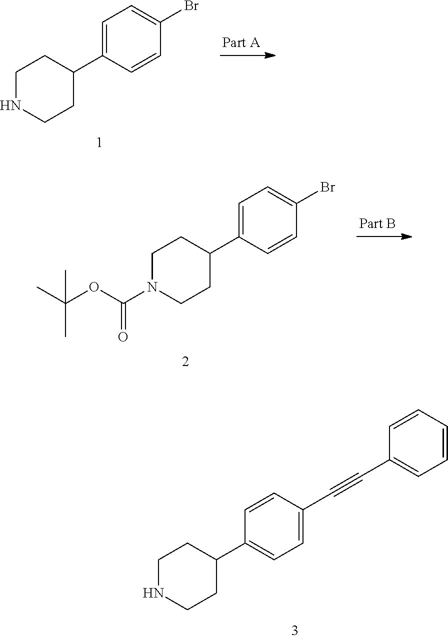

Part A:

A mixture of 4-(4-bromophenyl)piperidine (I) (960 mg, 4.0 mmol) and di-tert-butyl dicarbonate (960 mg, 4.4 mmol) at 0° C., in DCM (10 mL) was warmed to room temperature and stirred for 3 hours. LC-MS analysis indicated the reaction was complete. Dichloromethane (10 mL) was added and the solution washed with 1N HCl (10 mL). Drying over magnesium sulfate, concentration and purification by flash column chromatography, gradient elution (0 to 100%) hexane/ethyl acetate, afforded compound 2 as a white solid (1.36 g, 100% yield). HPLC-MS t_{R}=2.50 min (UV_{254 nm}); mass calculated for formula C₁₆H₂₂BrNO₂ 339.1, observed LCMS m/z 284.1 (M+H−^{t}Bu).

Part B:

A solution of compound 2 (600 mg, 1.76 mmol) in acetonitrile (5 mL) was transferred to a Schlenk tube containing dichlorobis(acetonitrile)palladiurn (II) (4.6 mg, 17.6 μmol), X-Phos (25 mg, 52.9 μmol) and cesium carbonate (1.5 g, 4.59 mmol) and the reaction mixture was stirred at room temperature under an inert atmosphere for 25 minutes. 100 μL of a solution containing phenylacetylene (360 mg, 3.52 mmol) in acetonitrile (2 mL) was added and the reaction mixture heated at 90° C. for 15 minutes. The phenylacetylene solution (100 μL) was added every 15 minutes and the reaction mixture was heated at 90° C. for a total of 2.5 hours. LC-MS analysis indicated the reaction was complete. Water (6 mL) was added and the crude product extracted into ethyl acetate (10 mL). Drying over magnesium sulfate, concentration and purification by flash column chromatography, gradient elution (0 to 100%) hexane/ethyl acetate, afforded BOC-protected compound 3 as a yellow solid (546 mg, 86% yield). HPLC-MS t_{R}=2.70 min (UV_{254 nm}); mass calculated for formula C₂₄H₂₇NO₂ 361.2, observed LCMS m/z 306.2 (M+H−^{t}Bu).

The BOC-protecting group was hydrolyzed by the addition of trifluoroacetic acid (5 mL) and the resulting mixture stirred at room temperature for 1 minute. LC-MS analysis indicated hydrolysis was complete. The volatiles were removed in vacuo and the resulting residue re-dissolved in a 1:1 MeCN/water mixture (10 mL) and lyophilized for 18 hours to afford crude compound 3. HPLC-MS t_{R}=1.22 min (UV_{254 nm}); mass calculated for formula C₁₉H₁₉N 261.2, observed LCMS m/z 262.2 (M+H).

### Example 1B

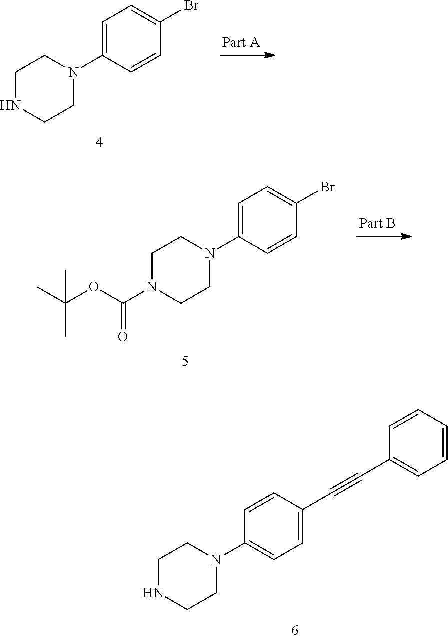

Compound 6 was prepared from 1-(4-bromophenyl)piperazine (4) using the conditions described in Example 1A, Part A and Part B. HPLC-MS t_{R}=1.19 min (UV_{254 nm}); mass calculated for formula C₁₈H₁₈N₂ 262.2, observed LCMS m/z 263.1 (M+H).

### Example 1C

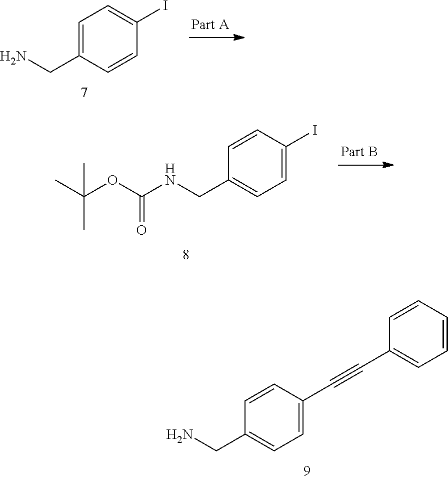

Part A:

Compound 8 was prepared from 4-iodobenzylamine (7) using the conditions described in Example 1A, Part A. HPLC-MS t_{R}=2.15 min (UV_{254 nm}); mass calculated for formula C₁₁H₁₄INO₂ 333.10, observed LCMS m/z 278.1 (M+H−^{t}Bu).

Part B:

To a mixture of compound 8 (333 mg, 1.0 mmol), copper iodide (3.8 mg, 0.02 mmol) and dichlorobis(triphenylphosphine)palladium (II) (7.0 mg, 0.01 mmol) in THF (5 mL) was added phenylacetylene (122 mg, 1.2 mmol) and triethylamine (298 μL, 2 mmol). The reaction vessel was flushed with argon, and the reaction mixture stirred at room temperature for 18 hours. LC-MS analysis of the reaction indicated that the reaction was complete. Ethyl acetate (5 mL) was added and the reaction mixture washed with saturated NaHCO₃. Drying over magnesium sulfate, concentration and purification by flash column chromatography, gradient elution (0 to 100%) hexane ethyl acetate, afforded BOC-protected compound 9 as a yellow solid (258 mg, 84% yield). HPLC-MS t_{R}=2.23 min (UV_{254 nm}); mass calculated for formula C₂₀H₂₁NO₂ 307.2, observed LCMS m/z 330.1 (M+Na).

The BOC-protecting group was hydrolyzed by the addition of trifluoroacetic acid (5 mL) and the resulting mixture stirred at room temperature for 1 minute. LC-MS analysis indicated hydrolysis was complete. The volatiles were removed in vacuo and the resulting residue re-dissolved in a 1:1 MeCN/water mixture (10 mL) and lyophilized for 18 hours to afford crude compound 9. HPLC-MS t_{R}=1.11 min (UV_{254 nm}); mass calculated for formula C₁₅H₁₃N 207.2, observed LCMS m/z 208.2 (M+H).

### Example 1D

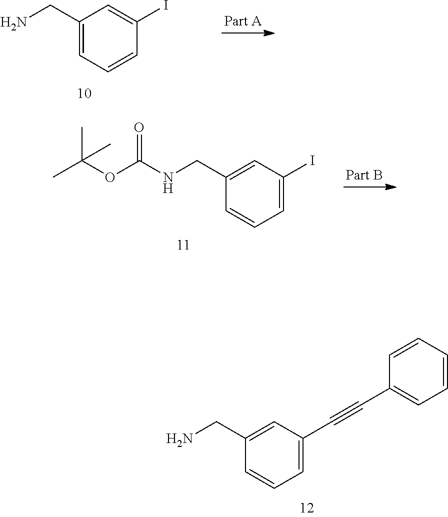

Compound 12 was prepared from 3-iodobenzylamine (10) using the conditions described in Example 1C, Part A and Part B. HPLC-MS t_{R}=1.03 min (UV_{254 nm}); mass calculated for formula C₁₅H₁₃N 207.2, observed LCMS m/z 208.2 (M+H).

### Example 1E

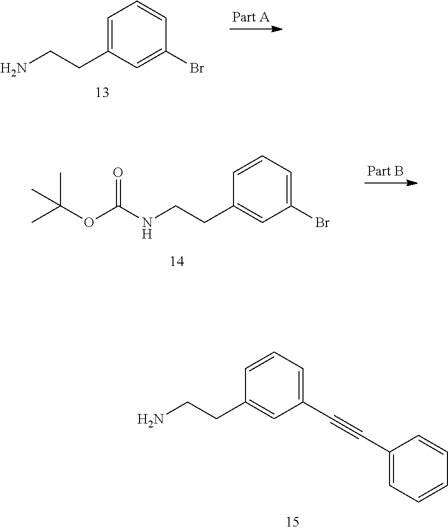

Compound 15 was prepared from 3-bromophenethylamine using the conditions described in Example 1A, Part A and Part B. HPLC-MS t_{R}=1.18 min (UV_{254 nm}); mass calculated for formula C₁₆H₁₅N 221.1, observed LCMS m/z 222.1 (M+H).

### Example 1F

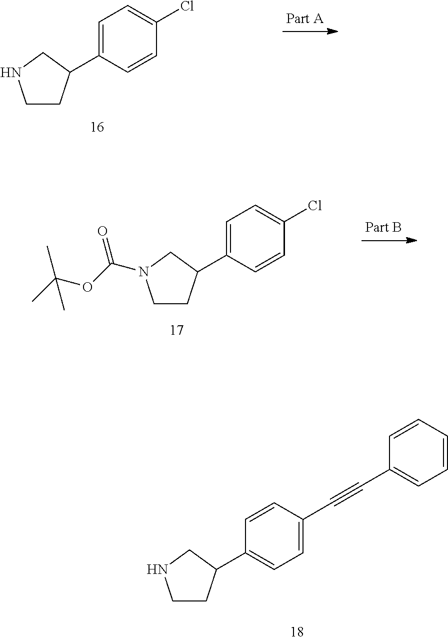

Compound 18 was prepared from 3-(4-chlorophenyl)pyrrolicline (16) using the conditions described in Example 1A, Part A and Part B. HPLC-MS t_{R}=1.27 min (UV_{254 nm}); mass calculated for formula C₁₈H₁₇N 247.1, observed LCMS m/z 248.1 (M+H).

### Example 1G

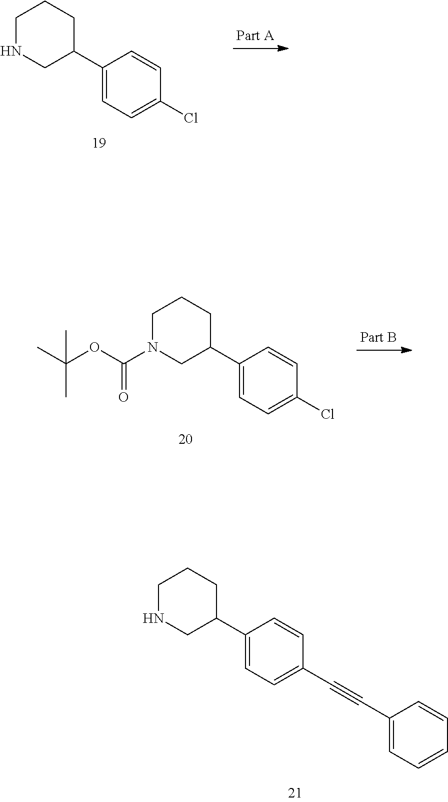

Compound 21 was prepared from 3-(4-chlorophenyl)piperidine (19) using the conditions described in Example 1A, Part A and Part B. HPLC-MS t_{R}=1.28 min (UV_{254 nm}); mass calculated for formula C₁₉H₁₉N 261.2, observed LCMS m/z 262.2 (M+H).

### Example 1H

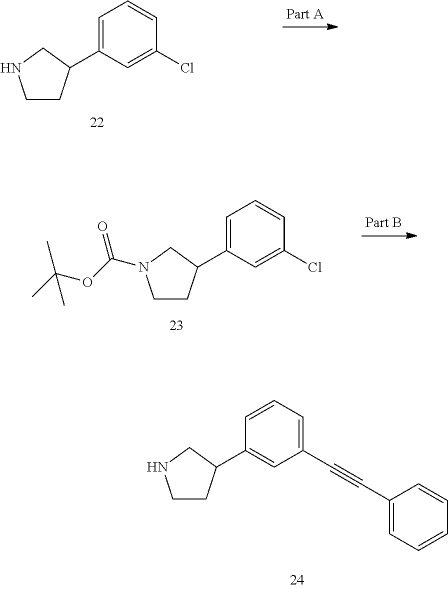

Compound 24 was prepared from 3-(3-chlorophenyl)pyrrolidine (22) using the conditions described in Example 1A, Part A and Part B. HPLC-MS t_{R}=1.20 min (UV_{254 nm}); mass calculated for formula C₁₈H₁₇N 247.1, observed LCMS m/z 248.1 (M+H).

### Example 1I

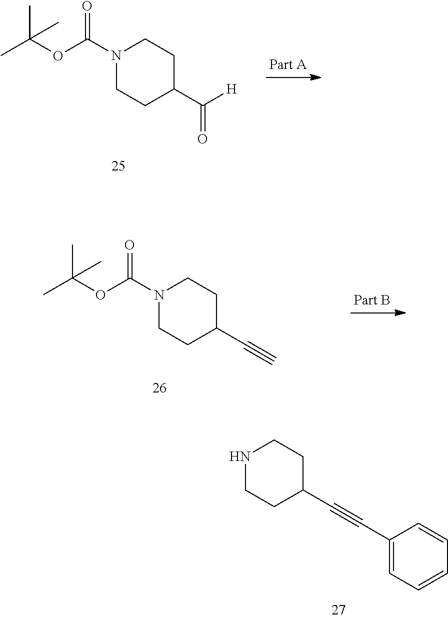

Part A:

Compound 26 was prepared form 1-N-boc-4-formylpiperidine (25) according to reference J. Med. Chem. (2004), 47, 12, 3111.

Part B:

Compound 27 was prepared from compound 26 and iodobenzene using the Sonagashira coupling conditions described in Example 1C, Part B. HPLC-MS t_{R}=0.85 min (UV_{254 nm}); mass calculated for formula C₁₃H₁₅N 185.1, observed LCMS m/z 186.1 (M+H).

### Example 2

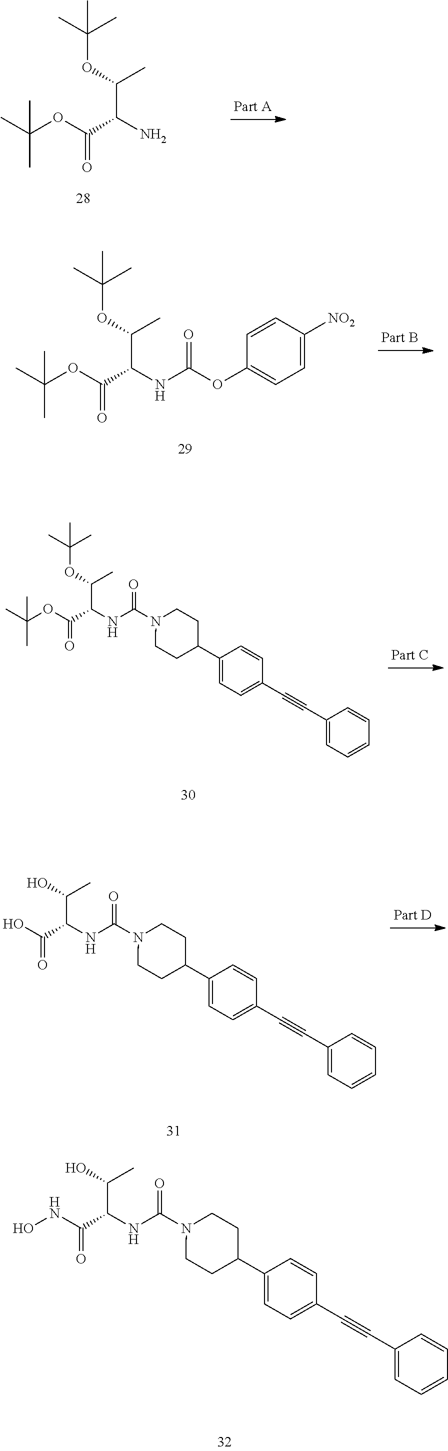

Part A:

To an ice-cooled solution of 4-nitrophenyl chloroformate (665 mg, 3.3 mmol) and DMA (1.6 mL, 9 mmol) in THF (10 mL) was slowly added over 20 minutes a solution of O-tert-butyl-L-threonine tert-butyl ester hydrochloride (28) (803 mg, 3 mmol) in THF (5 mL). The reaction mixture was warmed to room temperature and stirred for 18 hours. LC-MS analysis indicated the reaction was complete. The reaction was quenched with the addition of saturated NaHCO₃ and extracted with EtOAc. Drying over magnesium sulfate and concentration afforded compound 29 which was subjected to flash silica chromatography, gradient elution (0 to 100%) hexane/ethyl acetate (917 mg, 77%). HPLC-MS t_{R}=2.22 min (UV254 nm); mass calculated for formula C₁₉H₂₈N₂O₇ 396.2 observed LCMS m/z 397.1 (M+H).

Part B:

To a solution of compound 29 (291 mg, 0.72 mmol) and DIEA (0.21 mL, 1.2 mmol) in THF (5 mL) was added amine building block (164 mg, 0.6 mmol) and the reaction mixture heated at 80° C. for 2 hours. The reaction was quenched with the addition of 1N HCl and extracted with EtOAc. Drying over magnesium sulfate and concentration afforded compound 30 which was subjected to flash silica chromatography, gradient elution (0 to 100%) hexane/ethyl acetate (300 mg, 92%). HPLC-MS t_{R}=2.82 min (UV₂₅₄ nm); mass calculated for formula C₃₂H₄₂N₂O₄ 518.3, observed LCMS m/z 519.2 (M+H).

Part C:

Trifluoroacetic acid (5 mL) was added to compound 30 and the resulting mixture stirred at room temperature for 1 hour. LC-MS analysis indicated hydrolysis was complete. The volatiles were removed in vacuo and the resulting residue re-dissolved in a 1:1 MeCN/water mixture (10 mL) and lyophilized for 18 hours to afford crude compound 31. HPLC-MS t_{R}=1.94 min (UV_{254 nm}); mass calculated for formula C₂₄H₂₆N₂O₄ 406.2, observed LCMS m/z 407.1 (M+H).

Part D:

To a solution of compound 31 (15 mg, 0.037 mmol) and HATU (17 mg, 0.044 mmol) in DMF (2 mL) was added DIEA (19 μL, 0.11 mmol) and O-(tert-butyldimethylsilyl)hydroxylamine (6.5 mg, 0.044 mmol). The reaction mixture was stirred at room temperature for 18 hours. LC-MS analysis indicated the reaction was complete. The volatiles were removed in vacuo and the resulting residue purified by Prep.HPLC to afford compound 32 (9.2 mg, 60%) as an off white solid.

The compounds 32-46 (Table-1) were synthesized using the procedure described in the example 2:

TABLE-1

| | | | | Ret. |
|---|---|---|---|---|
| Compound | | Exact | MS m/z | Time |
| Number | Structure | mass | (M⁺ + H) | (min) |
| 32 | 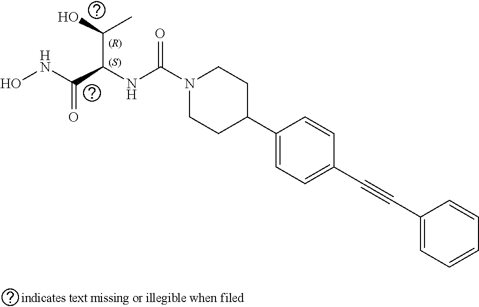 | 421.2 | 422.1 | 4.21 |
| 33 | 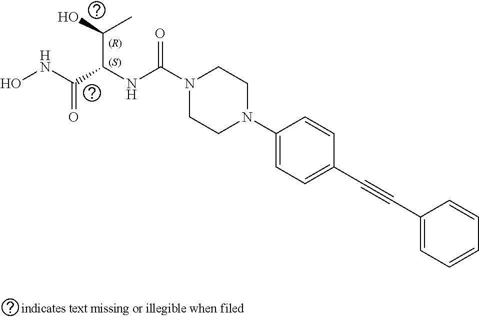 | 422.2 | 423.1 | 3.89 |
| 34 | 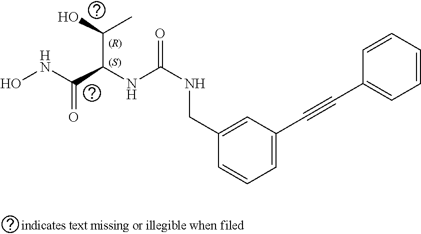 | 367.2 | 368.1 | 3.62 |
| 35 | 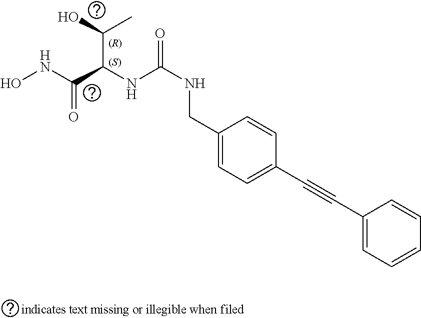 | 367.2 | 368.1 | 3.63 |
| 36 | 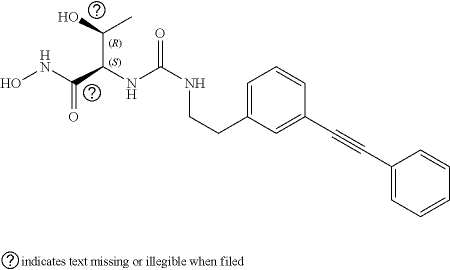 | 381.2 | 382.2 | 3.83 |
| 37 | 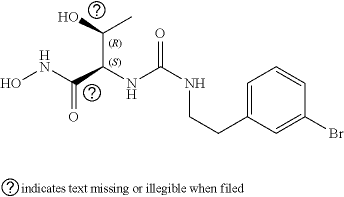 | 359.0 | 360.1 | 2.66 |
| 38 | 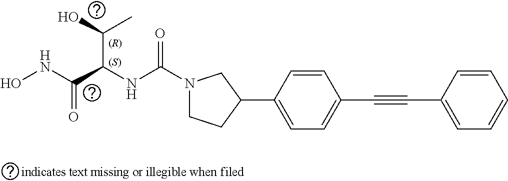 | 407.2 | 408.1 | 4.12 |
| 39 | 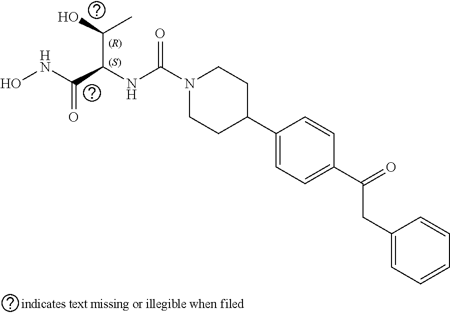 | 439.2 | 440.1 | 3.54 |
| 40 | 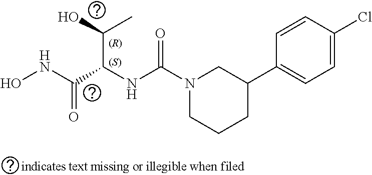 | 355.1 | 356.1 | 3.29 |
| 41 | 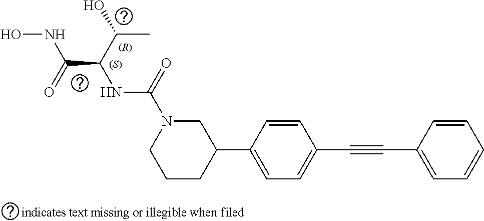 | 421.2 | 422.1 | 4.39 |
| 42 | 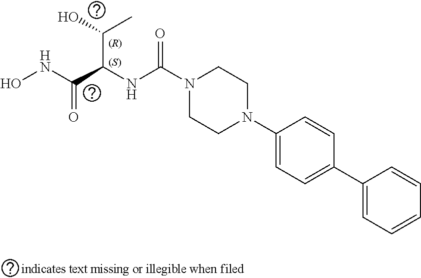 | 398.2 | 399.1 | 3.15 |
| 43 | 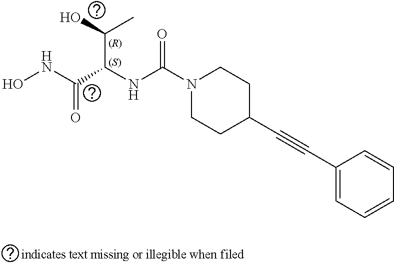 | 345.2 | 346.2 | 3.18 |
| 44 | 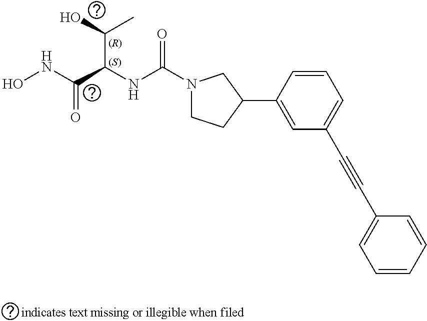 | 407.2 | 408.1 | 4.09 |
| 45 | 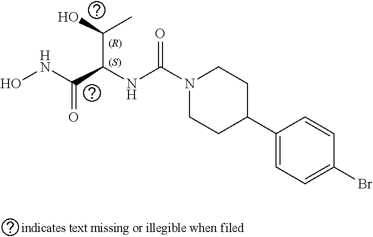 | 399.1 | 400.0 | 3.50 |
| 46 | 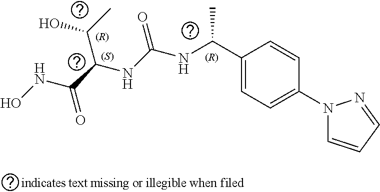 | 347.2 | 348.1 | 2.33 |

### Example 3

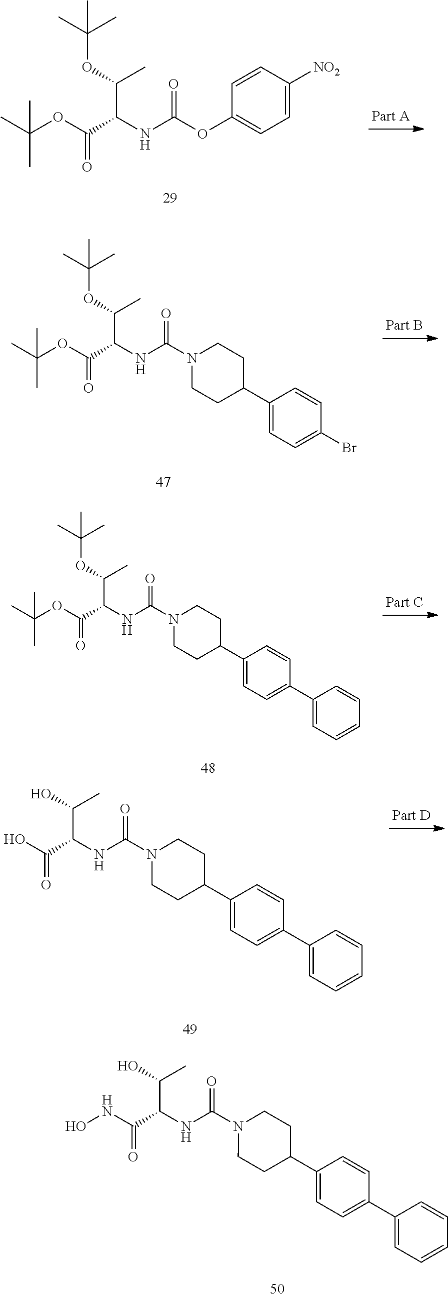

Part A:

Compound 47 (280 mg, 68%) was prepared from the reaction of compound 29 (363 mg, 0.92 mmol) with 1-(4-bromophenyl)piperidine using the conditions described in Example 2, Part B. HPLC-MS t_{R}=2.32 min (UV_{254 nm}); mass calculated for formula C₂₄H₃₇BrN₂O₄ 496.2, observed LCMS 497.2 (M+H).

Part B:

To a mixture of compound 47 (71 mg, 0.14 mmol), potassium phosphate (91 mg, 0.43 mmol) and dichloro[1,1′-bis(diphenylphosphino)ferrocene]palladium (II) dichloromethane adduct (10.5 mg, 0.014 mmol) in dioxane (2 mL) was added phenylboronic acid (34 mg, 0.28 mmol). The reaction vessel was flushed with argon, and the reaction mixture heated at 80° C. for 18 hours. LC-MS analysis of the reaction indicated that the reaction was complete. Ethyl acetate (5 mL) was added, and the precipitates removed by passing through a plug of celite. The filtrate was concentrated, and the crude residue purified by flash column chromatography, gradient elution (0 to 100%) hexane/ethyl acetate, to afford compound 48 as a white solid (61 mg, 86% yield). HPLC-MS t_{R}×2.38 min (UV_{254 nm}); mass calculated for formula C₃₀H₄₂N₂O₄ 494.3, observed LCMS m/z 495.2 (M+H).

Part C:

Compound 49 was prepared from compound 48 using the hydrolysis conditions described in Example 2, Part C. HPLC-MS t_{R}=1.55 min (UV_{254 nm}); mass calculated for formula C₂₁H₂₅N₃O₄ 383.2, observed LCMS m/z 384.2 (M+H).

Part D:

Compound 50 was prepared from compound 49 using the peptide coupling conditions described in Example 2, Part D.

The compounds 50-53 (Table-2) synthesized using the procedure described in example 3:

TABLE-2

| | | | | Ret. |
|---|---|---|---|---|
| Compound | | Exact | MS m/z | Time |
| Number | Structure | mass | (M⁺ + H) | (min) |
| 50 | 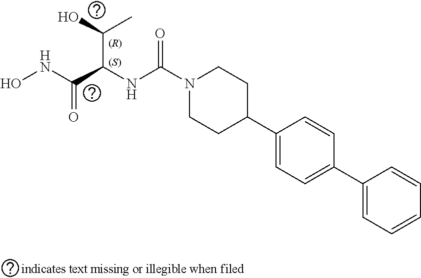 | 397.2 | 398.2 | 3.88 |
| 51 | 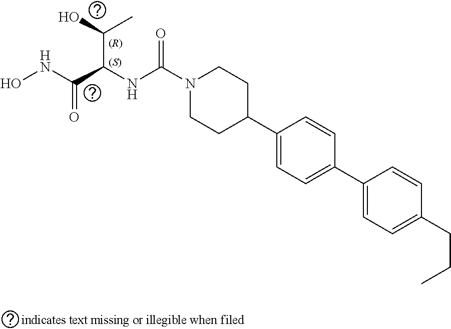 | 439.2 | 440.2 | 4.88 |
| 52 | 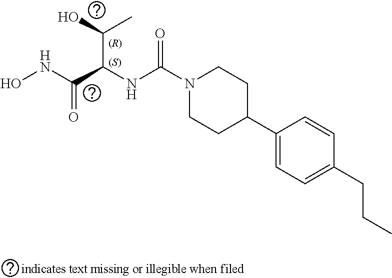 | 363.2 | 364.2 | 3.90 |
| 53 | 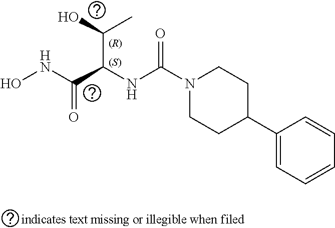 | 321.2 | 322.1 | 2.70 |

### Example 4

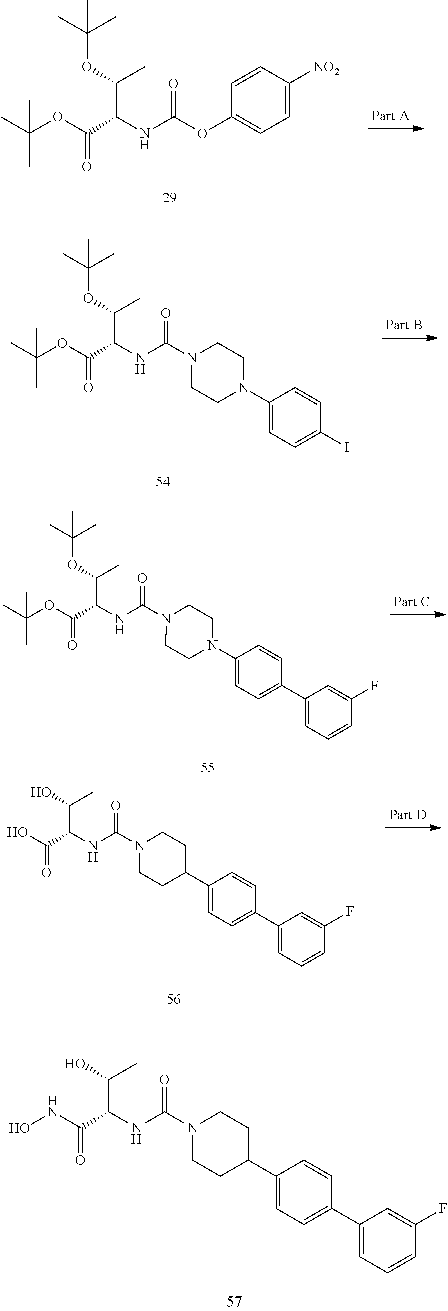

Part A:

Compound 54 (130 mg, 95%) was prepared from the reaction of compound 29 (100 mg, 0.25 mmol) with 1-(4-iodophenyl)piperidine using the conditions described in Example 2, Part B. HPLC-MS t_{R}=2.50 min (UV_{254 nm}); mass calculated for formula C₂₄H₃₇IN₃O₄ 544.2, observed LCMS m/z 545.2 (M+H).

Part B:

Compound 55 (40 mg, 85%) was prepared from the reaction of compound 54 (50 mg, 0.092 mmol) with 3-fluorophenylboronic acid using the conditions described in Example 3, Part B. HPLC-MS t_{R}=2.43 min (UV_{254 nm}); mass calculated for formula C₂₉H₄₀FN₃O₄ 513.3, observed LCMS m/z, 514.3 (M+H).

Part C:

Compound 56 was prepared from compound 55 using the hydrolysis conditions described in Example 2. Part C. HPLC-MS t_{R}=1.60 min (UV_{254 nm}); mass calculated for formula C₂₁H₂₄FN₃O₄ 401.2, observed LCMS m/z 402.2 (M+H).

Part D:

Compound 57 was prepared from compound 56 using the peptide coupling conditions described in Example 2, Part D.

The compounds 57-73 (Table-3) were synthesized as exemplified in the procedure example 4:

TABLE-3

| Com- | | | | Ret. |
|---|---|---|---|---|
| pound | | Exact | MS m/z | Time |
| Number | Structure | mass | (M⁺ + H) | (min) |
| 57 | 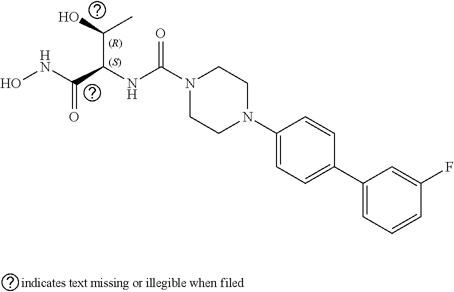 | 416.2 | 417.2 | 3.26 |
| 58 | 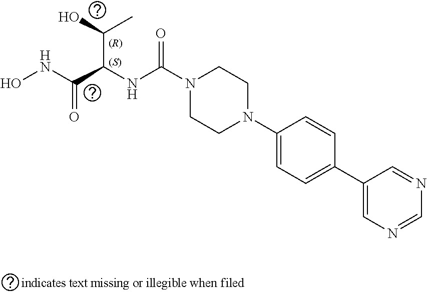 | 400.2 | 401.2 | 2.00 |
| 59 | 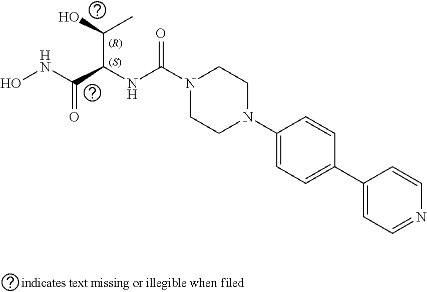 | 399.2 | 400.2 | 1.62 |
| 60 | 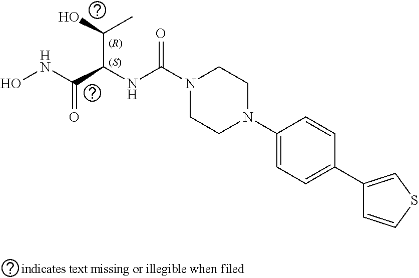 | 404.2 | 405.2 | 2.74 |
| 61 | 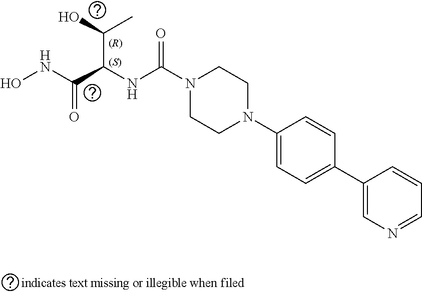 | 399.2 | 400.2 | 1.53 |
| 62 | 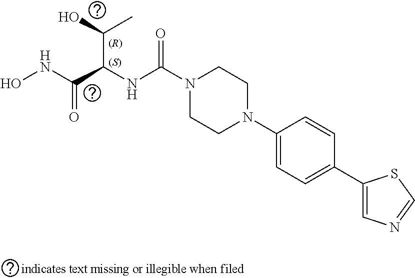 | 405.2 | 406.2 | 2.08 |
| 63 | 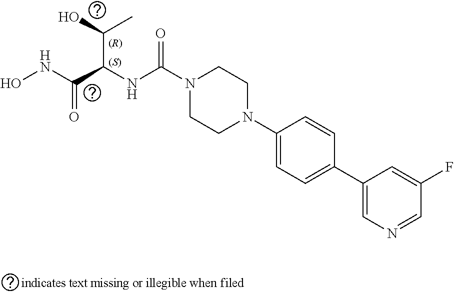 | 417.2 | 418.2 | 2.15 |
| 64 | 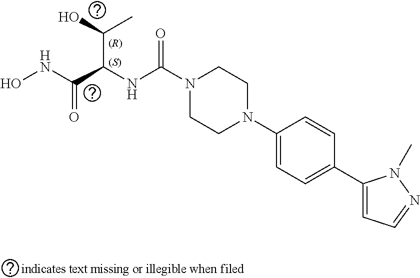 | 402.2 | 403.2 | 2.04 |
| 65 | 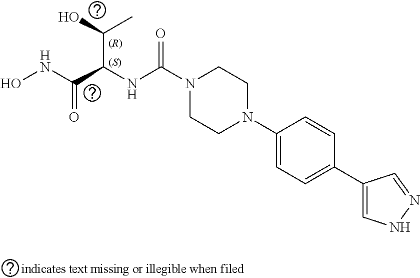 | 388.2 | 389.2 | 1.39 |
| 66 | 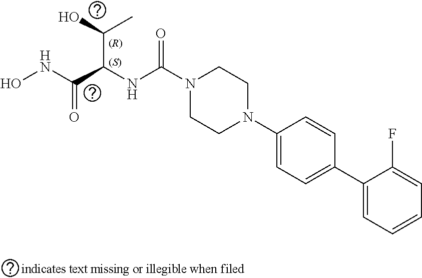 | 416.2 | 417.2 | 3.08 |
| 67 | 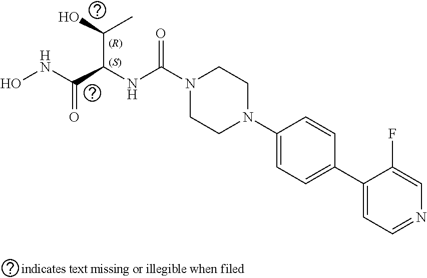 | 417.2 | 418.2 | 1.83 |
| 68 | 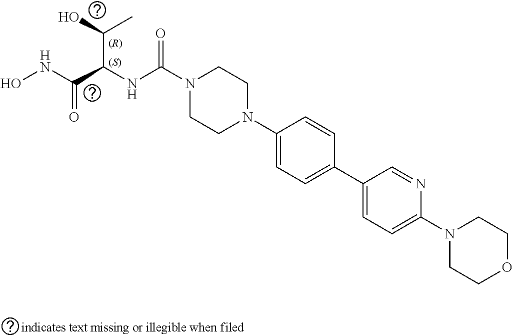 | 484.2 | 485.2 | 1.83 |
| 69 | 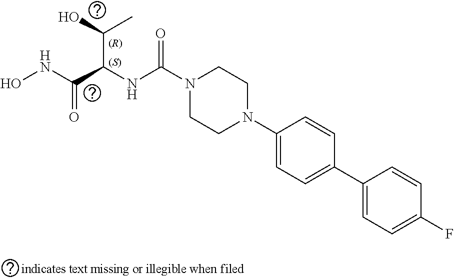 | 416.2 | 417.2 | 3.11 |
| 70 | 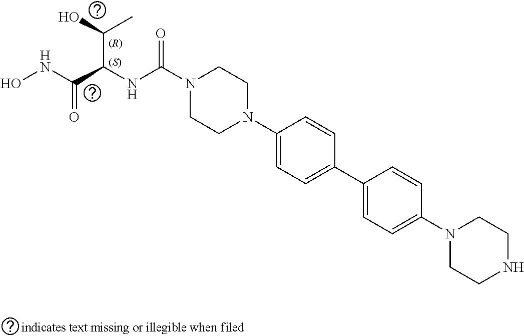 | 482.3 | 483.3 | 1.76 |
| 71 | 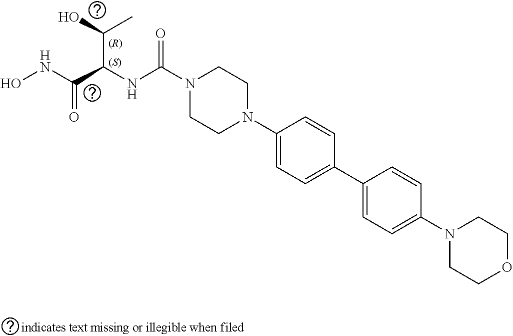 | 483.3 | 484.3 | 2.37 |
| 72 | 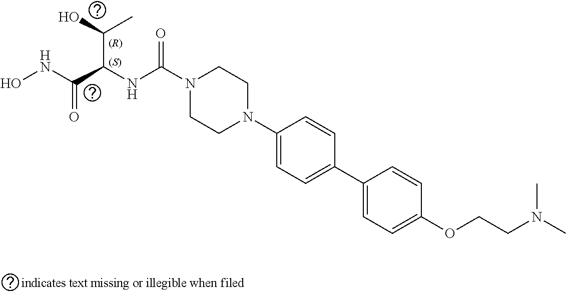 | 485.3 | 486.3 | 1.89 |
| 73 | 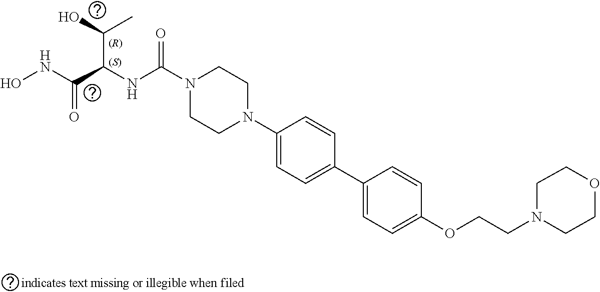 | 527.3 | 528.3 | 1.91 |

### Example 5

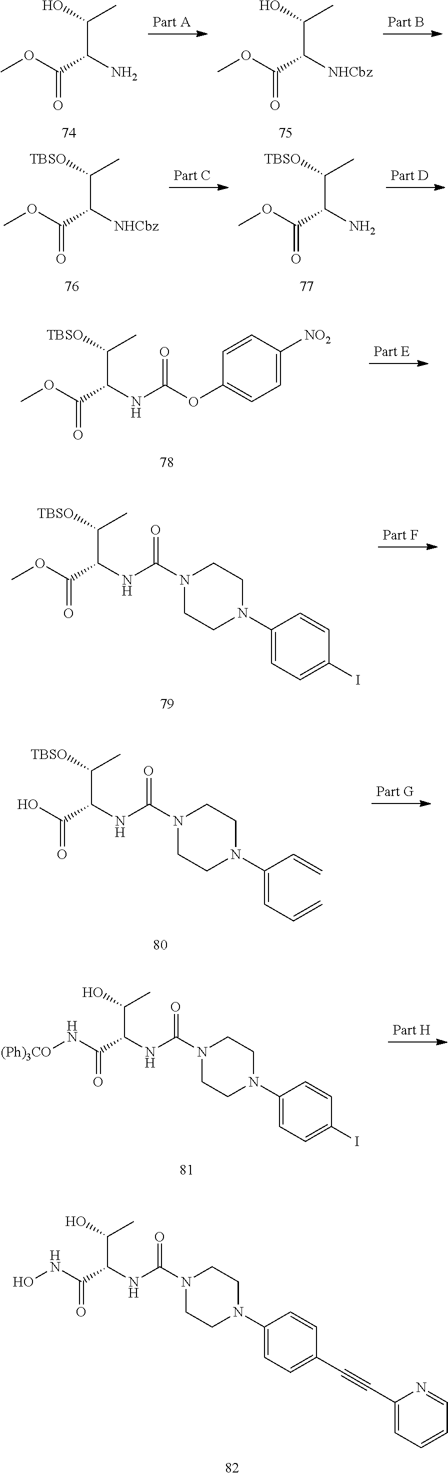

Part A:

To an ice-cooled solution of L-threonine methyl ester hydrochloride (74) (1.7 g, 10 mmol) and DIEA (3.83 mL, 22 mmol) in THF (20 mL) was slowly added over 5 minutes a solution of benzyl chloroformate (1.55 mL, 11 mmol) in THF (10 mL). The reaction mixture was warmed to room temperature and stirred for 2 hours. LC-MS analysis indicated the reaction was complete. The reaction was quenched with the addition of 1N HCl and extracted with EtOAc. Drying over magnesium sulfate and concentration. afforded crude compound 75 as a white solid (2.67 g, 100%). HPLC-MS t_{R}=1.31 min (UV_{254 nm}); mass calculated for formula C₁₃H₁₇NO₅ 267.1, observed LCMS m/z 268.1 (M+H).

Part B:

A solution of compound 75 (561 mg, 2.1 mmol), imidazole (172 mg, 2.5 mmol) and tert-butyldimethylsilyl chloride (348 mg, 2.3 mmol) in DMF (5 mL) was stirred at room. temperature for 18 hours. LC-MS analysis indicated the reaction was complete. The reaction was quenched with water and extracted with EtOAc. Drying over magnesium sulfate and concentration afforded crude compound 76 as a colorless oil (759 mg, 95%). HPLC-MS t_{R}=2.70 min (UV^{254 nm}); mass calculated for formula C₁₉H₃₁NO₅Si 381.2 observed LCMS m/z 382.1 (M+H).

Part C:

A solution of compound 76 (759 mg, 2 mmol) and palladium on charcoal (10%) in EtOAc (10 mL) was subjected to hydrogenation for 18 hours. LC-MS analysis indicated the reaction was complete. The reaction mixture was filtered by passing through celite, and evaporated to afford crude compound 77 as a colorless oil (400 mg, 81%). HPLC-MS t_{R}=1.15 min (UV_{254 nm}); mass calculated for formula C₁₁H₂₅NO₃Si 247.2, observed LCMS m/z 248.1 (M+H).

Part D:

Compound 78 (505 mg, 76%) was prepared from compound 77 (400 mg, 1.62 mmol) using the conditions described in Example, Part A. HPLC-MS t_{R}=2.65 min (UV_{254 nm}); mass calculated for formula C₁₈H₂₈N₂O₇Si 412.2, observed LCMS m/z 413.2 (M+H).

Part E:

Compound 79 (542 mg, 79%) prepared from the reaction of compound 78 (505 mg, 1.23 mmol) with 1-(4-iodophenyl)piperazine hydrochloride using the conditions described in Example 2, Part B. HPLC-MS t_{R}=2.80 min (UV_{254 nm}); mass calculated for formula C₂₂H₃₆IN₃O₄Si 561.2, observed LCMS m/z 562.2 (M+H).

Part F:

A solution containing compound 79 (550 mg, 0.98 mmol) and lithium hydroxide (1M, 1.2 mL, 1.2 mmol) in THF (10 mL) and water (5 mL) was heated at 55° C. for 2 hours. LC-MS analysis indicated that the hydrolysis was complete. The reaction mixture was acidified to pH 4.0 with 1N HCl, and the crude product extracted into EtOAc (2×10 mL). Drying over magnesium sulfate and concentration afforded compound 80 as a yellow solid (506 mg, 94%). HPLC-MS t_{R}=2.60 min (UV_{254 nm}); mass calculated for formula C₂₁H₃₄IN₃O₄Si 547.1, observed LCMS m/z 548.1 (M+H).

Part G:

Compound 81 (400 mg, 63%) was prepared from the reaction of compound 80 (506 mg, 0.93 mmol) with O-tritylhydroxylamine using the peptide coupling conditions described in Example 2, Part D. The O-tert-butyldimethylsilyl protecting group was also hydrolyzed under these conditions. HPLC-MS t_{R}=2.29 min (UV_{254 nm}); mass calculated for formula C₃₄H₃₅IN₄O₄ 690.2, observed LCMS m/z 691.1 (M+H).

Part H:

To a mixture of compound 81 (30 mg, 0.043 mmol), copper iodide (0.57 mg, 0.003 mmol) and dichlorobis(triphenylphosphine)palladium (II) (1.06 mg, 0.0015 mmol) in THF (2 mL) was added 2-ethynylpyridine (6.73 mg, 0.065 mmol) and triethylamine (14 μL, 0.1 mmol). The reaction vessel was flushed with argon, and the reaction mixture stirred at room temperature for 18 hours. LC-MS analysis of the reaction indicated that the reaction was complete. Ethyl acetate (5 mL) was added, and the precipitates removed by passing through a plug of celite. The filtrate was concentrated, and the crude residue subjected to acid hydrolysis using 5% trifluoroacetic acid in DCM (3 mL) for 5 minutes at room temperature. Full hydrolysis was confirmed by LC-MS analysis. The volatiles were removed in vacuo and the resulting residue purified by Prep.HPLC to afford compound 82 as an off white solid.

Compounds from 87-97 (Table-4) were synthesized utilizing the procedure described in the example 5:

TABLE-4

| | | | | Ret. |
|---|---|---|---|---|
| Compound | | Exact | MS m/z | Time |
| Number | Structure | mass | (M⁺ + H) | (min) |
| 82 | 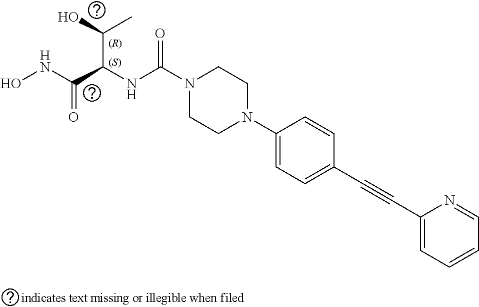 | 423.2 | 424.1 | 2.18 |
| 83 | 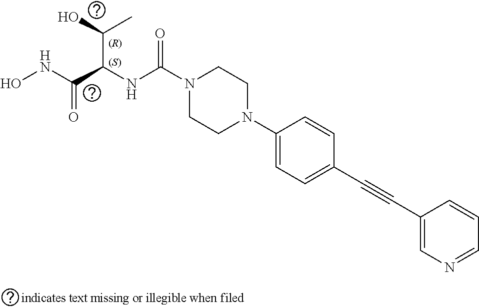 | 423.2 | 424.1 | 2.22 |
| 84 | 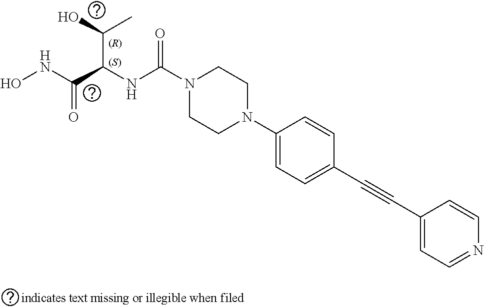 | 423.2 | 424.1 | 2.01 |
| 85 | 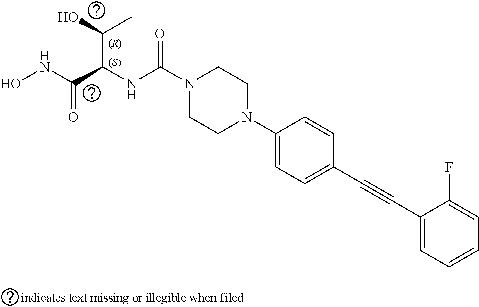 | 440.2 | 441.2 | 3.74 |
| 86 | 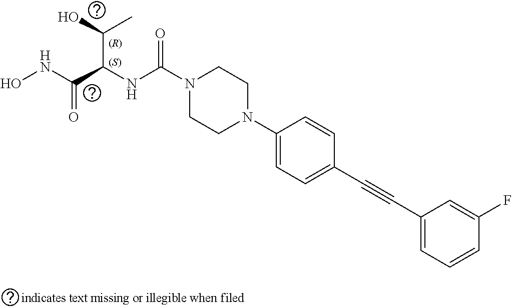 | 440.2 | 441.2 | 3.86 |
| 87 | 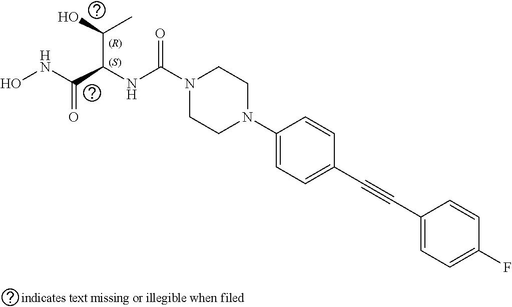 | 440.2 | 441.2 | 3.80 |
| 88 | 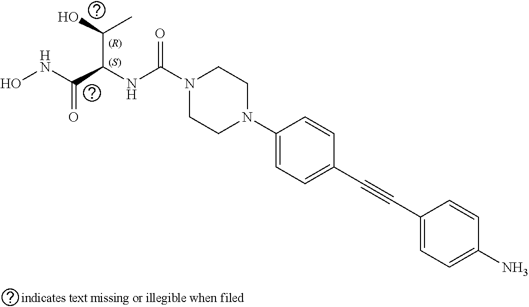 | 437.2 | 438.2 | 2.22 |
| 89 | 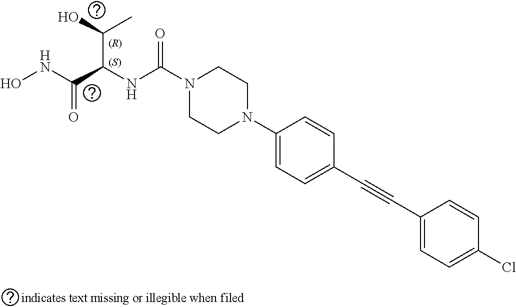 | 456.2 | 457.2 | 4.19 |
| 90 | 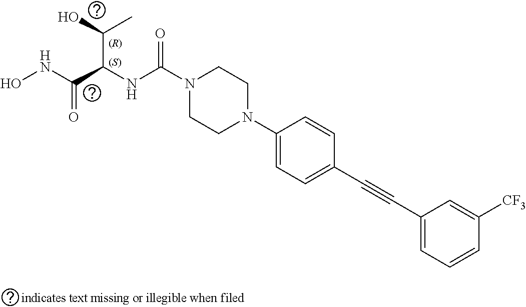 | 490.2 | 491.2 | 4.30 |
| 91 | 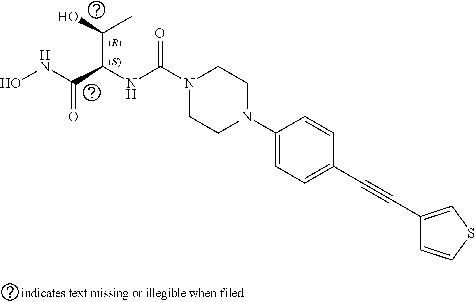 | 428.2 | 429.1 | 3.50 |
| 92 | 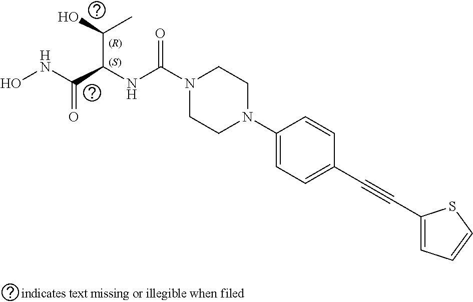 | 428.2 | 429.1 | 3.62 |
| 93 | 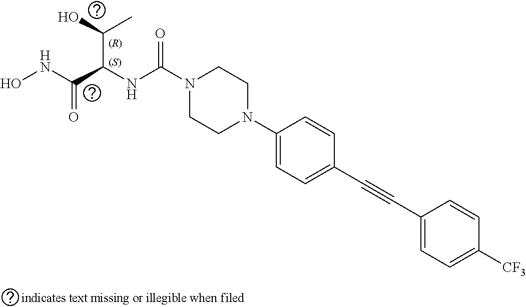 | 490.2 | 491.2 | 4.31 |
| 94 | 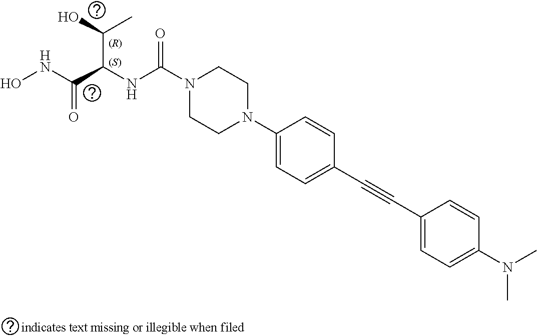 | 465.2 | 466.2 | 3.01 |
| 95 | 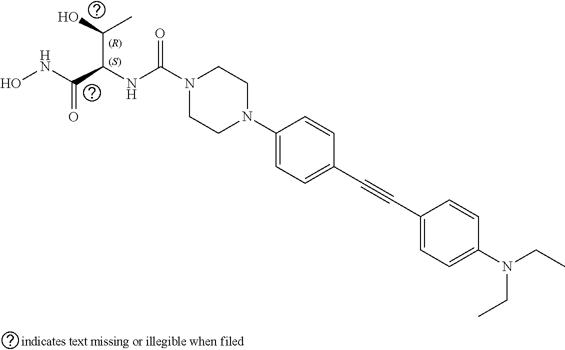 | 493.3 | 494.2 | 2.75 |
| 96 | 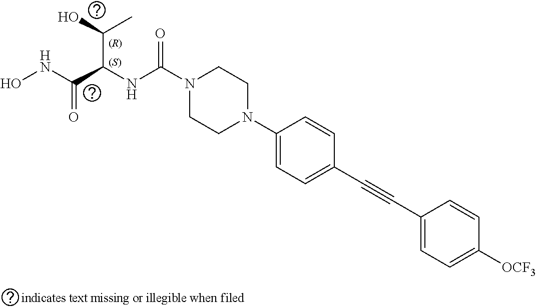 | 506.2 | 507.2 | 4.37 |
| 97 | 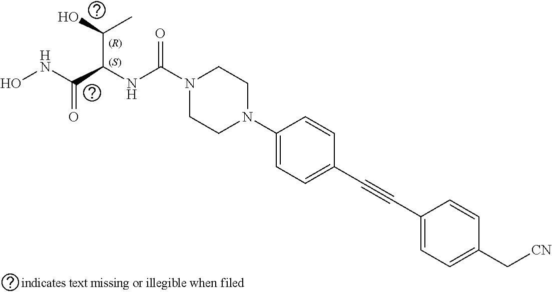 | 461.2 | 462.2 | 3.29 |

### Example 6

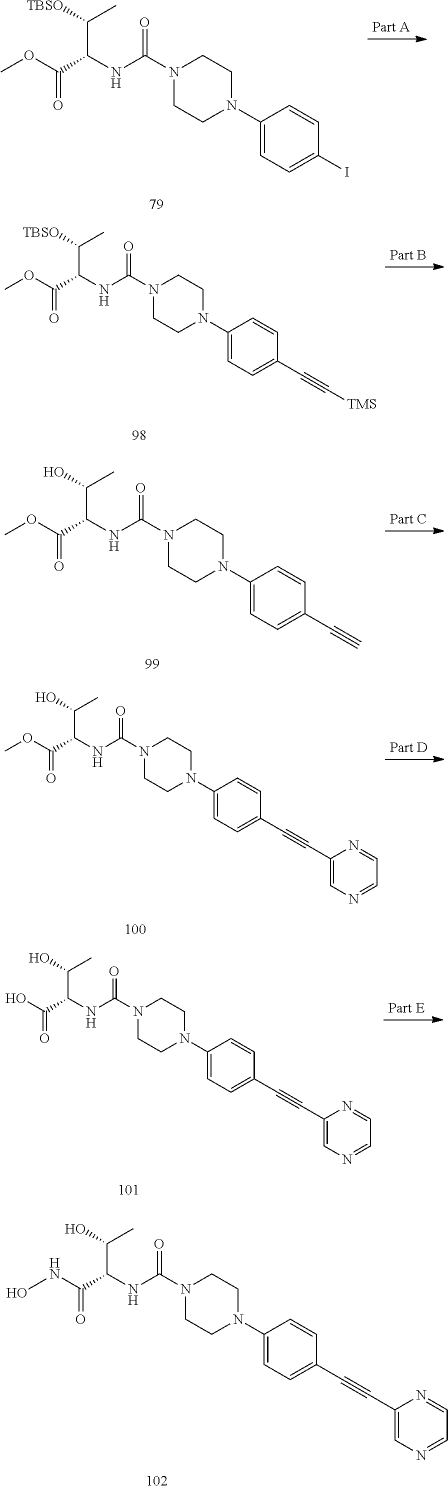

Part A:

Compound 98 (345 mg, 66%) was prepared from the reaction of compound 29 (550 mg, 0.98 mmol) with trimethylsilylacetylene (177 μL, 1.27 mmol) using the Sonagashira coupling conditions described in Example 5, Part H. HPLC-MS t_{R}=3.05 min (UV_{254 nm}); mass calculated for formula C₂₇H₄₅N₃O₄Si 531.3, observed LCMS m/z 532.3 (M+H).

Part B:

A solution containing compound 98 (345 mg, 0.65 mmol) and tetrabutylammonium fluoride (1M, 1.36 mL, 1.36 mmol) in THF (10 mL) was stirred at room temperature for 1 hour. LC-MS analysis indicated that the hydrolysis was complete. The reaction mixture was quenched with the addition of saturated NH₄Cl and extracted with EtOAc (2×10 mL). Drying over magnesium sulfate and concentration afforded crude compound 99 which was subjected to flash silica chromatography, gradient elution (0 to 100%) hexane/ethyl acetate (218 mg, 97%). HPLC-MS t_{R}=1.63 min (UV_{254 nm}); mass calculated for formula C₁₈H₂₃N₃O₄ 345.2, observed LCMS m/z 346.3 (M+H).

Part C:

Compound 100 (15 mg, 41%) was prepared from the reaction of compound 99 (30 mg, 0.09 mmol) with iodopyrazine (13 μL, 0.13 mmol) using the Sonagashira coupling conditions described in Example 5, Part H. HPLC-MS t_{R}=1.35 min (UV_{254 nm}); mass calculated for formula C₂₂H₂₅N₅O₄ 423.2, observed LCMS m/z 424.1 (M+H).

Part D:

Compound 101 (14 mg, 100%) was prepared from compound 100 (15 mg, 0.035 mmol) using the saponification conditions described in Example 5, Part F. HPLC-MS t_{R}=1.40 min (UV_{254 nm}); mass calculated for formula C₂₁H₂₃N₅O₄ 409.2, observed LCMS m/z 410.1 (M+H).

Part E:

Compound 102 (6.0 mg, 43%) was prepared from compound 101 (14 mg, 0.035 mmol) using the peptide coupling conditions described in Example 2, Part D. Purification by Prep.HPLC afforded compound 102 as an off white solid.

The compounds 102-114 (Table-5) were synthesized using the procedure described in example 6:

TABLE-5

| | | | | Ret. |
|---|---|---|---|---|
| Compound | | Exact | MS m/z | Time |
| Number | Structure | mass | (M⁺ + H) | (min) |
| 102 | 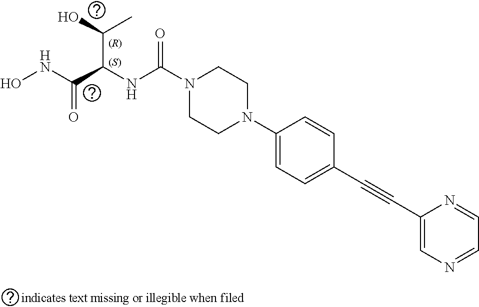 | 424.2 | 425.1 | 2.63 |
| 103 | 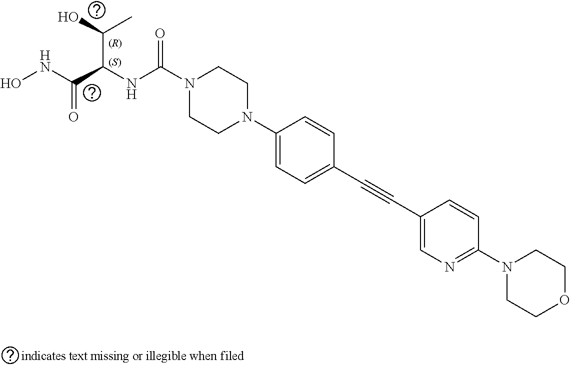 | 508.2 | 509.2 | 2.57 |
| 104 | 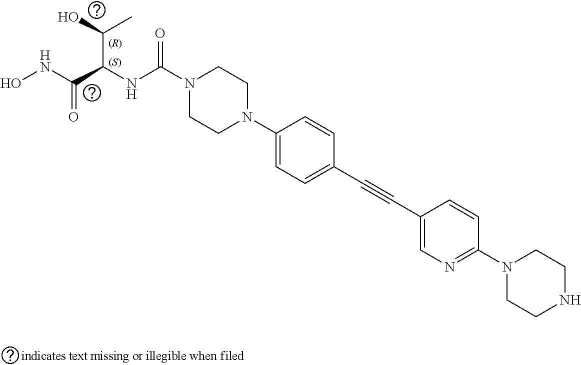 | 507.3 | 508.2 | 2.24 |
| 105 | 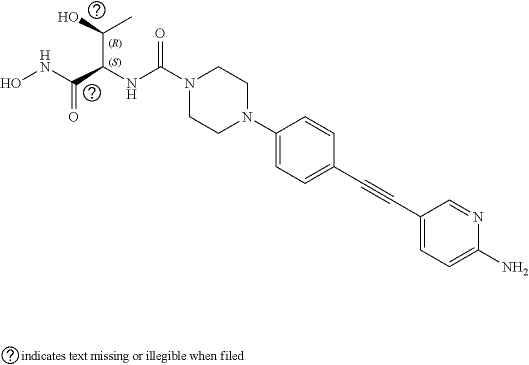 | 438.2 | 439.2 | 1.99 |
| 106 | 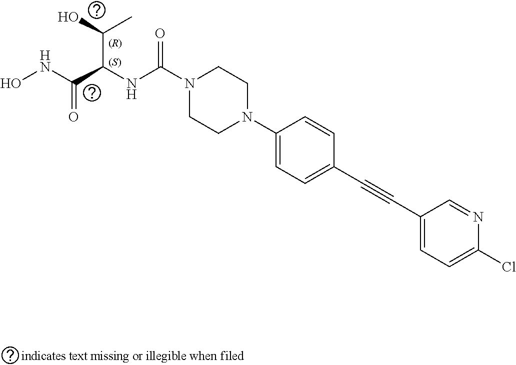 | 457.2 | 458.1 | 3.47 |
| 107 | 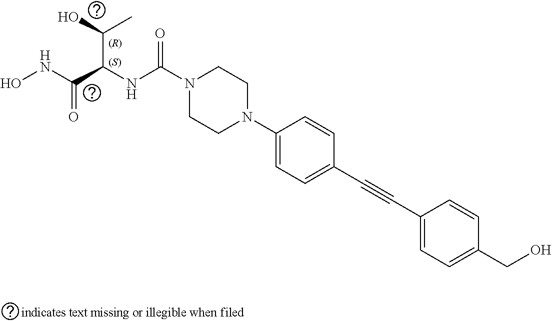 | 452.2 | 453.2 | 2.93 |
| 108 | 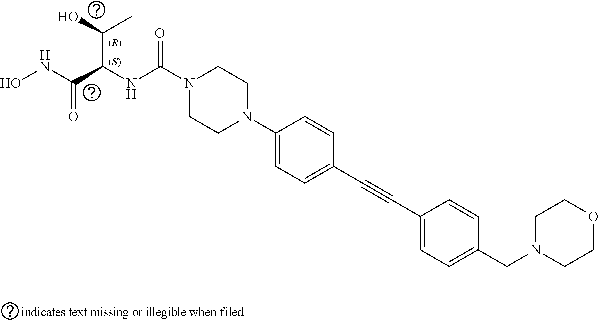 | 521.3 | 522.3 | 2.40 |
| 109 | 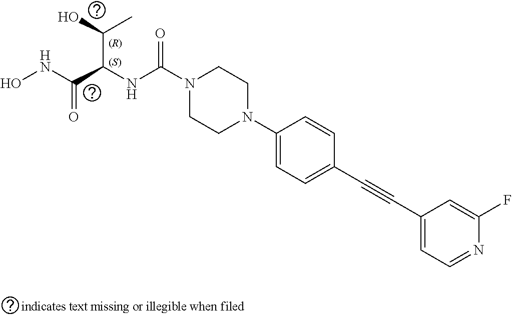 | 441.2 | 442.2 | 3.22 |
| 110 | 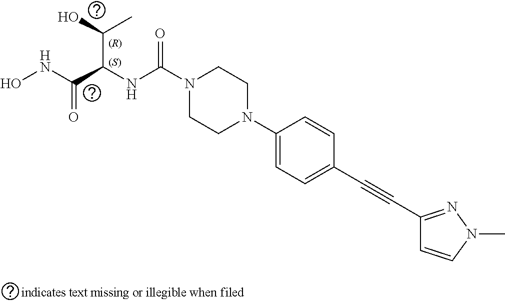 | 426.2 | 427.2 | 2.60 |
| 111 | 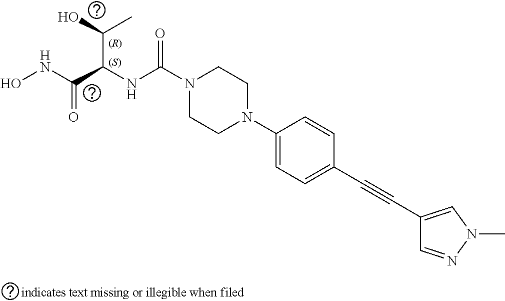 | 426.2 | 427.2 | 2.59 |
| 112 | 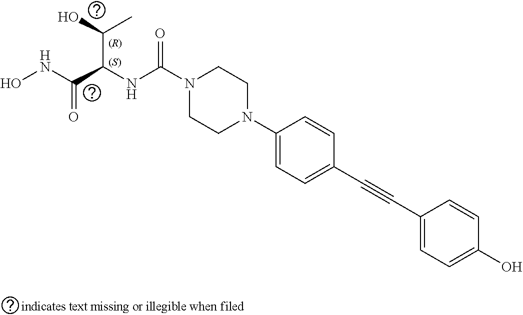 | 438.2 | 439.1 | 2.83 |
| 113 | 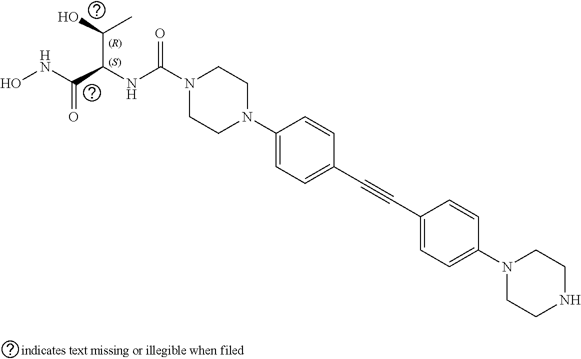 | 506.3 | 507.2 | 2.45 |
| 114 | 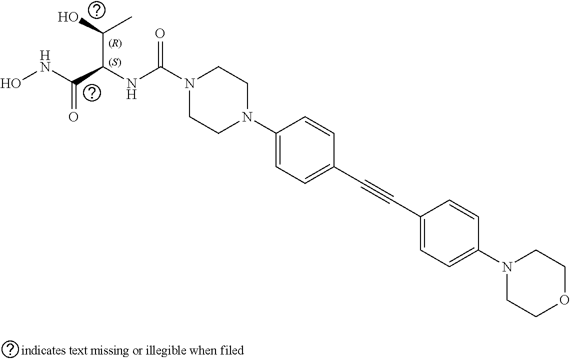 | 507.2 | 508.2 | 3.45 |

### Example 7

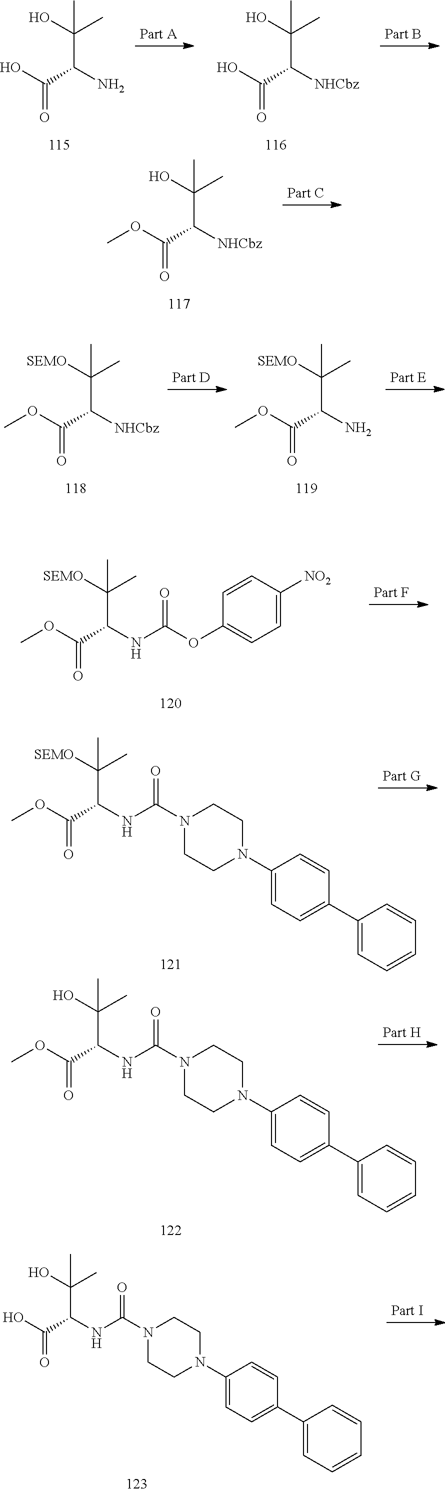

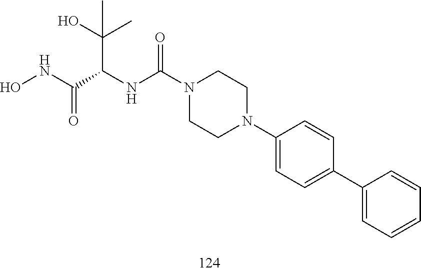

Part A:

To a solution of (S)-(+)-2-amino-3-hydroxy-3-methylbutanoic acid (115) (1.05 g, 7.90 mmol) in 2M NaOH (8 mL) at 0° C. was added a solution benzylchloroformate (1.11 mL, 7.90 mmol) in dioxane (13 mL). The reaction mixture was warmed to room temperature and stirred for 18 hours. The reaction mixture was acidified to pH 4.0 with 1N HCl and extracted with EtOAc (3×100 mL). Drying over magnesium sulfate and concentration afforded crude compound 116 as a colorless oil (1.81 g, 86%). HPLC-MS t_{R}=1.20 min (UV_{254 nm}); mass calculated for formula C₁₃H₁₇NO₅ 267.1, observed LCMS m/z 268.1 (M+H).

Part B:

To a solution of compound 116 (956 mg, 3.58 mmol) in MeCN (10 mL) and MeOH (10 mL) was added trimethylsilydiazomethane (1.8 mL, 3.58 mmol). The reaction mixture was stirred at room temperature for 20 minutes and concentrated to afford crude compound 117 as a yellow oil (1.05 g, 100%). HPLC-MS t_{R}=1.43 min (UV_{254 nm}); mass calculated for formula C₁₄H₁₉NO₅ 281.1, observed LCMS m/z 282.1 (M+H).

Part C:

To a solution of compound 117 (500 mg, 1.78 mmol) in DCM (5 mL) at 0° C. was added DIEA (372 μL, 1.78 mmol) and 2-(chloromethoxy)ethyltrimethylsilane (312 μL, 1.78 mmol). The reaction mixture was warmed to room temperature and then heated at 60° C. for 3.5 hours. Concentration and purification by flash silica chromatography, gradient elution (0 to 100%) hexane/ethyl acetate afforded compound 118 as a colorless oil (383 mg, 52%). HPLC-MS t_{R}=2.48 min (UV_{254 nm}); mass calculated for formula C₂₀H₃₃NO₆Si 411.2, observed LCMS m/z 434.1 (M+Na).

Part D:

Compound 119 (200 mg, 83%) was prepared from compound 118 using the hydrogenation conditions described in Example 5, Part C. HPLC-MS t_{R}=1.24 min (UV_{254 nm}); mass calculated for formula C₁₂H₂₇NO₄Si 277.2, observed LCMS m/z 278.2 (M+H).

Part E

Compound 120 (167 mg, 58%) was prepared from compound 119 using the conditions described in Example 2, Part A.

Part F:

Compound 121 (40 mg, 67%) was prepared from the reaction of compound 120 (50 mg, 1.12 mmol) with 1-biphenyl-4-yl-piperazine using the conditions described in Example 2, Part B. HPLC-MS t_{R}=2.57 min (UV_{254 nm}); mass calculated for formula C₂₉R₄₃N₃O₅Si 541.3, observed LCMS m/z 542.2 (M+H).

Part G:

A mixture of compound 121 (40 mg, 0.074 mmol) and 4N HCl in dioxane (2 mL) was heated at 50° C. for 2 hours. The reaction mixture was concentrated to afford crude compound 122 as a white solid (30 mg, 100%). HPLC-MS t_{R}=1.93 min (UV_{254 nm}); mass calculated for formula C₂₃H₂₉N₃O₄ 411.3, observed LCMS m/z 412.3 (M+H).

Part H:

Compound 123 (34 mg, 100%) was prepared from compound 122 using the saponification conditions described in Example 5, Part F. HPLC-MS t_{R}=1.93 min (UV_{254 nm}); mass calculated for formula C₂₂H₂₇N₃O₄397.2, observed LCMS m/z 398.3 (M+H).

Part I:

Compound 124 (6 mg, 19%) was prepared from compound 123 (34 mg, 0.078 mmol) using the peptide coupling conditions described in Example 2, Part D. Purification by Prep.HPLC afforded compound 124 as an off white solid.

The following compounds 124-127 (Table-6) were synthesized as described in example 7:

TABLE-6

| | | | | Ret. |
|---|---|---|---|---|
| Compound | | Exact | MS m/z | Time |
| Number | Structure | mass | (M⁺ + H) | (min) |
| 124 | 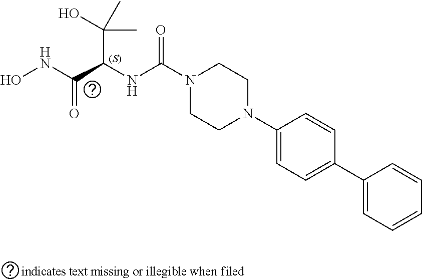 | 412.2 | 413.2 | 3.14 |
| 125 | 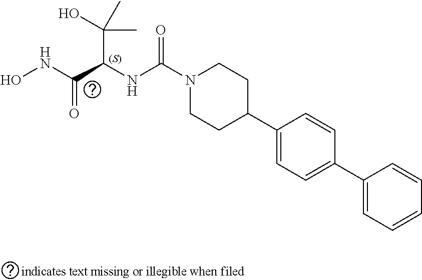 | 411.2 | 412.2 | 3.69 |
| 126 | 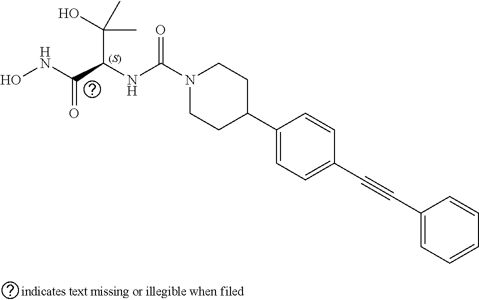 | 436.2 | 437.2 | 3.80 |
| 127 | 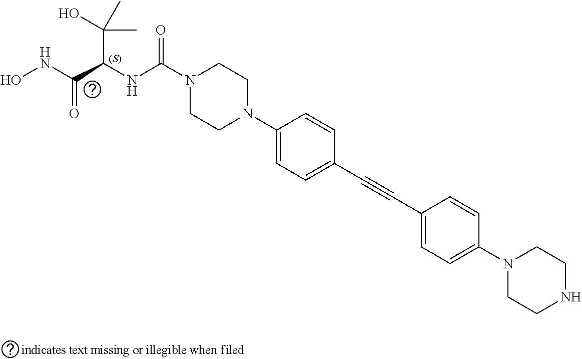 | 520.3 | 521.3 | 1.00 |

### Example 8

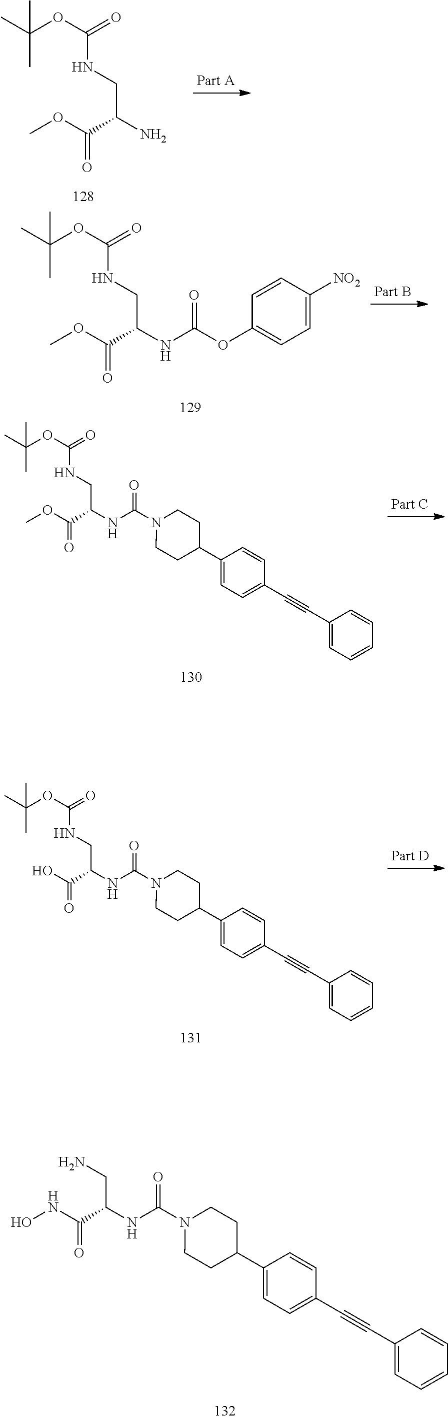

Part A:

Compound 129 (101 mg, 23%) was prepared from compound 128 using the conditions described in Example 2, Part A.

Part B:

Compound 130 (21 mg, 81%) was prepared from the reaction of compound 129 (20 mg, 0.052 mmol) with compound 3 using the conditions described in Example 2, Part B. HPLC-MS t_{R}=2.23 min (UV_{254 nm}); mass calculated for formula C₂₉H₃₅N₃O₅ 505.3, observed LCMS m/z 506.3 (M+H).

Part C:

Compound 131 (20 mg, 100 was prepared from compound 130 using the saponification conditions described in Example 5, Part F. HPLC-MS t_{R}=2.10 min (UV_{254 nm}); mass calculated for formula C₂₈H₃₃N₃O₅ 491.2, observed LCMS m/z 492.2 (M+H).

Part D:

Compound 132 (5 mg, 24%) was prepared from compound 131 (20 mg, 0.042 mmol) using the peptide coupling conditions described in Example 2, Part D. The BOC-protection group was hydrolyzed by stirring with trifluoroacetic acid (2 mL) for 1 minute at room temperature.

The volatiles were removed in vacuo and the crude residue submitted for purification by Prep.HPLC to afford compound 132 as an off white solid.

The following compound, 132, (Table-7) was synthesized using this procedure described in example 8:

TABLE-7

| | | | | Ret. |
|---|---|---|---|---|
| Compound | | Exact | MS m/z | Time |
| Number | Structure | mass | (M⁺ + H) | (min) |
| 132 | 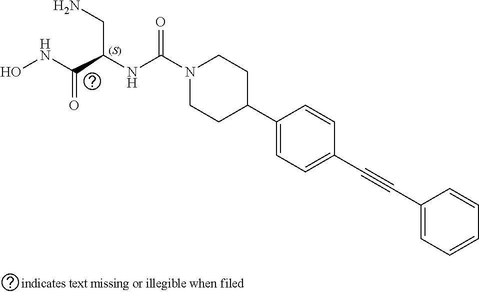 | 506.3 | 507.2 | 3.87 |

### Example 9

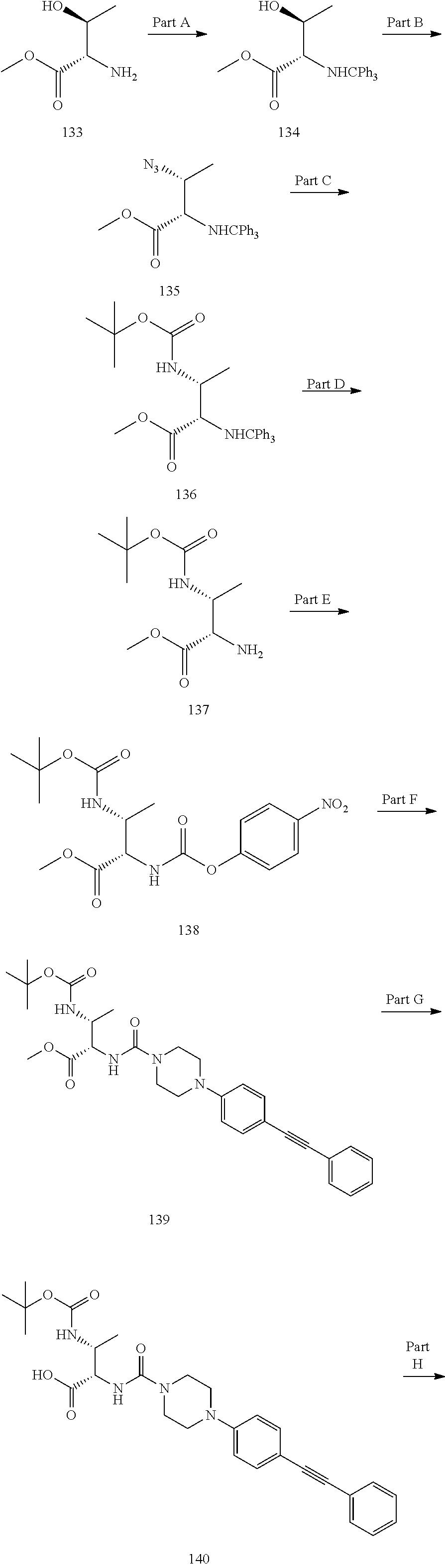

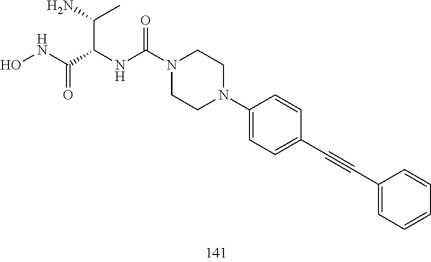

Part A:

A solution of tritylbromide (1.59 g, 4.92 mmol) in chloroform (20 mL) was slowly added at room temperature to a stirred mixture of H-allo-threonine methyl ester hydrochloride (1.0 g, 5.89 mmol) and DIEA (2.57 mL, 17.67 mmol) in chloroform (30 mL). The reaction mixture was stirred for 18 hours. LC-MS analysis confirmed the reaction was complete. The volatiles were removed in vacuo, the residue re-dissolved in EtOAc and washed with 0.1N HCl. Drying over magnesium sulfate and concentration afforded crude compound 134 which was subjected to flash silica chromatography, gradient elution (0 to 100%) hexane/ethyl acetate (1.54 g, 70%). HPLC-MS t_{R}=2.11 min (UV_{254 nm}); mass calculated for formula C₂₄H₂₅NO₃ 375.2, observed LC-MS m/z 378.2 (M+Na).

Part B:

To an ice-cooled solution of compound 134 (1.54 g, 4.1 mmol) and triphenylphosphine (1.08 g, 4.1 mmol) in THF (15 mL) was added diethyl azodicarboxylate (1.08 mL, 6.6 mmol) in THF (3 mL) under an argon atmosphere. The reaction mixture was stirred at 0° C. for 10 minutes. A solution of diphenylphosphoryl azide (2.4 mL, 11.1 mmol) in THF (2 mL) was added and the reaction mixture warmed to room temperature and stirred for an additional 18 hours. The volatiles were removed in vacuo, and the resulting residue subjected to flash silica chromatography, gradient elution (0 to 100%) hexane/ethyl acetate to afford compound 135 as a yellow oil (1.30 g, 80%).

Part C:

A mixture of compound 135 (518 mg, 1.3 mmol), triphenylphosphine (680 mg, 2.6 mmol) and water (100 μL) in THF (5 mL) was heated at 60° C. for 18 hours. The reaction mixture was cooled to room temperature and quenched with the addition of saturated NaHCO₃. Extraction with EtOAc (2×10 mL), drying over magnesium sulfate and concentration afforded crude compound 136 as the free amine. This residue was re-dissolved in DCM (5 mL), DIEA (677 μL, 3.9 mmol) and di-tert-butylcarbonate (339 mg, 1.56 mmol) added, and the reaction mixture stirred at room temperature for 1 hour. LC-MS analysis confirmed the reaction was complete. The reaction mixture was washed with 0.1N HCl, dried over magnesium sulfate and concentrated to afford erode compound 136 which was subjected to flash silica chromatography, gradient elution (0 to 100%) hexane/ethyl acetate (450 mg, 73%). HPLC-MS t_{R}=2.55 min (UV_{254 nm}); mass calculated for formula C₂₉H₃₄N₂O₄ 474.3, observed LCMS m/z 497.2 (M+Na).

Part D:

A solution of compound 136 (507 mg, 1.07 mmol) and palladium hydroxide on charcoal (20%) in a mixture of EtOAc (10 mL) and Mean (10 mL) was subjected to hydrogenation for 18 hours at 45 p.s.i. LC-MS analysis indicated the reaction was complete. The reaction mixture was filtered by passing through celite, and evaporated to afford crude compound 137 as a colorless oil (248 mg, 100%). HPLC-MS t_{R}=0.85 min (UV_{254 nm}); mass calculated for formula C₁₀H₂₀N₂O₄ 232.1, observed LCMS m/z 233.1 (M+H).

Part E:

Compound 138 (101 mg, 20%) was prepared from compound 137 using the conditions described in Example 2, Part A. HPLC-MS t_{R}=1.81 min (UV_{254 nm}); mass calculated for formula C₁₇H₂₃N₃O₈ 397.1, observed LCMS m/z 398.1 (M+H).

Part F:

Compound 139 (20 mg, 38%) was prepared from the reaction of compound 139 (40 mg, 0.1 mmol) with compound 6 using the conditions described in Example 2, Part B. HPLC-MS t_{R}=2.26 min (UV_{254 nm}); mass calculated for formula C₂₉H₃₆N₄O₅ 520.3, observed LCMS m/z 521.3 (M+H).

Part G:

Compound 140 (19 mg, 100%) was prepared from compound 139 using the saponification conditions described in Example 5, Part F. HPLC-MS t_{R}=2.10 min (UV_{254 nm}); mass calculated for formula C₂₈H₃₄N₄O₅ 506.3, observed LCMS m/z 507.3 (M+H).

Part H:

Compound 141 (9.1 mg, 57%) was prepared from compound 140 (19 mg, 0.038 mmol) using the peptide coupling conditions described in Example 2, Part D. The BOC-protecting group was hydrolyzed by stirring with trifluoroacetic acid (2 mL) for 1 minute at room temperature. The volatiles were removed in vacuo and the crude residue submitted for purification by Prep.HPLC to afford compound 141 as an off white solid.

The compounds 141-144 (Table-8) were synthesized using the procedure described in the example 9:

TABLE-8

| | | | | Ret. |
|---|---|---|---|---|
| Compound | | Exact | MS m/z | Time |
| Number | Structure | mass | (M⁺ + H) | (min) |
| 141 | 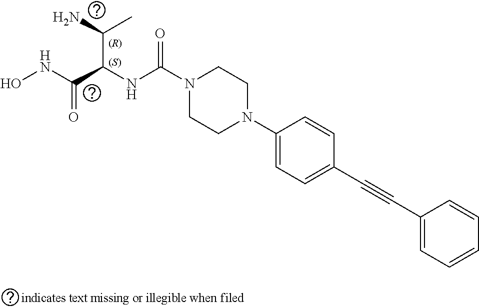 | 421.2 | 422.2 | 3.29 |
| 142 | 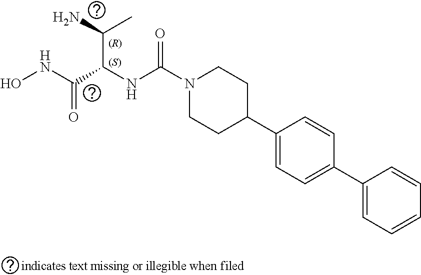 | 396.2 | 397.2 | 3.14 |
| 143 | 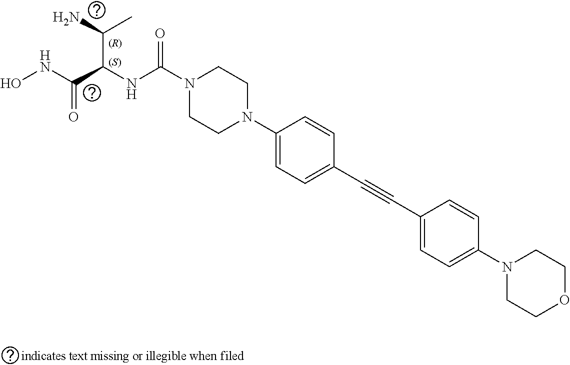 | 506.3 | 507.2 | 3.11 |
| 144 | 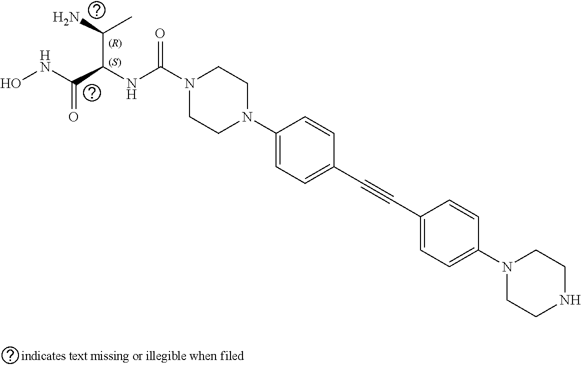 | 505.3 | 506.3 | 2.15 |

### Example 10

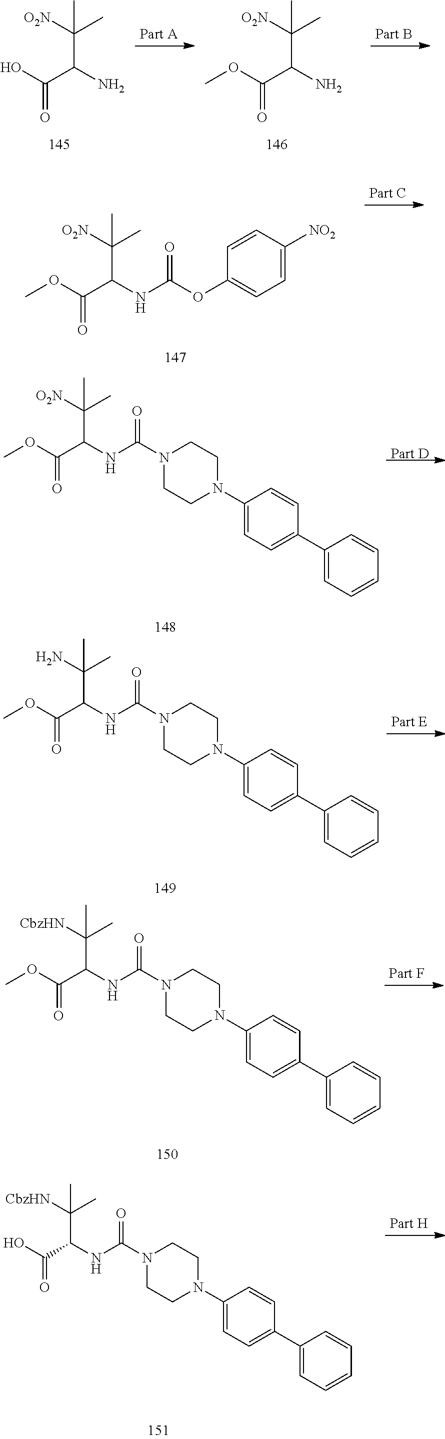

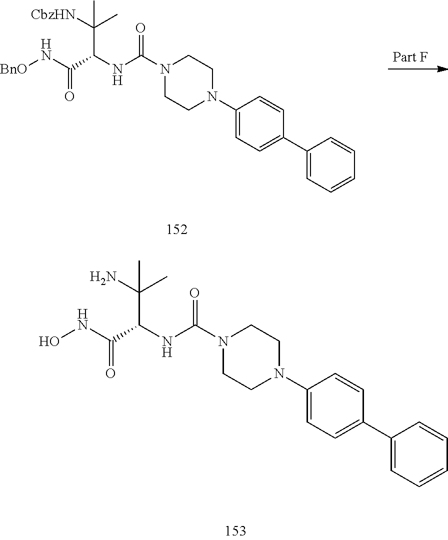

Compound 145 was synthesized according to reference J. Chem. Perkin. Trans. 1, (1999), 2659.

Part A:

To a solution of compound 145 (1.1 g, 5.54 mmol) in MeOH (10 mL) was added concentrated H₂SO₄ (4 mL). The reaction mixture was refluxed for 18 hours, then cooled to room temperature and concentrated. The resulting residue was diluted with water, basified with saturated NaHCO₃ and extracted with EtOAc. Drying over magnesium sulfate and concentration afforded crude compound 146 as a colorless oil (800 mg, 82%). HPLC-MS t_{R}=1.0 min (UV_{254 nm}); mass calculated for formula C₆H₁₂N₂O₄ 176.1, observed LCMS m/z 177.1 (M+H).

Part B:

Compound 147 (500 mg, 20%) was prepared from compound 146 using the conditions described in Example 2, Part A.

Part C:

Compound 148 (85 mg, 14%) was prepared from the reaction compound 147 (450 mg, 1.32 mmol) with 1-biphenyl-4-yl-piperazine using the conditions described in Example 2, Part B.

HPLC-MS t_{R}=2.25 min (UV_{254 nm}); mass calculated for formula C₂₃H₂₈N₄O₅ 440.2, observed LCMS m/z 442.2 (M+2H).

Part D:

A mixture of compound 148 (30 mg, 0.068 mmol) and Raney nickel in EtOH (10 mL) was subjected to hydrogenation at 1 a.t.m. for 18 hours. The reaction mixture was filtered by passing through celite, concentrated and purified by prep.HPLC to afford compound 149 as a white solid (5 mg, 18%). HPLC-MS t_{R}=3.41 min (UV_{254 nm}); mass calculated for formula C₂₃H₃₀N₄O₃ 410.2, observed LCMS m/z 412.2 (M+2H).

Part E:

To an ice-cooled solution of compound 149 (30 mg, 0.073 mmol) and DIEA (38 μL, 0.219 mmol) in DCM (3 mL) was added benzyl chloroformate (10 μL, 0.177 mmol). The reaction mixture was heated at 60° C. for 18 hours, cooled to room temperature, diluted with DCM and washed with 1N HCl. Drying over magnesium sulfate and concentration afforded crude compound 150 as a colorless oil (35 mg, 88%). HPLC-MS t_{R}=2.32 min (UV_{254 nm}); mass calculated for formula C₃₁H₃₆N₄O₅ 544.3. observed LCMS m/z 546.2 (M+2H).

Part F:

Compound 151 (33 mg, 100%) was prepared from compound 150 using the saponification conditions described in Example 5, Part F. HPLC-MS t_{R}=2.14 min (UV_{254 nm}); mass calculated for formula C₃₀H₃₄N₄O₅ 530.3, observed LCMS m/z 532.3 (M+2H).

Part G:

Compound 152 (20 mg, 25%) was prepared from the reaction of compound 151 (67 mg, 0.126 mmol) with O-benzylhydroxylamine using the peptide coupling conditions described in Example 2, Part D. HPLC-MS t_{R}=2.31 min (UV_{254 nm}); mass calculated for formula C₃₇H₄₁N₄O₅ 635.3, observed LCMS m/z 637.3 (M+2H).

Part H:

Compound 153 (19 mg, 15%) was prepared from compound 152 using the hydrogenation conditions described in Example 9, Part D. The crude residue was submitted for purification by Prep.HPLC to afford compound 151 as an off white solid.

The following compound, 151 (Table-9) was synthesized using the procedure described in example 10:

TABLE-9

| | | | | Ret. |
|---|---|---|---|---|
| Compound | | Exact | MS m/z | Time |
| Number | Structure | mass | (M⁺ + H) | (min) |
| 151 | 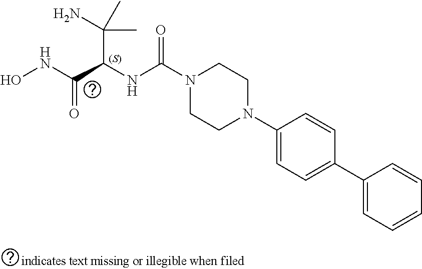 | 411.2 | 412.2 | 2.91 |

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

Each and every document referred to in this patent application is incorporated herein by reference in its entirety for all purposes.

1. A compound represented by Formula (I) 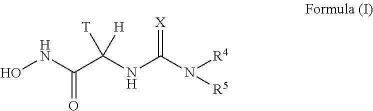
or a pharmaceutically acceptable salt, solvate, or ester thereof, wherein:
(i) T is selected from the group consisting of H, alkyl, alkenyl and alkynyl, wherein said alkyl, alkenyl and alkynyl can be unsubstituted or optionally independently substituted with one or more moieties selected from the group consisting of aryl, aralkyl, heteroaryl, heteroaralkyl, cycloalkyl, cycloalkylalkyl, cycloalkenyl, cycloalkenylalkyl, heterocyclyl, heterocyclenyl, heterocycloalkylalkyl, heterocyclenylalkyl, —OH, alkoxyl, —O-alkenyl, —O-alkynyl, hydroxyalkyl, hydroxyalkenyl, —O-aryl, —O-aralkyl, —SH, —S-alkyl, —S-alkenyl, —S-alkynyl, —S-aryl, —S-aralkyl, —NR¹R², -alkyl-NR¹R², -alkenyl-NR¹R²,
wherein R¹ and R² are independently selected from the group consisting of H, alkyl, alkenyl, alkynyl, aryl, cycloalkyl, cycloalkenyl, aralkyl, cycloalkylalkyl, cycloalkenylalkyl, heteroaryl, heteroaralkyl, or
R¹ and R² together with the N atom to which each is attached form heterocyclyl, heterocyclenyl, or heteroaryl;or
T and H together with the C atom to which each is attached form spirocycloalkyl or spiroheterocyclyl, wherein each of said spirocycloalkyl and spiroheterocyclyl can be unsubstituted or optionally independently substituted with one or more moieties selected from the group consisting of alkyl, alkenyl, alkynyl, aryl, cycloalkenyl, cycloalkyl, aralkyl, aralkenyl, cycloalkenylalkyl, cycloalkylalkyl, halo and haloalkyl;
(ii) X is O, S or NH;
(iii) R⁴ and R⁵ are independently selected from the group consisting of hydrogen or (C₁-C₆)alkyl, wherein said (C₁-C₆)alkyl is substituted with aryl wherein said aryl can be unsubstituted or optionally independently substituted with alkynyl, halo, aryl, heteroaryl, heterocyclenyl or heterocyclyl, wherein said alkynyl, heteroaryl, heterocyclenyl or heterocyclyl can be unsubstituted or substituted with an additional aryl; or
R⁴ and R⁵ together with the N atom to which each is attached, form a heterocyclyl or heterocyclenyl, wherein each of said heterocyclyl and heterocyclenyl is substituted with A; or
R⁴ and R⁵ together with the N atom to which each is attached form a heterocyclic structure represented by the structure: 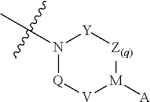
wherein Y, Q, Z, or V are each independently selected from the group consisting of C(O), C(S), C(NH), S(O), S(O)₂ and C(R⁶R⁷), wherein q is 0-1, wherein each of R⁶ and R⁷ are independently selected from the group consisting of H, alkyl, and alkenyl, wherein each of said alkyl or alkenyl can be unsubstituted or optionally independently substituted with one or more moieties selected from the group consisting of H, alkyl, alkenyl, alkynyl, aryl, cycloalkenyl, cycloalkyl, aralkyl, aralkenyl, cycloalkenylalkyl, cycloalkylalkyl, halo and haloalkyl;
further wherein M is N or CR, wherein R is H, halo, alkyl, alkenyl, alkynyl, aryl, aralkyl, cycloalkenyl, cycloalkyl, heteroaryl, heterocyclenyl, heterocyclyl, OH, —O-alkyl, —O-alkenyl, —O-alkynyl, —O-aryl, —O-aralkyl, —O-cycloalkenyl, —O-cycloalkyl, —O-heteroaryl, —O-heterocyclenyl, —O-heterocyclyl, —SH, —S-alkyl, —S-alkenyl, —S-alkynyl, —S-aryl, —S-aralkyl, —S-cycloalkenyl, —S-cycloalkyl, —S-heteroaryl, —S-heterocyclenyl, or —S-heterocyclyl;
A is selected from the group consisting of, -aryl-alkynyl-aryl, -aryl-C(O)aralkyl, -aryl, -biaryl, -alkynyl-aryl, -aryl-heteroaryl and -aryl-alkynyl-heteroaryl, wherein said , -aryl-alkynyl-aryl, -aryl-C(O)aralkyl, -aryl, -biaryl, -alkynyl-aryl, -aryl-heteroaryl, and -aryl-alkynyl-heteroaryl can be unsubstituted or optionally independently substituted with one or more moieties selected from the group consisting of halo, haloalkyl, —N(R¹)(R²), haloalkoxyl, -alkyl-CN, hydroxyalkyl, —OH, heterocyclyl, heterocyclenyl, alkyl, alkenyl, dialkylaminoalkoxyl and heterocyclylalkoxyl.

2. The compound of claim 1, wherein T is hydroxyalkyl.

3. The compound according to claim 1, wherein X is O.

4. The compound according to claim 1, wherein each of said R⁴ and R⁵ is independently hydrogen or (C₁-C₆)alkyl, wherein said (C₁-C₆)alkyl is substituted with aryl, wherein said aryl can be unsubstituted or optionally independently substituted with alkynyl, halo or heteroaryl, wherein said alkynyl is substituted with an additional aryl.

5. The compound according to claim 4, wherein said (C₁-C₆)alkyl can be straight chain alkyl or branched alkyl.

6. The compound according to claim 5, wherein said alkyl is methyl, ethyl or branched ethyl.

7. The compound according to claim 4, wherein said alkynyl is ethynyl.

8. The compound according to claim 4, wherein said halo is bromo.

9. The compound of claim 4, wherein said heteroaryl is N-pyrazole.

10. The compound according to claim 1, wherein said R⁴ and R⁵ together with the N atom to which each is attached form heterocyclyl, substituted with A.

11. The compound according to claim 10, wherein said heterocyclyl is piperazinyl, piperidinyl, pyrollidinyl.

12. The compound according to claim 10, wherein A is phenyl-ethynyl-phenyl, ethynyl-phenyl, phenyl-C(O)-benzyl, phenyl, biphenyl, phenyl-heteroaryl, phenyl-heteroaryl-heterocyclyl or phenyl-ethynyl-heteroaryl,
wherein said phenyl can be unsubstituted or optionally independently substituted with one or more moieties selected from the group consisting of chloro, bromo, —NH₂, dialkylamino, trihaloalkyl, —O-trihaloalkyl and cyanoalkyl;
wherein said biphenyl can be unsubstituted or optionally independently substituted with one or more moieties selected from the group consisting of propyl, fluro, heterocyclyl, dimethylaminoethoxyl and heterocyclylalkoxyl;
wherein said heteroaryl is selected from the group consisting of pyrimidinyl, pyridinyl, thiophenyl, thiazolyl, pyrazinyl and pyrazolyl, further wherein said heteroaryl can be unsubstituted or optionally independently substituted with one or more moieties selected from the group consisting of halo, alkyl, —NH₂ and heterocyclyl; and
wherein said heterocyclyl is selected from the group consisting of morpholinyl, piperazinyl, piperidinyl and pyrrolidinyl.

13. The compound according to claim 12, wherein said heteroaryl is selected from the group consisting of 5-pyrimidinyl, 4-pyridinyl, 3-thiophenyl, 5-thiazolyl, 2-pyrazinyl and 5-pyrazolyl.

14. The compound according to claim 12, wherein said heterocyclyl is selected from the group consisting of 4-morpholinyl, piperazinyl, piperidinyl and pyrrolidinyl.

15. The compound according to claim 12, wherein said biphenyl is substituted with 4-morpholinylethoxyl.

16. A compound selected from the group consisting of: 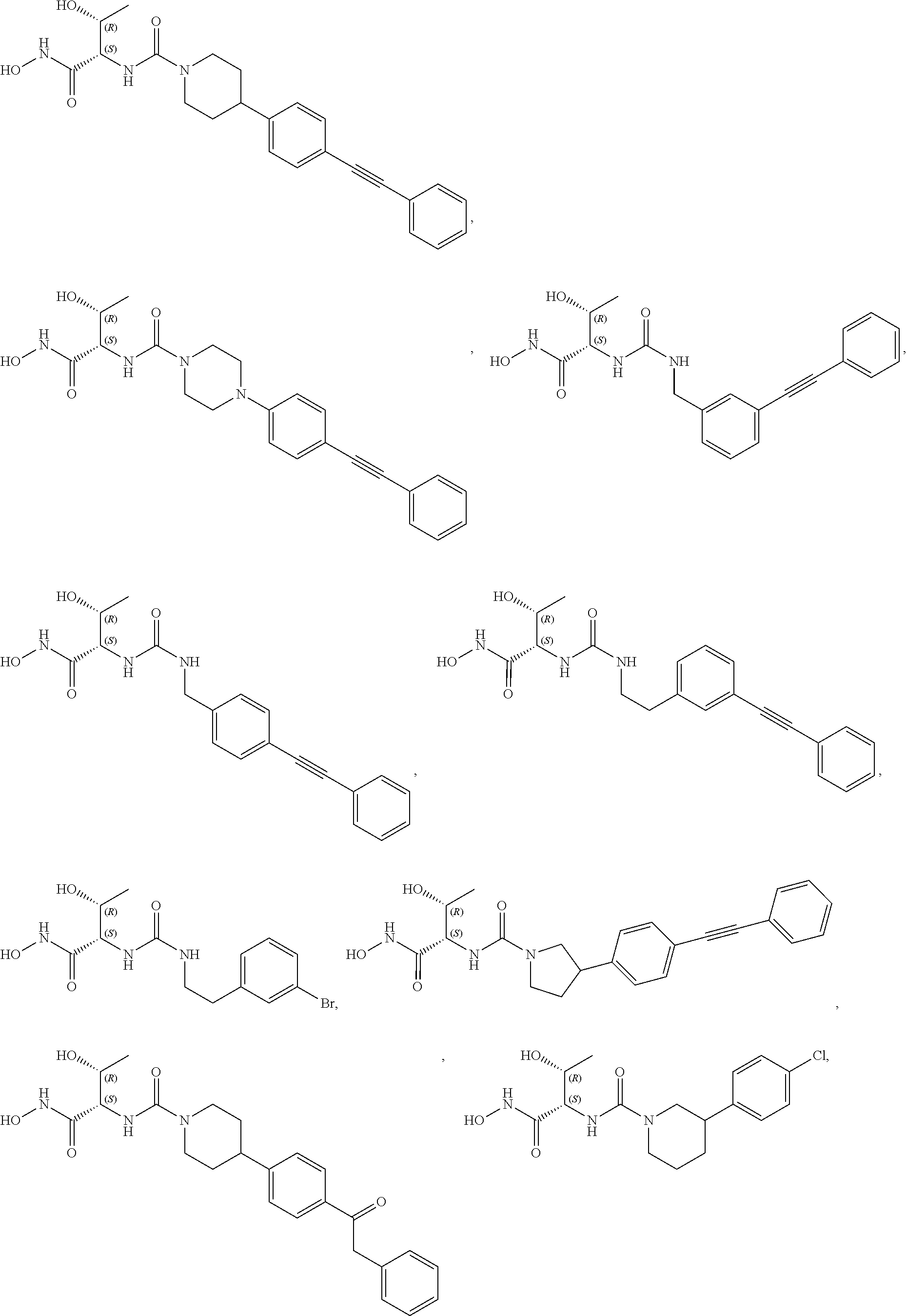 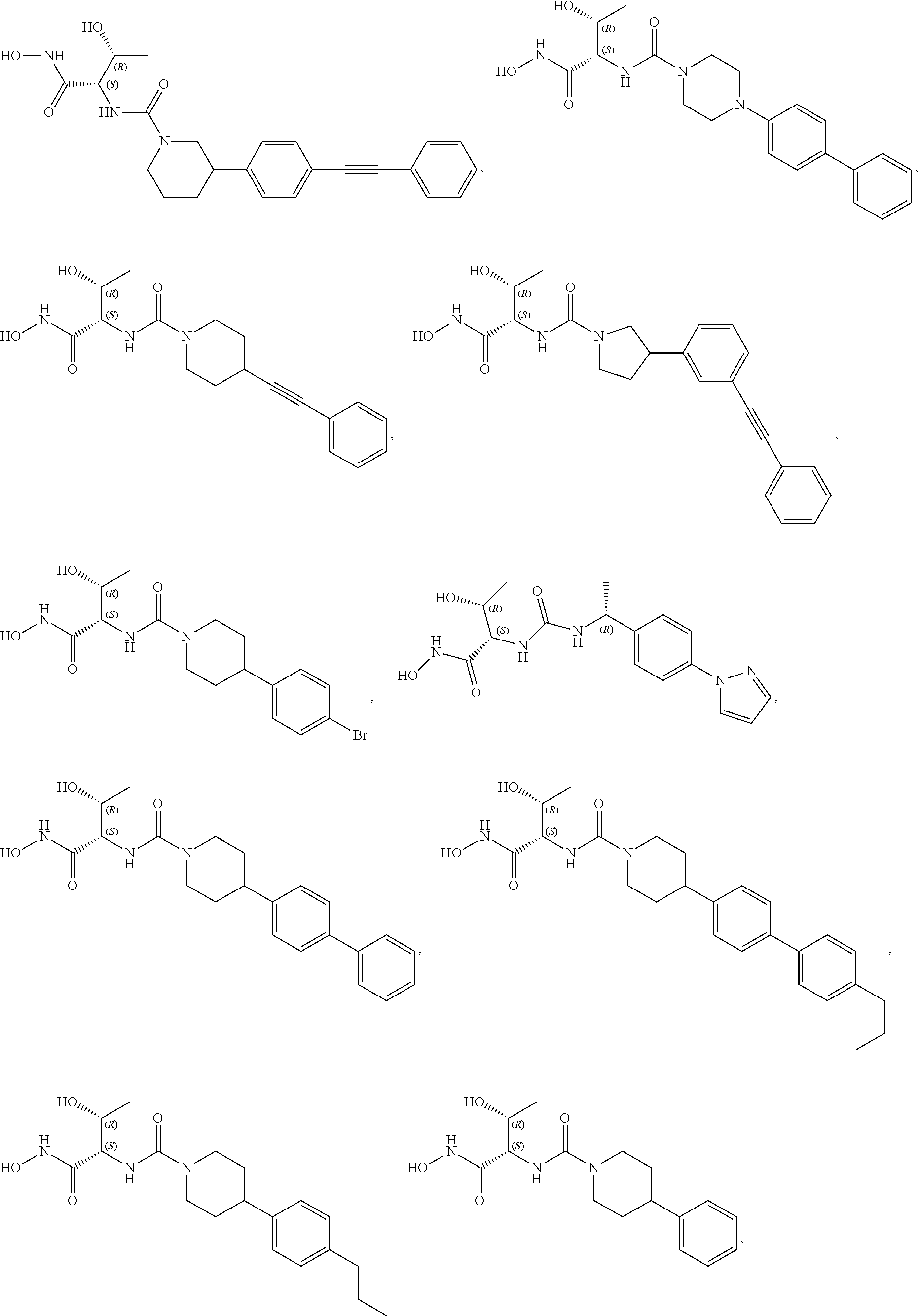 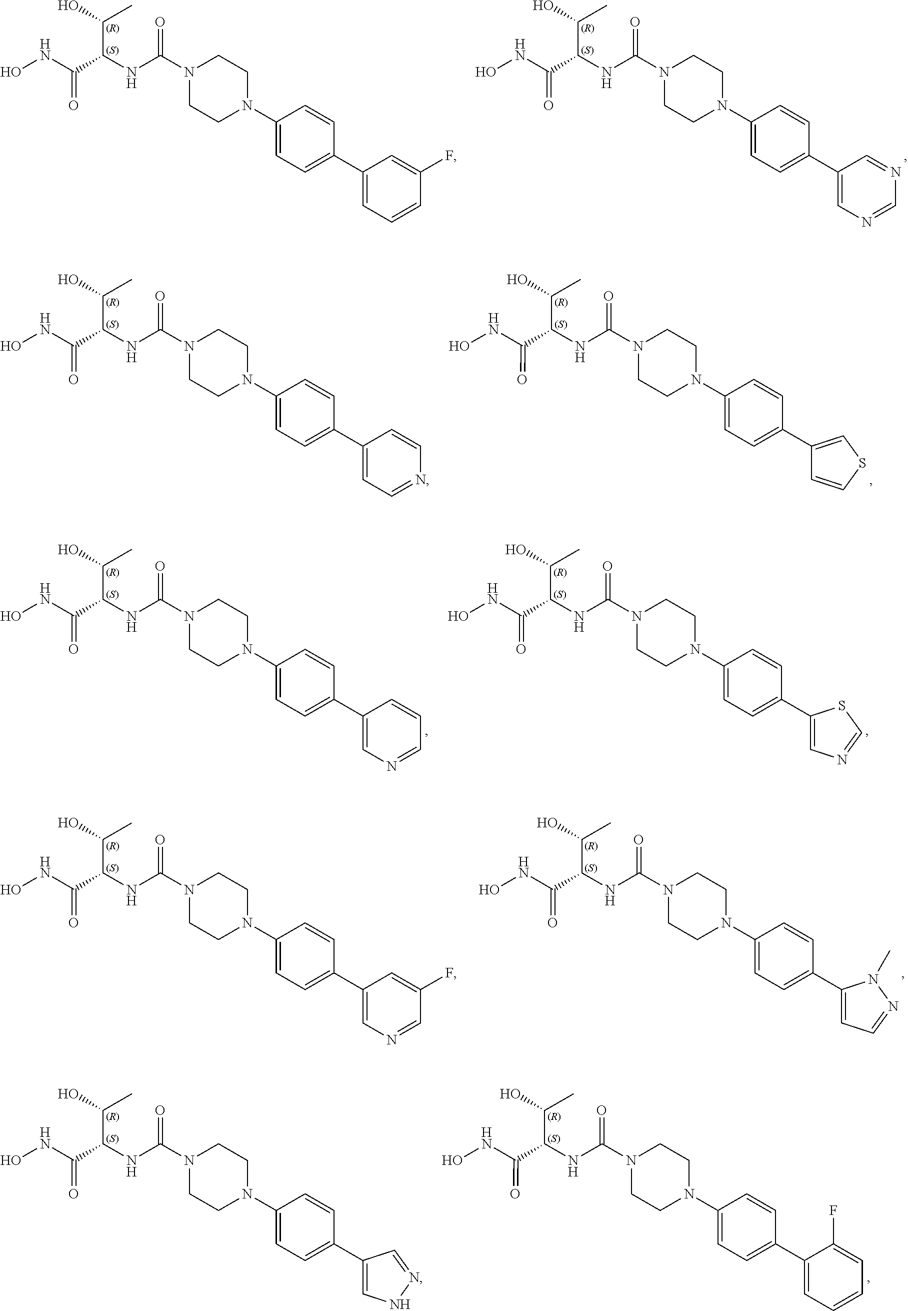 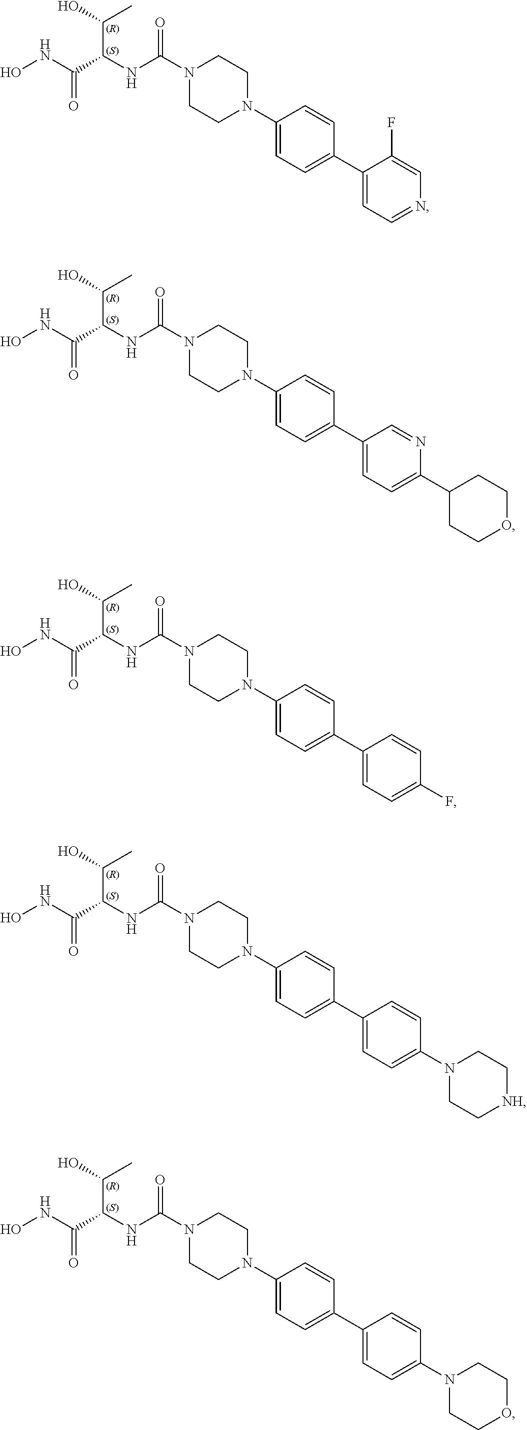 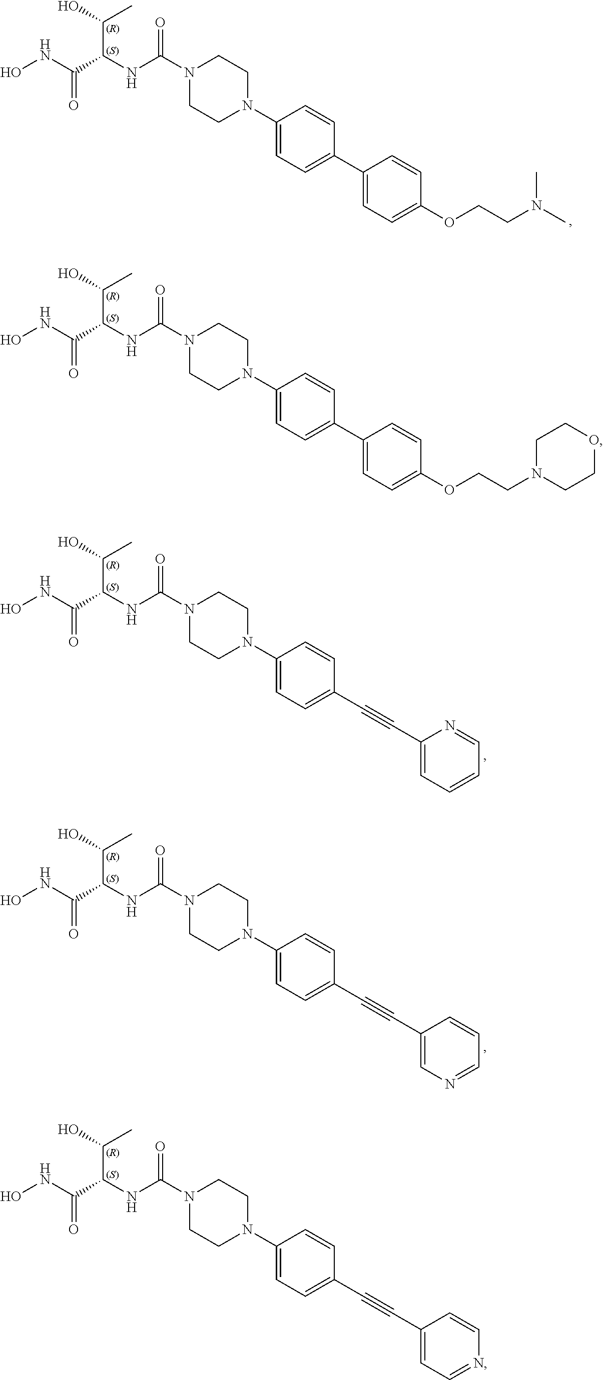 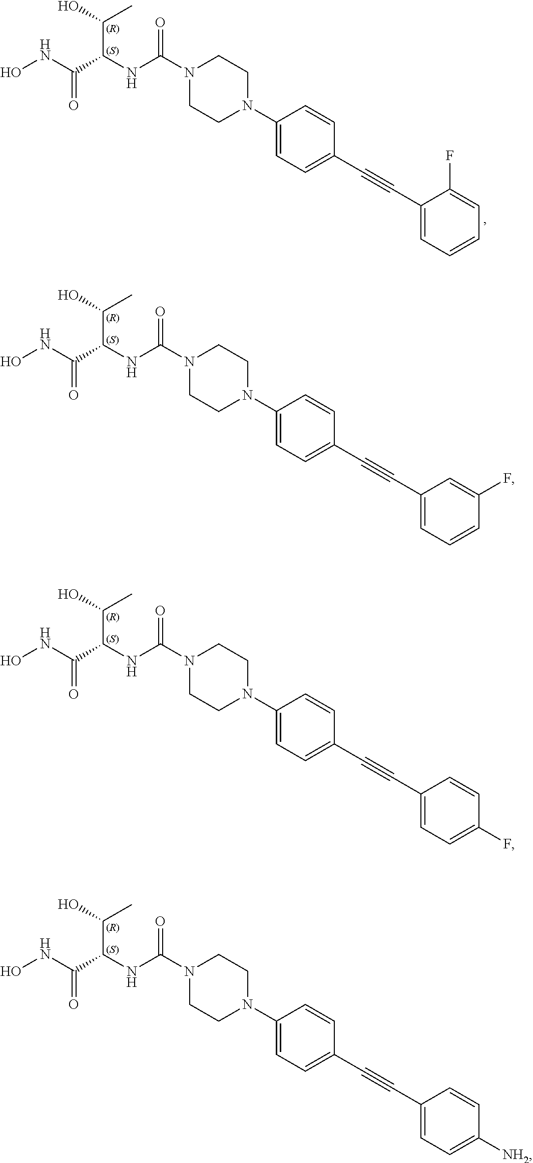 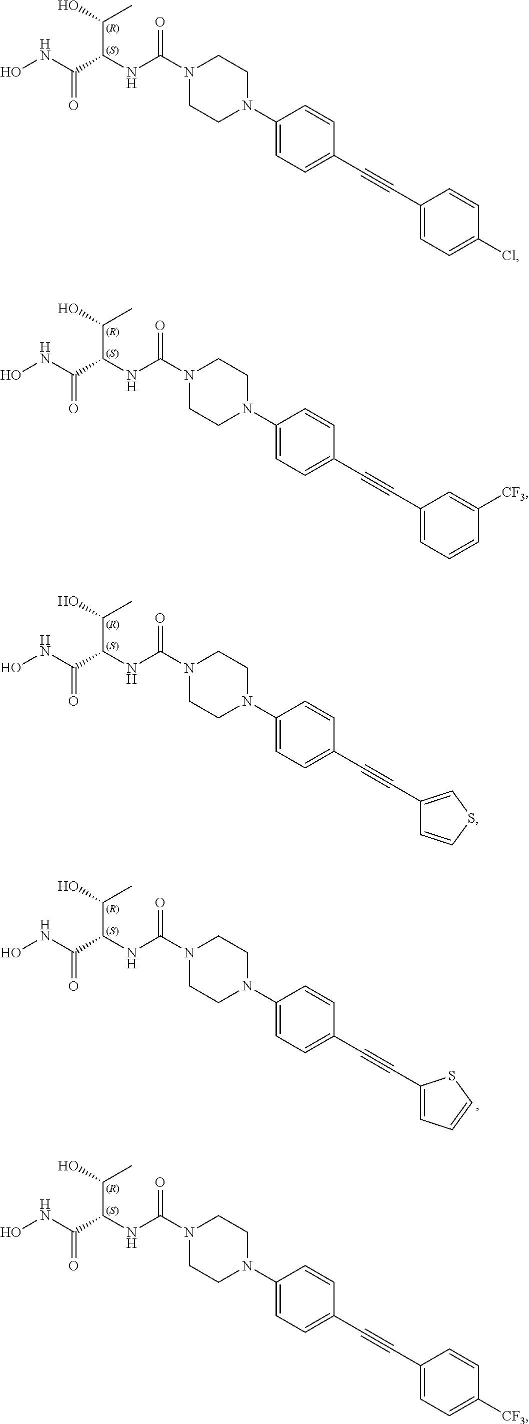 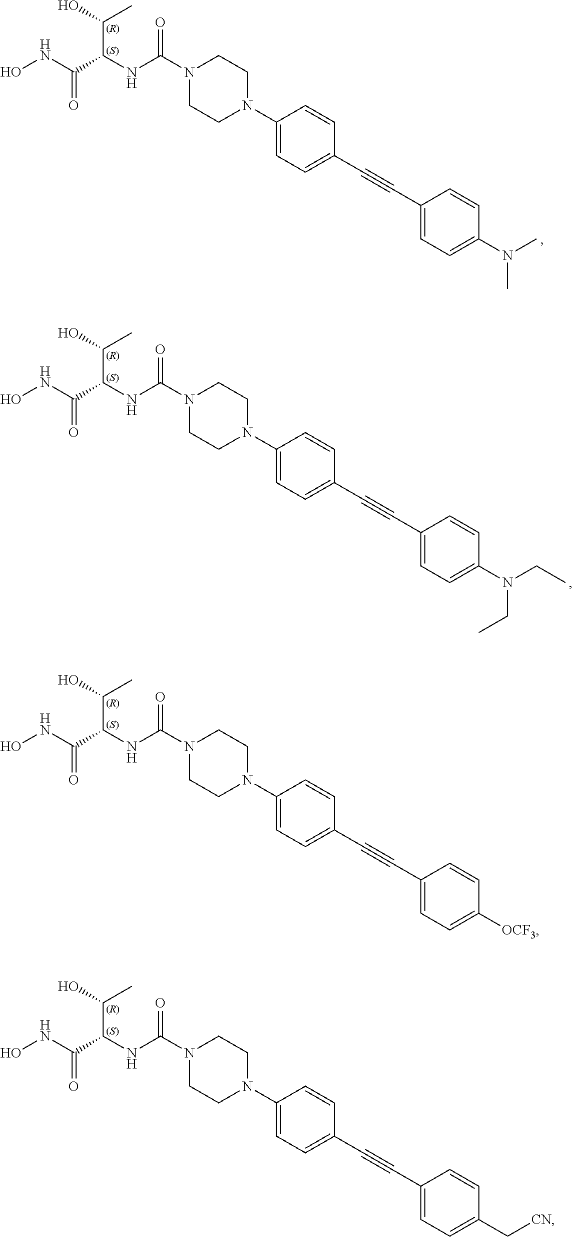 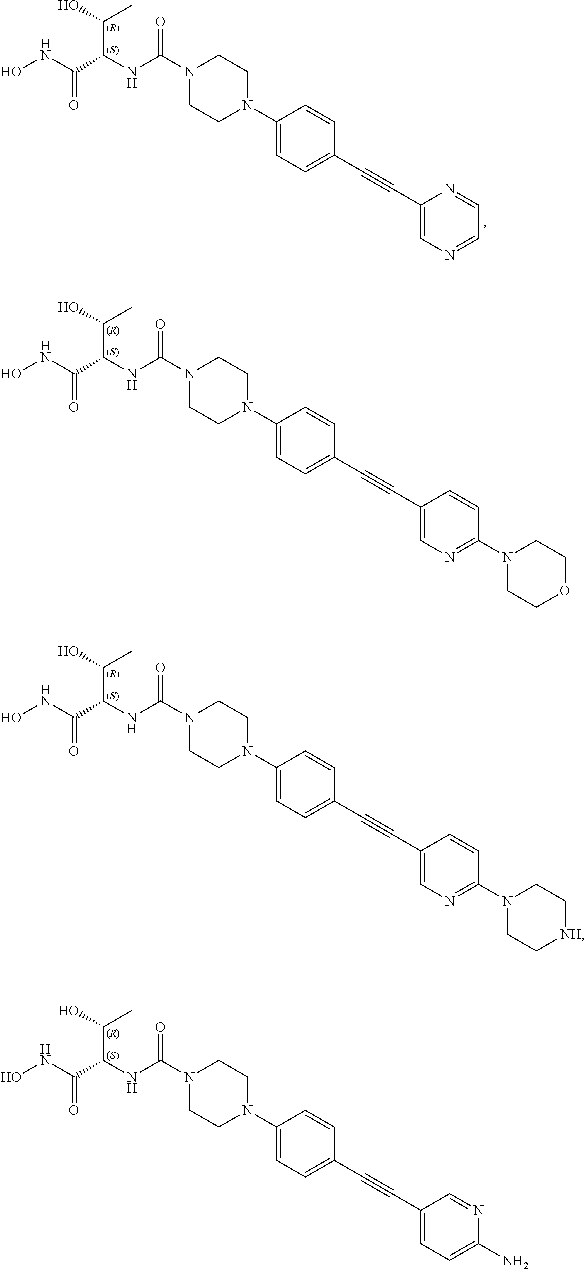 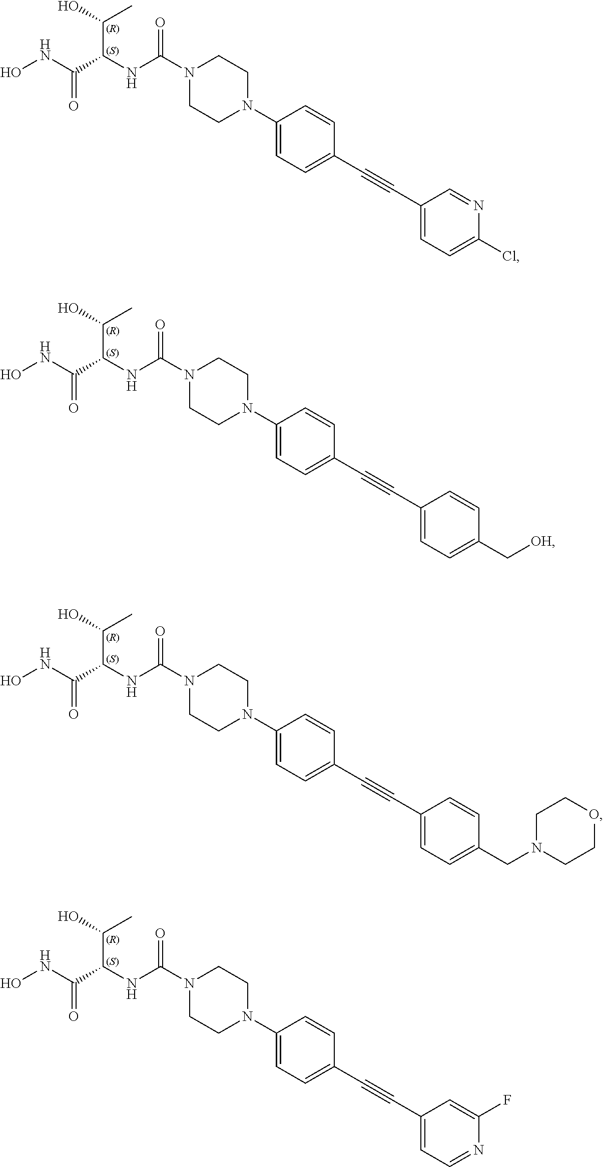 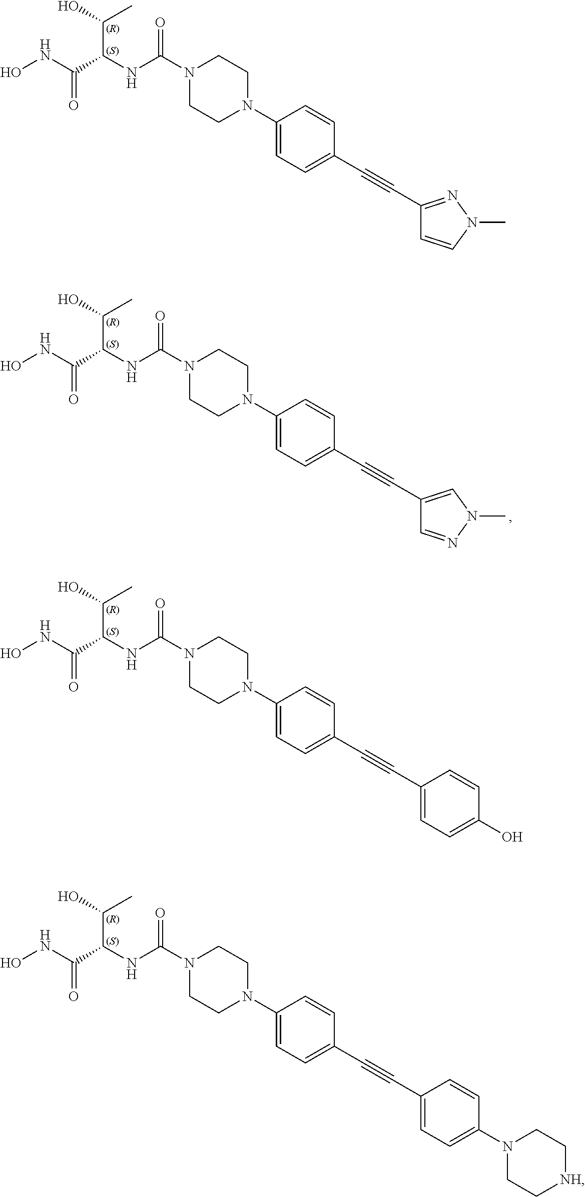 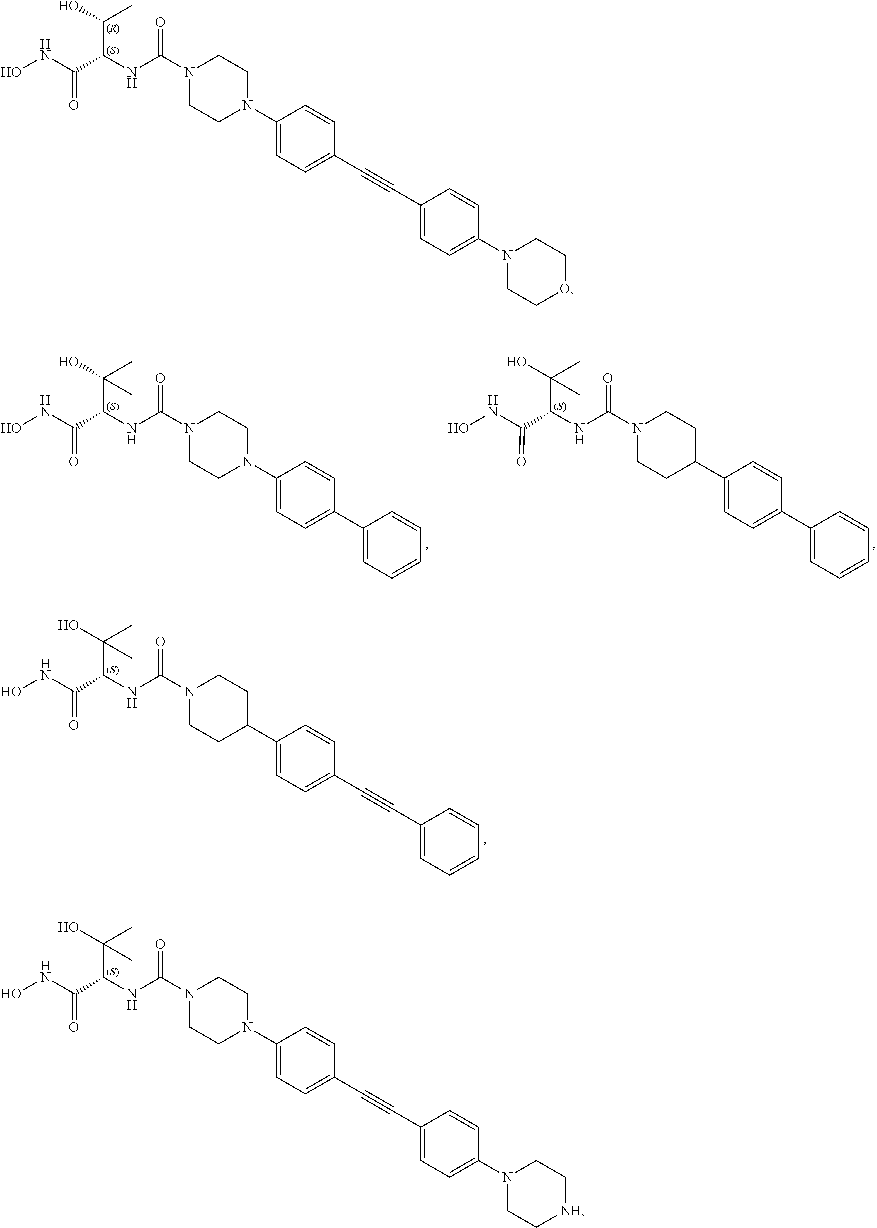 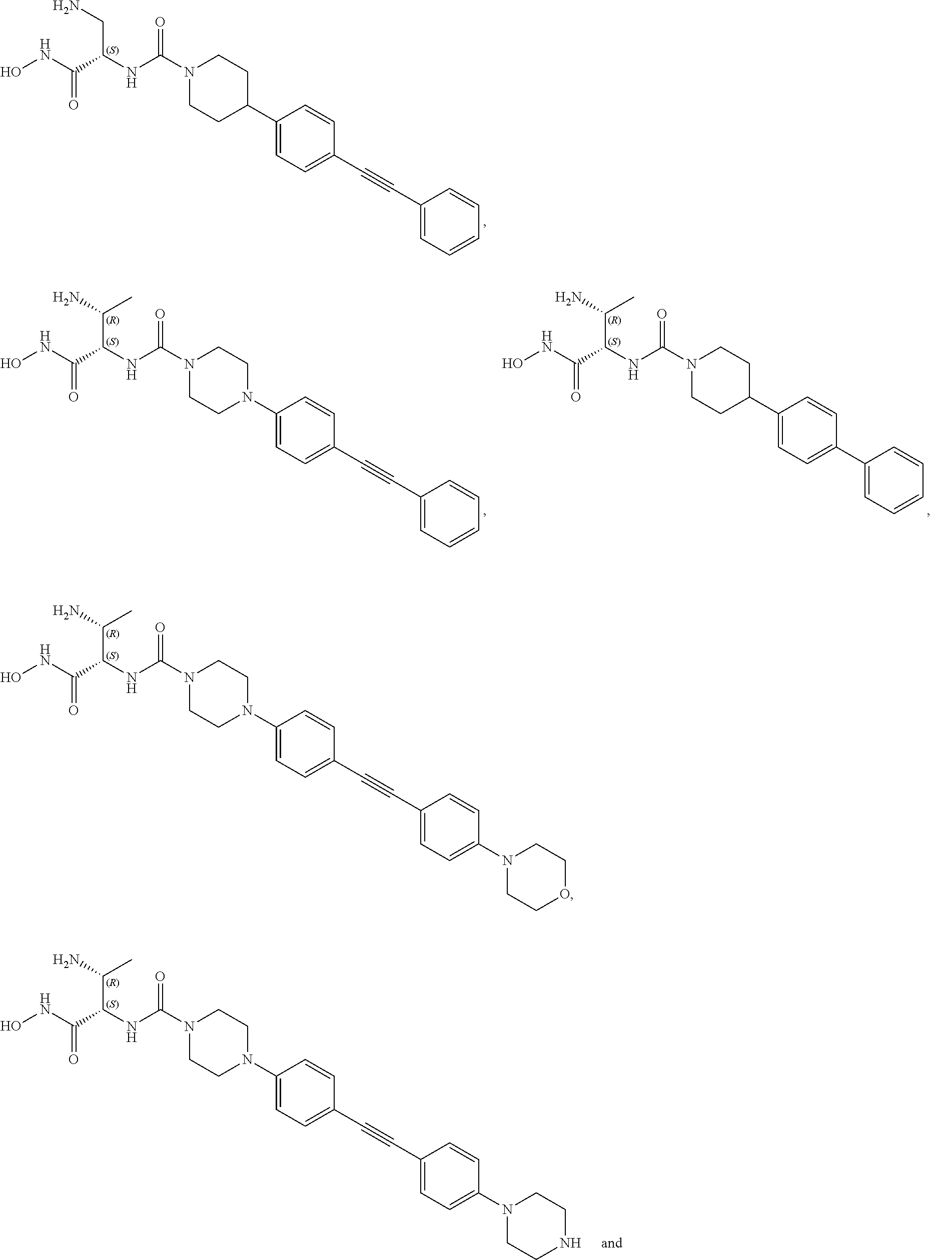 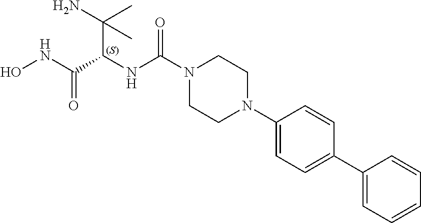
or a pharmaceutically acceptable salt, solvate or ester thereof.

17. (canceled)

18. A pharmaceutical composition comprising at least one compound of claim 1, or a pharmaceutically acceptable salt, solvate or ester thereof, in combination with at least one pharmaceutically acceptable carrier.

19. The pharmaceutical composition of claim 18, further comprising at least one additional agent, drug, medicament, antibody and/or inhibitor for treating a UDP-3-O-(R-3-hydroxymyristoyl)-N-acetylglucosamine deacetylase (LpxC) receptor mediated disease.

20. A method of treating a disorder associated with UDP-3-O-(R-3-hydroxymyristoyl)-N-acetylglucosamine deacetylase (LpxC), said method comprising administering to a patient in need of such treatment a pharmaceutical composition of claim 18.

21. The method of claim 20, wherein said disorder is a microbial infection.

22-29. (canceled)
